(19) United States
(12) Patent Application Publication
(10) Pub. No.: US 2026/0345831 A1
(43) Pub. Date: Nov. 5, 2026

(54) RNA-GUIDED CONTROL OF PROTEIN SECRETION AND MEMBRANE PROTEIN EXPRESSION

(71) Applicant: THE UNIVERSITY OF CHICAGO, Chicago, IL (US)

(72) Inventors: Chuan HE, Chicago, IL (US); Tao PAN, Chicago, IL (US); Bei LIU, Chicago, IL (US)

(73) Assignee: THE UNIVERSITY OF CHICAGO, Chicago, IL (US)

(21) Appl. No.: 19/701,916

(22) Filed: Jun. 9, 2026

Related U.S. Application Data
(63) Continuation of application No. PCT/US2025/014154, filed on Jan. 31, 2025.
(60) Provisional application No. 63/627,314, filed on Jan. 31, 2024.

(51) Int. Cl. A61K 48/00; C12N 15/11

(52) U.S. Cl. CPC A61K 48/0066; A61K 48/0033; C12N 15/11; C12N 2310/20

(57) ABSTRACT

Aspects of the disclosure relate to nucleic acid compositions involving small nucleolar RNAs (snoRNAs), including for increasing secretion of a protein of interest. Aspects also relate to methods of using the nucleic acid compositions.

### CROSS-REFERENCE TO RELATED APPLICATIONS

This application is a continuation of International Application No. PCT/US2025/014154, filed Jan. 31, 2025, which claims priority of U.S. Provisional Application No. 63/627,314 filed Jan. 31, 2024, all of which are hereby incorporated by reference in their entirety.

### BACKGROUND OF THE DISCLOSURE

This invention was made with government support under HG012780 awarded by the National Institutes of Health. The government has certain rights in the invention.

### SEQUENCE LISTING

The instant application contains a Sequence Listing which has been submitted in XML format and is hereby incorporated by reference in its entirety. Said XML copy, created on Jun. 1, 2026, is named ARCDP0832US-Sequence-Listing.xml and is 4,500 bytes in size.

### I. FIELD

This invention relates to the field of biochemistry, genetic engineering, and medicine.

### II. BACKGROUND

snoRNAs are natural guide RNAs (FIG. 1A) of −300 residues (FIG. 1B) that recognize their cellular RNA targets in ribonucleoprotein (RNP) complexes. There are >1,000 annotated snoRNA genes in the human genome¹⁻⁴. Canonical snoRNAs are classified into three types: C/D-box, H/ACA-box snoRNA, and small Cajal body-specific RNAs (scaRNAs). The C/D-box snoRNP is composed of a snoRNA and the catalytic subunit fibrillarin (FBL), the nucleolar protein 56 (NOP56), NOP58, and small nuclear ribonucleoprotein 13 (SNU13). The H/ACA-snoRNP is composed of a snoRNA and the catalytic subunit Dyskerin Pseudouridine Synthase 1 (DKC1), H/ACA ribonucleoprotein complex subunit 1 (GAR1), subunit 2 (NHP2), and NOP10 5-7 (FIG. 1A). ScaRNAs, which share structural features with C/D- or H/ACA-box snoRNAs, contain additional motifs for localization to Cajal bodies 8. Most snoRNAs are transcribed from introns of protein-coding genes and processed through distinct pathways, while others are expressed from stand-alone genes⁹⁻¹¹.

The well-characterized function of snoRNAs is to install 2′-O-methylation (Nₘ) modifications via C/D-box snoRNAs and pseudouridine (Ψ) modifications via H/ACA-box snoRNAs in ribosomal RNA (rRNA) and small nuclear RNA (snRNA)^{2,12-14} ScaRNAs primarily guide Nₘ and Ψ installations in snRNA, stabilizing their structure and modulating interactions with pre-mRNA to influence splicing¹⁵. Each snoRNA provides one or two guide sequences for rRNA modifications⁷, which have been shown to regulate ribosome biogenesis, modulate codon recognition, and influence ribosome-ligand interactions¹⁶⁻¹⁸. Emerging evidence suggests that snoRNAs also target other RNA species including transfer RNAs (tRNAs), messenger RNAs (mRNAs), and long noncoding RNAs (lncRNAs)¹⁹⁻²³. Additionally, snoRNAs have also been shown to bind directly to proteins, impacting protein activity^{24,25}.

Approximately 80% of annotated snoRNAs in the human genome lack well-defined functions. Some snoRNAs regulate gene expression by affecting mRNA stability, editing, and splicing²⁶⁻²⁸. These snoRNA activities may not always rely on mRNA modifications. Over 50 snoRNAs are dysregulated in more than 12 cancer types²⁹. Genomic deletion of the SNORD115/116 cluster is associated with the Prader-Willi symptom (PWS)^{30,31}, a neurodegenerative disease, while mutations in SNORD118 cause leukoencephalopathy³², another neurological disorder. The mechanisms by which snoRNA deletions or mutations lead to disease remain poorly understood, largely due to the lack of tools for identifying snoRNA targets and target RNA modification status across the transcriptome.

### SUMMARY

In general, the current disclosure relates to the discovery that a SNORA73A binding motif on an mRNA can increase protein secretion and/or membrane bound expression of the protein encoded by the mRNA.

Disclosed herein are recombinant nucleic acids encoding a protein of interest. The nucleic acid may further encode a small nucleolar RNA (snoRNA) binding motif. In some aspects, the recombinant nucleic acid encodes a recombinant SNORA73A binding motif. In some aspects, the SNORA73A binding motif is a sequence that is complementary to a sequence found on a SNORA73A snoRNA. In some aspects, the SNORA73A binding motif binds to a snoRNA comprising the sequence GCUGGGCCUC. In some aspects, the SNORA73A binding motif comprises GAGGCCCA. In some aspects, the SNORA73A binding motif comprises GAGGCCCAG. In some aspects, the SNORA73A binding motif comprises GAGGCCCAGC.

In some aspects, the SNORA73A binding motif comprises a SNORA73A binding motif described herein. In some aspects, the recombinant nucleic acid encodes a recombinant SNORA73A binding motif. In some aspects, the SNORA73A binding motif comprises a nucleic acid comprising GAGGCCCA. In some aspects, the SNORA73A binding motif comprises a nucleic acid comprising GAGGCCCAG. In some aspects, the SNORA73A binding motif comprises a nucleic acid comprising GAGGCCCAGC. In some aspects, the SNORA73A binding motif comprises a nucleic acid comprising a SNORA73A binding motif described herein.

The recombinant nucleic acid can encode a protein of interest, where a wildtype nucleic acid encoding the protein of interest does not contain a snoRNA binding motif. The recombinant nucleic acid can encode a protein of interest, where a wildtype nucleic acid encoding the protein of interest does not contain a SNORA73A binding motif. For example, the recombinant nucleic acid may encode a protein whose mRNA does not naturally contain said binding motif. In some aspects, the mRNA does not naturally contain the binding motif at or proximal to the 5′ and/or 3′ terminus of the mRNA. In certain aspects, the wildtype nucleic acid encoding the protein of interest does not comprise the SNORA73A binding motif.

In certain aspects, the protein of interest comprises a therapeutic protein. In certain aspects, the protein of interest is or is not a tumor suppressor, a cytokine, or a protein associated with a disease. In certain aspects, the therapeutic protein is or is not a protein that when expressed, secreted, or bound to a membrane can increase cell survival, cell efficiency, cellular processes, or can decrease one or more causes or symptoms of a disease.

In certain aspects, the SNORA73A binding motif is or is not inserted at the 3′-terminus of the recombinant nucleic acid. In certain aspects, the SNORA73A binding motif is or is not inserted proximal to the 3′-terminus of the recombinant nucleic acid. In certain aspects, the SNORA73A binding motif is or is not inserted at the 5′-terminus of the recombinant nucleic acid. In certain aspects, the SNORA73A binding motif is or is not inserted proximal to the 5′-terminus of the recombinant nucleic acid. Proximal may be within 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, or more or any range derivable therein, nucleotides from the terminus. Proximal may be within a length that allows for binding of secretion machinery. In certain aspects, the SNORA73A binding motif is or is not inserted in the 5′ UTR of an mRNA. In certain aspects, the SNORA73A binding motif is or is not inserted in the 3′ UTR of an mRNA. In certain aspects, the SNORA73A binding motif is or is not inserted in the CDS of an mRNA. In certain aspects, the binding motif is located at a position to allow binding to a snoRNA to bind to a protein secretion complex, which can include or exclude 7SL, DKC1, and/or NOP10.

Also disclosed are methods of increasing cellular protein secretion of a protein of interest, the method comprising administering a recombinant nucleic acid encoding the protein of interest, wherein the recombinant nucleic acid comprises a recombinant SNORA73A binding motif. The recombinant nucleic acid can comprise any of the recombinant nucleic acids disclosed herein. In certain aspects, the SNORA73A binding motif comprises a nucleic acid sequence comprising GAGGCCCA. In certain aspects, the SNORA73A binding motif comprises a nucleic acid sequence comprising GAGGCCCAG. In certain aspects, the SNORA73A binding motif comprises a nucleic acid sequence comprising GAGGCCCAGC.

In certain aspects, the SNORA73A binding motif is or is not inserted at the 3′-terminus of the recombinant nucleic acid. In certain aspects, the SNORA73A binding motif is or is not inserted proximal to the 3′-terminus of the recombinant nucleic acid. In certain aspects, the SNORA73A binding motif is or is not inserted at the 5′-terminus of the recombinant nucleic acid. In certain aspects, the SNORA73A binding motif is or is not inserted proximal to the 5′-terminus of the recombinant nucleic acid.

Also disclosed are methods of decreasing cellular protein secretion of a protein of interest, the method comprising removing a SNORA73A binding motif in a nucleic acid encoding the protein of interest. In certain aspects, the SNORA73A binding motif comprises a nucleic acid sequence comprising GAGGCCCA. In certain aspects, the SNORA73A binding motif comprises a nucleic acid sequence comprising GAGGCCCAG. In certain aspects, the SNORA73A binding motif comprises a nucleic acid sequence comprising GAGGCCCAGC. In certain aspects, the protein of interest comprises a therapeutic protein. In certain aspects, the protein of interest comprises a deleterious protein. In certain aspects, the method further comprises administering the nucleic acid encoding the protein of interest to a cell.

While specific binding motifs are disclosed, it is also specifically contemplated that mismatches between the binding motif on the recombinant nucleic acid (which may be a recombinant mRNA) and the snoRNA sequence that binds to mRNA (which may comprise GCUGGGCCUC) can occur and still mediate export, increased protein secretion, and/or membrane protein expression. For example, in some aspects, the recombinant nucleic acid comprises a recombinant binding motif comprising at least one mutation in a SNORA73A binding motif disclosed herein. In said example, the recombinant nucleic acid may still maintain increased protein secretion and/or membrane protein expression over a nucleic acid that lacks the recombinant binding motif. As another example, in some aspects, the recombinant nucleic acid comprises a recombinant binding motif comprising 1, 2, 3, 4, or 5 mutations in a SNORA73A binding motif disclosed herein. In said example, the recombinant nucleic acid may still maintain increased protein secretion and/or membrane protein expression over a nucleic acid that lacks the recombinant binding motif.

Also disclosed are recombinant small nucleolar RNA (snoRNA). The recombinant snoRNA can comprise a recombinant binding sequence complementary to a sequence on an mRNA encoding a protein of interest. For example, the recombinant snoRNA can be a wild-type snoRNA that has been engineered to comprise a sequence complementary to a sequence on an mRNA of interest. Such engineering, in some aspects, can increase the protein secretion and/or membrane protein expression of a protein encoded by the mRNA. The increase may be mediated through the protein secretion machinery described herein. In certain aspects, the sequence on the mRNA is or is not a sequence in the 3′ UTR of the mRNA. In certain aspects, the sequence on the mRNA is or is not a sequence in the 5′ UTR of the mRNA. In certain aspects, the sequence on the mRNA is or is not a sequence in the CDS of the mRNA. Also disclosed are methods of increasing protein secretion of a protein of interest and/or increasing membrane protein expression of a protein of interest, the method comprising contacting an mRNA encoding the protein of interest with a snoRNA comprising a recombinant binding sequence complementary to a sequence on the mRNA.

Also disclosed are methods of decreasing cellular protein secretion in a population of cells, the method comprising administering a SNORA73 inhibitor to the population of cells.

Also disclosed are methods of treating a disease in a patient, the method comprising administering to the patient any of the recombinant nucleic acids disclosed herein. In certain aspects, the disease is characterized by a deficiency in the protein of interest. In certain aspects, the disease is characterized by a secretion deficiency in the protein of interest.

Also disclosed are methods of sequencing snoRNA-RNA interactions. The sequencing can be performed using any of the methods disclosed herein.

Also disclosed are any of the following aspects:
A1. A recombinant nucleic acid encoding a protein of interest, the nucleic acid comprising a recombinant SNORA73A binding motif.
A2. The recombinant nucleic acid of aspect A1, wherein the SNORA73A binding motif comprises a nucleic acid sequence comprising GAGGCCCA.
A3. The recombinant nucleic acid of aspect A1, wherein the SNORA73A binding motif comprises nucleic acid sequence comprising GAGGCCCAG.
A4. The recombinant nucleic acid of aspect A1, wherein the SNORA73A binding motif comprises nucleic acid sequence comprising GAGGCCCAGC.
A5. The recombinant nucleic acid of any one of aspects A1 to A4, wherein a wildtype nucleic acid encoding the protein of interest does not comprise the SNORA73A binding motif.
A6. The recombinant nucleic acid of any one of aspects A1 to A5, wherein the protein of interest comprises a therapeutic protein.
A7. The recombinant nucleic acid of any one of aspects A1 to A6, wherein the SNORA73A binding motif is at the 3′ terminus of the recombinant nucleic acid.
A8. The recombinant nucleic acid of any one of aspects A1 to A6, wherein the SNORA73A binding motif is proximal to the 3′-terminus of the recombinant nucleic acid.
A9. A vector comprising the recombinant nucleic acid of any one of aspects A1 to A8.
A10. A cell comprising the recombinant nucleic acid of any one of aspects A1 to A8 or the vector of aspects A9.
A11. A composition comprising the recombinant nucleic acid of any one of claims A1 to A8, the vector of A9, or the cell of A10.
A12. A pharmaceutical composition comprising the recombinant nucleic acid of any one of claims A1 to A8, the vector of A9, or the cell of A10.
A13. The pharmaceutical composition of aspect A12, further comprising a pharmaceutical excipient.
B1. A recombinant small nucleolar RNA (snoRNA) comprising a recombinant binding sequence complementary to a sequence on an mRNA encoding a protein of interest.
B2. The recombinant snoRNA of aspect B1, wherein the sequence on the mRNA is a sequence in the 3′ UTR of the mRNA.
B3. The recombinant snoRNA of aspect B1, wherein the sequence on the mRNA is a sequence in the 5′ UTR of the mRNA.
B4. The recombinant snoRNA of aspect B1, wherein the sequence on the mRNA is a sequence in the CDS of the mRNA.
C1. A method of increasing protein secretion of a protein of interest and/or increasing membrane protein expression of a protein of interest, the method comprising contacting an mRNA encoding the protein of interest with a snoRNA comprising a recombinant binding sequence complementary to a sequence on the mRNA.
C2. The method of aspect C1, wherein the protein of interest is a tumor suppressor protein, a cytokine, or a therapeutic protein.
C3. The method of aspect C1 or C2, wherein the increasing occurs in a cell.
C4. The method of aspect C3, wherein the cell is a human cell.
D1. A method of increasing cellular protein secretion of a protein of interest and/or membrane protein expression of a protein of interest in a cell, the method comprising administering to the cell a recombinant nucleic acid encoding the protein of interest, wherein the recombinant nucleic acid comprises a recombinant SNORA73A binding motif.
D2. The method of aspect D1, wherein the SNORA73A binding motif comprises a nucleic acid sequence comprising GAGGCCCA.
D3. The method of aspect D1, wherein the SNORA73A binding motif comprises a nucleic acid sequence comprising GAGGCCCAG.
D4. The method of aspect D1, wherein the SNORA73A binding motif comprises a nucleic acid sequence comprising GAGGCCCAGC.
D5. The method of any one of aspects D1 to D4, wherein a wildtype nucleic acid encoding the protein of interest does not comprise the SNORA73A binding motif.
D6. The method of any one of aspects D1 to D5, wherein the protein of interest comprises a therapeutic protein.
D7. The method of any one of aspects D1 to D6, wherein the SNORA73A binding motif is inserted at the 3′-terminus of the recombinant nucleic acid.
D8. The method of any one of aspects D1 to D6, wherein the SNORA73A binding motif is inserted proximal to the 3′-terminus of the recombinant nucleic acid.
D9. The method of any one of aspect D1 to D8, wherein the cell is patient's cell.
D10. The method of aspect D9, wherein the administering is in vivo in the patient.
D11. The method of aspect D9, wherein the administering is ex vivo from the patient.
D12. The method of aspect D11, further comprising administering the cell to the patient.
D13. The method of cany one of aspect D1 to D8, wherein the cell is an allogeneic cell relative to a patient.
D14. The method of aspect D13, further comprising administering the cell to the patient.
D15. The method of any one of aspects D9 to D14, wherein the patient is human patient.
E1. A method of decreasing cellular protein secretion of a protein of interest and/or membrane protein expression of a protein of interest, the method comprising removing a SNORA73A binding motif in a nucleic acid encoding the protein of interest.
E2. The method of aspect E1, wherein the SNORA73A binding motif comprises a nucleic acid sequence comprising GAGGCCCA.
E3. The method of aspect E1, wherein the SNORA73A binding motif comprises a nucleic acid sequence comprising GAGGCCCAG.
E4. The method of aspect E1, wherein the SNORA73A binding motif comprises a nucleic acid sequence comprising GAGGCCCAGC.
E5. The method of any one of aspects E1 to E4, wherein the protein of interest comprises a therapeutic protein.
E6. The method of any one of aspects E1 to E4, wherein the protein of interest comprises a deleterious protein.
E7. The method of any one of aspects E1 to E6, further comprising administering the nucleic acid encoding the protein of interest to a cell.
E8. The method of aspect E7, wherein the administering is done in vivo in a patient.
E9. The method of aspect E7, wherein the administering is done ex vivo.
E10. The method of aspect E9, further comprising administering the cell to a patient.
E11. The method of any one of aspects E8 to E10, wherein the patient is a human patient.
F1. A method of decreasing cellular protein secretion in a population of cells, the method comprising administering a SNORA73 inhibitor to the population of cells.
F2. The method of aspect F1, wherein the SNORA73 inhibitor is an antisense oligonucleotide comprising a sequence complementary to SNORA73.
F3. The method of aspect F1 or F2, wherein the population of cells is a human population of cells.
G1. A method of treating a disease in a patient, the method comprising administering to the patient any of the recombinant nucleic acids disclosed herein, any one of the vectors disclosed herein, any one of the cells disclosed herein, any one of the compositions disclosed herein, or any one of the pharmaceutical compositions disclosed herein.
G2. The method of aspect G1, wherein the disease is characterized by a deficiency in the protein of interest.
G3. The method of aspect G1, wherein the disease is characterized by a secretion deficiency in the protein of interest.
G4. The method of any one of aspects G1 to G3, wherein the patient is a human patient.
G5. The method of any one of aspects G1 to G4, wherein the nucleic acid comprises the nucleic acid of any one of aspects A1 to A8 and/or any one of the recombinant snoRNAs of aspects B1 to B4.
G6. The method of any one of aspects G1 to G4, wherein the vector comprises the vector of aspect A9.
G7. The method of any one of aspects G1 to G4, wherein the cell comprises the cell of aspect A10.
G8. The method of any one of aspects G1 to G4, wherein the composition comprises the composition of aspect A11.
G9. The method of any one of aspects G1 to G4, wherein the pharmaceutical composition comprises the pharmaceutical composition of aspect A12 or A13.
H1. A method of treating a disease in a patient, the method comprising administering a cell comprising any of the recombinant nucleic acids disclosed herein.

Throughout this application, the term “about” is used according to its plain and ordinary meaning in the area of cell and molecular biology to indicate that a value includes the standard deviation of error for the device or method being employed to determine the value.

The use of the word “a” or “an” when used in conjunction with the term “comprising” may mean “one,” but it is also consistent with the meaning of “one or more,” “at least one,” and “one or more than one.” Any term used in singular form also comprise plural form and vice versa.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” “(x and z) or y,” or “x or y or z.” It is specifically contemplated that x, y, or z may be specifically excluded from an aspect or aspect.

The words “comprising” (and any form of comprising, such as “comprise” and “comprises”), “having” (and any form of having, such as “have” and “has”), “including” (and any form of including, such as “includes” and “include”), “characterized by” (and any form of including, such as “characterized as”), or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments and aspects described in the context of the term “comprising” may also be implemented in the context of the term “consisting of” or “consisting essentially of.”

It is contemplated that any aspect discussed in this specification can be implemented with respect to any method or composition of the invention, and vice versa. Furthermore, compositions of the invention can be used to achieve methods of the invention.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Use of the one or more sequences or compositions may be employed based on any of the methods described herein. Other aspects and embodiments are discussed throughout this application. Any embodiment or aspect discussed with respect to one aspect of the disclosure applies to other aspects of the disclosure as well and vice versa.

It is specifically contemplated that any limitation discussed with respect to one embodiment or aspect of the invention may apply to any other embodiment or aspect of the invention. Furthermore, any composition of the invention may be used in any method of the invention, and any method of the invention may be used to produce or to utilize any composition of the invention. Aspects of an embodiment set forth in the Examples are also aspects that may be implemented in the context of aspects discussed elsewhere in a different Example or elsewhere in the application, such as in the Summary, Brief Description of the Drawings, Detailed Description, and/or Claims.

Other objects, features and advantages of the present invention will become apparent from the following detailed description. It should be understood, however, that the detailed description and the specific examples, while indicating specific aspects of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-1F: The cDNA enrichment strategy in snoKARR-seq to capture snoRNA targets transcriptome-wide. (A) Schematics of C/D-box and H/ACA-box snoRNPs interacting with their rRNA targets. (B) Size distribution of snoRNAs. (C) An enrichment step introduced after the RT step in snoKARR-seq. Biotinylated ASO probes (grey) are designed to pair with snoRNA cDNA sequences (orange) within cDNA chimeras. (D) Enrichment efficiency of snoRNA using 50 and 100 ASOs in HepG2 cells. See also FIG. 8D. (E) RT-qPCR results showing the enrichment efficiency of five representative snoRNAs. (F) Proportions of snoRNA chimeric reads relative to total chimeric reads across human cell lines. The PARIS2 data were derived from a prior study⁵⁴. Data are represented as mean±s.d. for n=2 (D) or n=4 (E) biological replicates.

FIGS. 2A-2G: SnoKARR-seq identifies snoRNA-rRNA interactions. (A) Representative snoRNA (SNORD49A and SNORA57) rRNA interactions (left). The coverage of snoRNA on rRNA (Nₘ and Ψ sites denoted by dashed lines) detected by RIC-seq, PARIS, standard KARR-seq, or snoKARR-seq is shown on the right. (B) Signal-to-noise ratios calculated for known snoRNA-rRNA interactions from snoKARR-seq (raw data), KARR-seq, PARIS, or RIC-seq. (C) (D) 2D heat map of the two representative snoRNA-rRNA interactions from snoKARR-seq raw data (C) and after DDI processing (D). The binding sites of rRNA on snoRNAs are highlighted by the grey boxes. (E) Primers designed to amplify chimeric sequences (top), and snoRNA-rRNA interactions validated by RT-qPCR using these primers. (F) 33% of the newly identified C/D-box snoRNA rRNA targets overlap with known Nₘ sites in rRNA. (G) Free energies of known C/D-box snoRNA-rRNA duplexes and the corresponding newly identified snoRNA-rRNA interactions that overlap with known Nₘ sites, under relaxed or enforced rRNA rules. Data in (E) are represented as mean±s.d. for n=3 biological replicates. The p-values in (B and G) were determined by a paired two-tailed t-test.

FIGS. 3A-3L: SnoKARR-seq identifies snoRNA-mRNA interactions. (A) Different types of RNA targets identified for tested snoRNAs across different cell lines. The numbers in the parentheses represent the number of DG groups for each RNA type, with percentages calculated based on the number of DG groups. (B) GO analysis of conserved U3 mRNA targets across five cell lines. (C) Examples of conserved U3 mRNA targets. Results from two biological replicates are shown. (D) Predicted secondary structure of SNORD3A using R2DT⁹³, highlighting the pre-rRNA binding sites (blue) and two newly identified mRNA binding motifs (MBM). (E) Two consensus motifs of conserved U3 mRNA targets and their corresponding MBM-mRNA duplex structures. (F) Scatter plot of mRNA Nₘ fractions in control (siControl) and FBL KD (siFBL) HepG2 cells¹⁹, highlighting the overlap (pink) between C/D-box snoRNA mRNA targets and FBL-dependent mRNA Nₘ sites¹⁹. (G) Examples of snoRNA binding sites that overlap with mRNA Nₘ sites (denoted by dashed lines) in KARR-seq and snoKARR-seq. (H) Distribution of snoRNAs with targets overlapping mRNA Nₘ sites in HepG2 cells. (I) mRNA Nₘ levels quantified by LC-MS/MS in control (Ctrl) and U3 or U8 KD (ASO U3 and ASO U8) cells. (J) Free energies of known snoRNA-rRNA duplexes and newly identified snoRNA-mRNA interactions overlapping with mRNA Nₘ sites, under relaxed (with DDI processing) or enforced rRNA rules. (K) Examples of predicted snoRNA-mRNA interaction structures when applying (Nₘ rRNA rules) or disregarding (relaxed rules with DDI processing) the rRNA binding rules. Nₘ sites are denoted by red dots. (L) No overlap between mRNA targets of enriched H/ACA-box snoRNAs and mRNA Ψ I sites in HepG2 cells⁶⁷. Data in (I) are represented as mean±s.d. for n=3 biological replicates. **: p<0.01, determined by a paired two-tailed t-test.

FIGS. 4A-4G: SNORA73 targets mRNAs that encode secretory and membrane proteins. (A) The predicted structure of SNORA73A using R2DT 93, highlighting the canonical Ψ pocket (blue), mRNA binding motif (MBM, red), and the 7SL binding motif (7BM, yellow). (B) The consensus sequence motif of SNORA73 mRNA targets. (C) The SNORA73A MBM-mRNA duplex structure. See also FIG. 11C. (D) GO analysis of SNORA73 mRNA targets in HepG2 cells. (E) Metagene plot of the transcriptome-wide distribution of SNORA73 binding sites on mRNA. (F) Fractions of SNORA73 mRNA targets encoding either secretory or membrane proteins in HepG2 cells. (G) Free energies of RNA duplexes between SNORA73 MBM and the secretory protein mRNA targets. Secretory protein mRNAs selected for analysis are highlighted in red.

FIGS. 5A-5H: SNORA73 facilitates protein secretion. (A) Representative SNORA73 binding sites on mRNAs encoding secretory proteins (left) and the corresponding SNORA73-mRNA duplex structures (right). The genomic location of the mRNA from the corresponding DG group is shown. The MBM is highlighted by red boxes in the duplex structure. A representative profile from one replicate is shown. (B) Extracellular protein levels in control (LNA Ctrl) and SNORA73 KD (two independent LNA-1 and LNA-2) HepG2 cells measured by ELISA from cell culture media. (C) Extracellular and intracellular levels of secretory proteins in control (LNA Ctrl) and SNORA73 KD (LNA-1 and LNA-2) HepG2 cells. (D) Secretion levels of CLU and ALB in cell culture media measured by ELISA in control (LNA Ctrl), SNORA73 KD (LNA-1 and LNA-2) cells with overexpression of a control vector (OE Ctrl), SNORA73A (OE 73A) or SNORA73B (OE 73B). (E) The SRP-guided, co-translational translocation pathway. (F) SNORA73-7SL interactions captured by snoKARR-seq. The SNORA73-7SL duplex regions are highlighted by grey boxes. A representative profile from one replicate is shown. (G) The SNORA73A-7SL duplex structure. See also FIG. 11C. (H) Secretion levels of CLU and APOH were measured by ELISA in either the control (LNA Ctrl) or SNORA73 KD (LNA-1) cells, with overexpression of various constructs. These constructs include a control vector (OE Ctrl), SNORA73 7SL mutant (7SL-MUT) constructs, specifically SNORA73^{7SL-MUT} (OE 73A^{7SL-MUT}), SNORA73B^{7SL-MUT} (OE 73B^{7SL-MUT}) as well as the wild-type (WT) SNORA73 constructs SNORA73A^{WT} (OE 73A^{WT}) and SNORA73B^{WT} (OE 73B^{WT}). See also FIG. 12K, N. Data in (B, D, and H) are represented as mean±s.d. for n=3 biological replicates. *: p<0.05, **: p<0.01, determined by a paired two-tailed t-test.

FIGS. 6A-6E: The mRNA-SNORA73-7SL triad promotes protein secretion. (A) RT-qPCR quantification of snoRNAs and 7SL RNA in cytosolic ribosomes. (B) Protein levels of ribosomal protein RPL17, cytoplasmic protein β-tubulin, nuclear protein FBL, and DKC1 in different cellular fractions. (C) RIP-qPCR experiments showing the transcript levels in control (LNA Ctrl) and SNORA73 KD HepG2 cells (LNA 73-1 and LNA 73-2) using antibodies against either SRP72 or SRP14. See also FIG. 13B. (D) A reporter assay measuring the mCherry (depicted in red) fluorescence in cell culture media during the co-expression of wild-type (SNORA73^{WT}) or mutant (SNORA73^{MUT}) SNORA73A/B MBM (red) and the corresponding CLU-mCherry constructs (CLU^{WT} and CLU^{MUT}, grey). Duplex structures formed between the MBM of SNORA73^{WT}ISNORA73^{MUT} and the corresponding SNORA73 binding site in CLU^{WT}/CLU^{MUT} are illustrated below the plot, with mismatched base pairs marked by a red “X”. Mutated bases are underscored. The schematics illustrating the mRNA-SNORA 73-7SL ternary interactions are shown at the bottom. (E) A reporter assay measuring the GFP fluorescence in cell culture media during co-expression of SNORA73^{WT} or SNORA73^{MUT} with secretory GFP constructs (seGFP^{sM} and eGFP^{sM}). These constructs consist of the signal peptide sequence (“SP” in grey), the eGFP sequence (green), and the SNORA73 binding sequence (“sM” in cyan). The construct GFP^{sM} lacks the signal peptide. Data are represented as mean±s.d. for n=3 (A, C, E) or 4 (D) biological replicates. *: p<0.05, **: p<0.01, determined by a paired two-tailed t-test.

FIGS. 7A-7B: The proposed snoRNA-mediated protein translocation model. (A) The mRNA-snoRNA-7SL triad promotes the recruitment of SRP to the translation machinery, enhancing ER translocation and protein secretion. Recruitment of SRP occurs after the synthesis of the nascent peptide and the signal sequence (solid arrows). Alternatively, in the pathway depicted by dashed lines, SRP is recruited to the mRNA concurrently with the ribosome, before nascent peptide synthesis. (B) RT-qPCR quantification of SNORA73 targets (CLU, LGALS3BP, SERPINA1, and ALB) and a non-target (APOH) in the cytosolic ribosome fraction versus cytosolic input in control (LNA Ctrl) and SNORA73 KD (LNA-1 and LNA-2) HepG2 cells. Data are represented as mean±s.d. for n=3 biological replicates.

FIGS. 8A-8H. ASO enrichment of snoRNAs in different cell lines, related to FIG. 1. (A) Distribution of intra-molecular chimeric reads for different RNA types from HepG2 KARR-seq data. (B) Distribution of the average gap length between two predicted unpaired guanosines in chimeric reads (left). Length distribution of chimeric reads from KARR-seq data (right). (C) Abundance of different snoRNAs in 4 cell lines (HepG2, HEK293T, PC3, A549, and MDA-MB-231) from small RNA-seq data. (D) Top 50 abundant snoRNAs enriched using 50 ASOs in a single experiment across different cell lines. Data are represented as mean±s.d. for n=2 biological replicates. (E) Comparison of snoRNA abundance and enrichment efficiency (left). Correlation between snoRNA cDNA-ASO interaction free energy and snoRNA enrichment efficiency; r is Pearson's correlation (right). SNORD3A, SNORD3D, and SNORD3B-2 are three variants of U3. Although their sequences are nearly identical, and the ASO probes can enrich any of the three, the higher abundance of SNORD3A likely leads to more frequent ASO binding, explaining its higher enrichment efficiency. SNORD3D and SNORD3B-2 were excluded in the correlation plot shown on the right. (F) Distribution of RNA types of enriched chimeric reads from snoKARR-seq, derived from HepG2 cells using 50 ASOs. (G) Read count correlation of genomic coverage in two replicates of snoKARR-seq across five different cell lines. (H) Unique chimeric read count correlation in two replicates of snoKARR-seq across five different cell lines. r represents Pearson's correlation.

FIGS. 9A-9D: SnoRNA-rRNA interactions detected by snoKARR-seq, related to FIG. 2. (A) Interaction profiles for known snoRNA-rRNA interactions from KARR-seq, snoKARR-seq raw data, and snoKARR-seq with de novo structure identification and proximity interaction removal (snoKARR-seq DDI+PIR). Zoomed-in regions of snoRNA binding sites on rRNA from two replicates (rep1 and rep2) are shown. 41 snoRNAs enriched have known snoRNA-rRNA interactions that lead to either Nₘ or Ψ (denoted as dashed lines). (B) Representative interaction profiles for snoRNAs with known rRNA targets from KARR-seq, snoKARR-seq raw data, and snoKARR-seq with de novo structure identification and proximity interaction removal (snoKARR-seq DDI+PIR). Full rRNA (185, 5.85, and 28S) regions from two replicates are shown. (C) Representative interaction profiles for snoRNAs without known rRNA targets from KARR-seq, snoKARR-seq raw data, and snoKARR-seq with de novo structure identification (snoKARR-seq DDI). Full rRNA (185, 5.85, and 28S) regions from two replicates are shown. Because SNORD3A, SNORD3C, SNORD3D, and SNORD3B-2 are different variants of U3, the profile for SNORD3A is shown as an example. (D) Representative snoRNA-rRNA interaction profiles from snoKARR-seq raw data, without and with rRNA depletion, and post-reverse-crosslinking ligation snoKARR-seq data (reverse-crosslink-ligation). Some raw snoKARR-seq profiles from (B) are reused in this panel.

FIGS. 10A-10L: SnoRNA targets identified in different human cell lines and mouse brain tissue, related to FIG. 3. (A) Distribution of snoRNAs with identified targets (DG groups) in different cell lines. (B) Distribution of mRNA (from DG groups) regions targeted by tested snoRNAs in different cell lines. The p-values were calculated from Fisher's exact test to assess the enrichment of target mRNAs in the 5′ UTR, comparing snoKARR-seq mRNA target sites with randomly shuffled mRNA regions to determine snoRNA target enrichment in the 5′ UTR. (C) snoRNA-target interactions (left) and target type (right) conserved in humans, chimpanzees, and mice. (D) Shared snoRNA-mRNA and snoRNA-lncRNA interactions identified in different human cell lines. (E) Most shared mRNA targets are associated with U3 and SNORA73. (F) Abundance comparison of shared snoRNA targets versus non-shared targets. (G) Counts of DG clusters of snord115/116 targets identified in mouse brain tissue. (H) The number of different types of snord115/116 targets identified in mouse brain samples. (I) Examples of snord115/116 targets identified in mouse brain samples. (J) KD efficiency of ASO targeting U3 and U8. Data are represented as mean±s.d. for n=3 biological replicates. **: p<0.01 determined by a paired two-tailed t-test. (K) Representative snoRNA-mRNA interactions that overlap with mRNA Nₘ sites. These interactions may follow the rules of snoRNA-rRNA interactions that lead to rRNA Nₘ. (L) The distribution of nucleotide distances between the snoRNA-mRNA duplex region and the snoRNA C/D-box sequence positions. The distances within snoRNA-mRNA duplexes leading to Nₘ (Nₘ sites) are compared with those not resulting in Nₘ (non-Nₘ sites). Thep-value was determined by a paired two-tailed t-test.

FIGS. 11A-11E: SNORA73 targets mRNA through a non-canonical motif, related to FIG. 4. (A) Abundance of C/D-box and H/ACA-box snoRNAs in different cell lines. (B) Genomic location of SNORA73A and SNORA73B. Exons are denoted as grey boxes with blue edges, while intronic regions are represented by black lines. (C) Predicted structure of SNORA73B using R2DT 93, highlighting the canonical Ψ pocket (blue), mRNA binding motif (MBM, red), and the 7SL binding motif (7BM, yellow). (D) Conservation of the SNORA73 MBM in primates and mammals. (E) Free energies of RNA duplexes formed between SNORA73 MBM and the mRNA targets encoding membrane proteins.

FIGS. 12A-12N: SNORA73 targets mRNAs encoding secretory and membrane proteins, related to FIGS. 5, 6. (A) The design of the two LNAs targeting different regions in SNORA73 (left) and their KD efficiency (right) in HepG2 cells. (B) The design of 2′-MOE-based steric blocks of SNORA73 MBM, 7BM, and snoRNA binding sites on mRNAs. (C) ELISA and (D) Western blot showing secreted protein levels in the cell culture media. (E) Levels of SNORA73-targeting mRNAs and a non-target in control (LNA Ctrl) and SNORA73 KD (LNA-1 and LNA-2) HepG2 cells. (F) The protein levels of β-tubulin (cytosol marker), Sec61 β (ER marker), SNORA73 targets (CLU, LGALS3BP, SERPINA1, ALB) and non-target (APOH) in the cytosolic and ER fractions in control (LNA Ctrl), SNORA73 KD (LNA-1 and LNA-2) HepG2 cells in the absence and presence of MG132 treatment. Note that MG132 treatment has been reported to inhibit the translation of SERPINA1⁹⁴, which led to weaker SERPINA1 bands in the MG132-treated samples shown here. (G) SnoKARR-seq profiles of SNORA73-CLDN23, SNORA73-ARFGAP3, and the duplex structure models. The MBM of SNORA73 is highlighted in red boxes. A representative profile from one replicate is shown. (H) The protein levels of β-tubulin (cytosol marker), Sec61 β (ER marker), CLDN23, and ARFGAP3 in the cytosolic and ER fractions in control (LNA Ctrl) and SNORA73 KD (LNA-1 and LNA-2) HepG2 cells in the absence and presence of MG132 treatment. (I) The secondary structure of 7SL RNA, highlighting the region where SNORA73 binds. (J) SnoKARR-seq profiles of Snora73-7SL interactions identified in mESC. A representative profile from one replicate is shown. (K) The design of the 7SL mutant of SNORA73 (SNORA73A B^{7SL-MUT}) where the base pairs in the 7SL binding motif in the SL1 were swapped to maintain the secondary structure of SNORA73. (L) Impact of SNORA73 MBM mutation (MBM-MUT) and 7BM mutation (7BM-MUT) on SNORA73 overexpression levels. (M) The impact of SNORA73 MBM mutation (MBM-MUT) and 7BM mutation (7BM-MUT) on SNORA73 localization. (N) Secretion levels of CLU and APOH were measured by ELISA in either the control (LNA Ctrl) or SNORA73 KD (LNA-2) cells, with overexpression of various constructs. Data are represented as mean±s.d. for n=3 (A, C, M, N) or n=2 (E, L) biological replicates. * p<0.05, **: p<0.01, determined by a paired two-tailed t-test.

FIGS. 13A-13M: SNORA73-7SL interactions facilitate the recruitment of SRP to SNORA73 mRNA targets, related to FIG. 6. (A) Distribution of SNORA73, 7SL, CLU, and DKC1 in different stages of ribosomes. (B) SRP14 and SRP72 proteins in RIP-qPCR experiments when using IgG, SRP72, and SRP14 antibodies in control (LNA Ctrl) and SNORA73 KD (LNA-1 and LNA-2) cells. (C) KD efficiency of H/ACA-box snoRNA binding proteins. (D) Intracellular and extracellular secretory protein levels in control and H/ACA-box snoRNA binding protein KD cells. (E) Secreted protein levels from western blot results, normalized against APOH. (F) SNORA73A/B levels in control and snoRNA binding protein KD cells. (G) SNORA73A/B localization in control and snoRNA binding protein KD cells. (H) Cytoplasm and nucleus localization of canonical H/ACA-box snoRNA binding proteins. (I) Levels of CLU-mCherry mRNA bound by SRP when different combinations of wild-type (WT) and mutant (MUT) SNORA73 (MBM and 7BM mutants, see FIG. 6D, 12K) and CLU-mCherry (synonymous mutations on the SNORA73 binding site, see FIG. 6D) constructs were transfected into HEK293T cells. SRP14 antibody and control IgG were used to perform the immunoprecipitation experiments. The CLU-mCherry levels were quantified using primers targeting the mCherry sequences. The CLU-mCherry levels were normalized against 7SL levels to account for variations in IP efficiency. The 7SL-SNORA73 and SNORA73-CLUbase-pairing are either “matched” or “unmatched” as indicated by the cartoon shown below the plot. (J) Double pulldown experiments (SRP14 and CLU-mCherry mRNA IP) where HEK293T cells were transfected with either WT or MUT CLU-mCherry (top). SNORA73A/B levels were measured post-double pulldown (bottom). (K) Denatured RNA gel showing unlabeled SNORA73B and SNORA15A synthesized via in vitro transcription. U3 and SNORA73A were used as RNA size references. (L) Native RNA gel showing the complex formation of SNORA73B-mRNA, SNORA73B-7SL, and mRNA-SNORA73B-7SL complexes. Fluorescein-labeled short mRNA sequence (FAM-CACGAGGCCCAGC) and a small fragment of the 7SL sequence (FAM-AUGGUGACCUCCCGGG) that base pairs with SNORA73 were incubated and annealed with either SNORA73B or SNORA15A. The upshifted bands (denoted by black dashed boxes and numbers in red) correspond to the formation of complexes (models shown on the left). Red asterisks denote possible alternative conformations of the complexes. (M) A reporter assay measuring GFP fluorescence intensity in cell culture media during co-expression of SNORA73^{WT} or SNORA73^{MUT} with secretory GFP constructs (seGFP^{sM} and eGFP^{sM}) in SNORA73 KD HepG2 cells. These constructs consist of the signal peptide sequence (“SP”, grey), the eGFP sequence (green), and the SNORA73 binding sequence (“sM” in cyan). The construct GFP^{sM} lacks the signal peptide. Expression levels of WT or MUT SNORA73 are shown below the secreted GFP level plot. Data are represented as mean±s.d. for n=3 (E, F, G, J, M) or n=4 (I) biological replicates. *: p<0.05, **: p<0.01, determined by a paired two-tailed t-test.

FIGS. 14A-14K TAG-snoRNAs mediate protein translocation, related to FIG. 7 and STAR Methods. H/ACA-box snoRNAs possessing unstructured long-hinge sequences. (B) Localizations of snoRNAs in the cytoplasm and nucleus in HepG2 and HEK293T cells. (C) SNORA73-7SL interactions identified in four different cell lines. A representative profile from one replicate is shown. (D) Different H/ACA-box snoRNA-7SL interactions identified in different cell lines. (E) Secondary structure of SNORA44, highlighting the potential MBM (mRNA binding motif) and 7BM (7SL binding motif) sequences. (F) SNORA44-7SL and SNORA44-FGFR4 mRNA interactions identified by snoKARR-seq in HepG2 cells with 100 ASOs. (G) LNA-mediated SNORA44 KD does not affect FGFR4 mRNA level. (H) SNORA44 KD leads to reduced FGFR4 secretion. (I) Native gel electrophoresis showing the product of N₃-kethoxal labeled ssRNA, and dsRNA with 10 or 8 bps. The ssRNA and one strand in the dsRNAs were labeled with fluorescein (indicated as pink spheres) for visualization. (J) Size distribution of duplex lengths predicted from snoKARR-seq data. (K) Number of DG clusters of snoRNA non-rRNA interactions that are categorized as either duplex or proximal interactions. Data are represented as mean±s.d. for n=3 (B) or n=2 (G) biological replicates.

FIGS. 15A-15F: C/D box snoRNA targets overlap with mRNA Nₘ sites. (A) Scatter plot of Nₘ-Mut-seq data shows reduced Nₘ fractions at Nₘ sites in FBL KD HepG2 cells. (B) Fraction of C/D box snoRNA targets that overlap with FBL-hypo Nₘ sites or sites that are FBL insensitive (non-FBL hypo). (C) Examples of snoRNA binding sites that overlap with mRNA Nₘ sites (denoted by dashed lines). (D) Decomposition of snoRNAs that bind to Nₘ sites on APOB, the 8 pie charts correspond to the 8 Nₘ sites of APOB in (C). (E) Nₘ levels quantified by mass-spec in control and U3, U8 KD cells. (F) Examples of predicted snoRNA-mRNA interaction structures when enforcing or neglecting the rRNA binding rules. Shown on the right is the box plot comparing the distances between mRNA binding sites on snoRNA and the snoRNA box region for mRNA sequences with and without Nₘ.

### DETAILED DESCRIPTION

Certain aspects relate to the discovery that snoKARR-seq identifies snoRNA targets transcriptome-wide. Certain aspects relate to the discovery that SNORA73 targets mRNAs through a non-canonical motif and these interactions do not result in Ψ. Certain aspects relate to the discovery that snoRNA acts as an RNA signal that enhances protein secretion. In some aspects, this is achieved by facilitating the translocation of proteins to the ER through the 7SL-snoRNA-mRNA ternary interactions. Certain aspects relate to the discover that engineering snoRNAs can boost biotherapeutic protein production. Certain aspects relate to the discovery that engineering snoRNA and mRNA can control the secretion of disease-related secretory proteins.

Protein secretion is fundamental in biology and human diseases. There is a growing interest in engineering the secretory pathway to boost biotherapeutic protein production. Protein secretion is primarily guided by N-terminal signal peptides that interact with the signal recognition particle (SRP) to facilitate co-translational deposition into the endoplasmic reticulum (ER) lumen. Up until now, however, it was unknown whether non-protein signals are present in human cells that also facilitate protein secretion. Aspects herein show that small nucleolar RNAs (snoRNA), a family of natural guide non-coding RNAs, facilitate the secretion of proteins by targeting their messenger RNAs (mRNAs) through base pairing. Additionally, aspects herein uncover base pairing interactions between snoRNAs and 7SL RNA, the RNA component of the SRP, which is critical in directing proteins to the ER. The mRNA-snoRNA and snoRNA-7SL RNA interactions enhance the association of the translation machinery with the SRP, thereby improving the efficiency of protein secretion. The significance of this aspects herein lies in the identification of specific and conserved binding sequences within the snoRNAs that can be manipulated. By engineering these sequences to target specific mRNAs, it is possible to increase or decrease the secretion of desired proteins. This innovative technology promises to refine the secretion process, offering scalable solutions for disease treatment and advancing research capabilities.

Compared to existing empirical strategies to enhance protein secretion in mammalian cells, which include cell, vector, and signal peptide engineering, snoRNA-guided protein secretion offers a more precise and versatile approach, for example, target specific mRNAs by simple base pairing design between the snoRNA and the mRNA. Traditional methods, while effective in industrial settings to enhance biopharmaceutical quantity and quality, fall short in native biological contexts due to their empirical nature and invasiveness. In contrast, snoRNA engineering allows for in vivo application, directly targeting tissue or cell-specific protein secretion through simple base-pairing rules. This method provides a safer alternative to permanent genomic editing and leverages the specificity of base pairing for high-precision targeting.

Small nucleolar RNAs (snoRNA) are non-coding RNAs known for guiding RNA modifications including 2′-O-methylation (Nₘ) and pseudouridine (Ψ). While snoRNAs may also interact with other RNAs such as mRNA, the full repertoire of RNAs targeted by snoRNA remains elusive due to the lack of effective technologies that identify snoRNA targets transcriptome-wide. Aspects herein develop a chemical crosslinking-based approach that comprehensively detects cellular RNA targets of snoRNAs, yielding thousands of previously unrecognized snoRNA-mRNA interactions in human cells and mouse brain tissues. Many interactions occur outside of snoRNA-guided RNA modification sites, suggesting non-canonical functions beyond RNA modification. Aspects herein show an snoRNAs, SNORA73, targets mRNAs that encode secretory proteins and membrane proteins. SNORA73 also interacts with 7SL RNA, part of the signal recognition particle (SRP) required for protein secretion. The mRNA-SNORA73-7SL RNA interactions enhance the association of the SNORA73-target mRNAs with SRP, thereby facilitating the secretion of encoded proteins.

### I. Proteins

As used herein, the term “wild-type” refers to the endogenous version of a molecule that occurs naturally in an organism. In some aspects, wild-type versions of a protein or polypeptide are employed.

Where a protein is specifically mentioned herein, it is in general a reference to a native (wild-type) or recombinant (modified) protein or, optionally, a protein (or mRNA encoding the protein) in which any signal sequence has been removed. The nucleotide as well as the protein, polypeptide, and peptide sequences for various genes have been previously disclosed, and may be found in the recognized computerized databases. Two commonly used databases are the National Center for Biotechnology Information's Genbank and GenPept databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

### II. Nucleic Acids

Nucleic acids of the present disclosure include those described herein, including those listed in Table A. In some aspects, 1, 2, 3, 4, 5, 6, 7, 8, 9, 10, or more (or any range derivable therein) nucleotides of the nucleic acids of Table A are mutated. In some aspects, the nucleic acid retains binding affinity to the snoRNA, including SNORA73A, after the mutation. In some aspects, the nucleic acid retains binding affinity to the SNORA73A binding motif after the mutation.

TABLE A

| | | SEQ ID |
|---|---|---|
| Name | Sequence | NO |
| SNORA73A binding motif | GAGGCCCA | 1 |
| SNORA73A binding motif | GAGGCCCAG | 2 |
| SNORA73A binding motif | GAGGCCCAGC | 3 |
| SNORA73A binding | UGGGCCUC | 4 |
| sequence | | |
| SNORA73A binding | CUGGGCCUC | 5 |
| sequence | | |
| SNORA73A binding | GCUGGGCCUC | 6 |
| sequence | | |
| SNORA73A ASO | TCCCAGAGTCCTGTCG | 7 |
| | ACAATTACTGGGGAGA | |
| | CAAACCATGCAGGA | |

In certain aspects, nucleic acid sequences can exist in a variety of instances such as: isolated segments and recombinant vectors of incorporated sequences or recombinant polynucleotides encoding a protein of interest, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, and complementary sequences of the foregoing described herein

The term “polynucleotide” refers to a nucleic acid molecule that either is recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar protein.

The nucleic acid segments, regardless of the length of the coding sequence itself, may be combined with other nucleic acid sequences, such as genes, mRNAs, promoters, polyadenylation signals, additional restriction enzyme sites, multiple cloning sites, other coding segments, and the like, such that their overall length may vary considerably. The nucleic acids can be any length. They can be, for example, 5, 10, 15, 20, 25, 30, 35, 40, 45, 50, 75, 100, 125, 175, 200, 250, 300, 350, 400, 450, 500, 750, 1000, 1500, 3000, 5000 or more nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

### III. Obtaining Nucleotides

### A. Synthesis

The nucleic acid molecules, including an the recombinant nucleic acids described herein, may be generated by nucleic acid synthesis. The recombinant nucleic acids may be synthesized using any method known in the art, such as phosphoramidite synthesis and/or solid-phase synthesis.

### B. Expression

The nucleic acid molecules may be generated by expression vectors. The expression vectors used herein may contain sequences for plasmid or virus maintenance and for cloning and expression of exogenous nucleotide sequences. Such sequences, collectively referred to as “flanking sequences” typically include one or more of the following operatively linked nucleotide sequences: a promoter, one or more enhancer sequences, an origin of replication, a transcriptional termination sequence, and a selectable marker element. Such sequences and methods of using the same are well known in the art.

### 1. Expression Systems

Numerous expression systems exist that comprise at least a part or all of the expression vectors discussed above. Prokaryote- and/or eukaryote-based systems can be employed for use with an aspect to produce nucleic acid sequences. Commercially and widely available systems include but are not limited to bacterial, mammalian, yeast, and insect cell systems. Those skilled in the art are able to express a vector to produce a nucleic acid sequence using an appropriate expression system.

### 2. Methods of Gene Transfer

Suitable methods for nucleic acid delivery to effect expression of compositions are anticipated to include virtually any method by which a nucleic acid (e.g., DNA, including viral and nonviral vectors) can be introduced into a cell, a tissue or an organism, as described herein or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466, 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877, 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium mediated transformation (U.S. Pat. Nos. 5,591,616, 5,563,055, each incorporated herein by reference); or by PEG mediated transformation of protoplasts (Omirulleh et al., 1993; U.S. Pat. Nos. 4,684,611, 4,952,500, each incorporated herein by reference); by desiccation/inhibition mediated DNA uptake (Potrykus et al., 1985). Other methods include viral transduction, such as gene transfer by lentiviral or retroviral transduction.

### 3. Host Cells

In another aspect, contemplated are the use of host cells into which a recombinant expression vector has been introduced. Vector DNA can be introduced into prokaryotic or eukaryotic cells via conventional transformation or transfection techniques. Some vectors may employ control sequences that allow it to be replicated and/or expressed in both prokaryotic and eukaryotic cells. One of skill in the art would understand the conditions under which to incubate host cells to maintain them and to permit replication of a vector. Also understood and known are techniques and conditions that would allow large-scale production of vectors, as well as production of the nucleic acids encoded by vectors.

For stable transfection of mammalian cells, it is known, depending upon the expression vector and transfection technique used, only a small fraction of cells may integrate the foreign DNA into their genome. In order to identify and select these integrants, a selectable marker (e.g., for resistance to antibiotics) is generally introduced into the host cells along with the gene of interest. Cells stably transfected with the introduced nucleic acid can be identified by drug selection (e.g., cells that have incorporated the selectable marker gene will survive, while the other cells die), among other methods known in the arts.

### IV. Administration of Therapeutic Compositions

Certain aspects of the disclosure relate to compositions and methods comprising therapeutic compositions, which can comprise one or more of the recombinant nucleic acids disclosed herein. The different therapies may be administered in one composition or in more than one composition, such as 2 compositions, 3 compositions, or 4 compositions. Various combinations of the agents may be employed.

### A. Pharmaceutical or Therapeutic Compositions

In certain aspects, the compositions or agents, including those for use in the methods disclosed herein, such as one or more nucleic acids, are suitably contained in a pharmaceutically acceptable carrier, which may be referred to as pharmaceutical compositions or also may be referred to as therapeutic compositions herein. The carrier can be non-toxic, biocompatible, and selected so as not to detrimentally affect the biological activity of the agent. The agents in some aspects of the disclosure may be formulated into preparations for local delivery (i.e. to a specific location of the body, such as the brain, nervous tissue, or other tissue) or systemic delivery, in solid, semi-solid, gel, liquid or gaseous forms such as tablets, capsules, powders, granules, ointments, solutions, depositories, inhalants and injections allowing for oral, parenteral or surgical administration. Certain aspects of the disclosure also contemplate local administration of the compositions by coating medical devices and the like.

Suitable carriers for parenteral delivery via injectable, infusion or irrigation and topical delivery include distilled water, physiological phosphate-buffered saline, normal or lactated Ringer's solutions, dextrose solution, Hank's solution, or propanediol. In addition, sterile, fixed oils may be employed as a solvent or suspending medium. For this purpose any biocompatible oil may be employed including synthetic mono- or diglycerides. In addition, fatty acids such as oleic acid find use in the preparation of injectables. The carrier and agent may be compounded as a liquid, suspension, polymerizable or non-polymerizable gel, paste or salve.

The carrier may also comprise a delivery vehicle to sustain (i.e., extend, delay or regulate) the delivery of the agent(s) or to enhance the delivery, uptake, stability or pharmacokinetics of the therapeutic agent(s). Such a delivery vehicle may include, by way of non-limiting examples, microparticles, microspheres, nanospheres or nanoparticles composed of proteins, liposomes, carbohydrates, synthetic organic compounds, inorganic compounds, polymeric or copolymeric hydrogels and polymeric micelles.

In certain aspects, the actual dosage amount of a composition administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

Solutions of pharmaceutical compositions can be prepared in water suitably mixed with a surfactant, such as hydroxypropylcellulose. Dispersions also can be prepared in glycerol, liquid polyethylene glycols, mixtures thereof and in oils. Under ordinary conditions of storage and use, these preparations contain a preservative to prevent the growth of microorganisms.

In certain aspects, the pharmaceutical compositions are advantageously administered in the form of injectable compositions either as liquid solutions or suspensions; solid forms suitable or solution in, or suspension in, liquid prior to injection may also be prepared. These preparations also may be emulsified. A typical composition for such purpose comprises a pharmaceutically acceptable carrier. For instance, the composition may contain 10 mg or less, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, antgifungal agents, anti-oxidants, chelating agents and inert gases. The pH and exact concentration of the various components the pharmaceutical composition are adjusted according to well-known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders.

In further aspects, the pharmaceutical compositions may include classic pharmaceutical preparations. Administration of pharmaceutical compositions according to certain aspects may be via any common route so long as the target tissue is available via that route. This may include oral, nasal, buccal, rectal, vaginal or topical. Alternatively, administration may be by orthotopic, intradermal, subcutaneous, intramuscular, intraperitoneal or intravenous injection. Such compositions would normally be administered as pharmaceutically acceptable compositions that include physiologically acceptable carriers, buffers or other excipients. For treatment of conditions of the lungs, aerosol delivery can be used. Volume of the aerosol may be between about 0.01 ml and 0.5 ml, for example.

An effective amount of the pharmaceutical composition is determined based on the intended goal. The term “unit dose” or “dosage” refers to physically discrete units suitable for use in a subject, each unit containing a predetermined-quantity of the pharmaceutical composition calculated to produce the desired responses discussed above in association with its administration, i.e., the appropriate route and treatment regimen. The quantity to be administered, both according to number of treatments and unit dose, depends on the protection or effect desired.

Precise amounts of the pharmaceutical composition also depend on the judgment of the practitioner and are peculiar to each individual. Factors affecting the dose include the physical and clinical state of the patient, the route of administration, the intended goal of treatment (e.g., alleviation of symptoms versus cure) and the potency, stability and toxicity of the particular therapeutic substance.

### B. Administration

In some aspects, the therapy is or is not administered intravenously, intramuscularly, subcutaneously, topically, orally, transdermally, intraperitoneally, intraorbitally, by implantation, by inhalation, intrathecally, intraventricularly, or intranasally. In some aspects, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some aspects, a unit dose comprises a single administrable dose.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels). It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In certain instances, it will be desirable to have multiple administrations of the composition, e.g., 2, 3, 4, 5, 6 or more administrations. The administrations can be at 1, 2, 3, 4, 5, 6, 7, 8, to 5, 6, 7, 8, 9, 10, 11, or 12 week intervals, including all ranges there between.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

The active compounds can be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions will typically be via any common route. This includes, but is not limited to oral, or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

Upon formulation, solutions will be administered in a manner compatible with the dosage formulation and in such amount as is therapeutically or prophylactically effective. The formulations are easily administered in a variety of dosage forms, such as the type of injectable solutions described above.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: SnoRNA-Facilitated Protein Secretion Revealed by Transcriptome-Wide snoRNA Target Identification

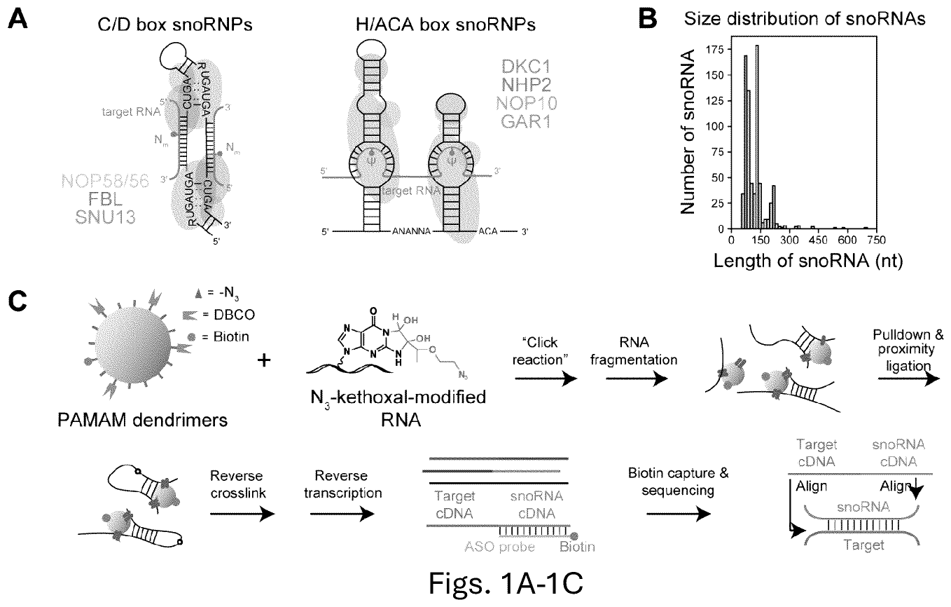

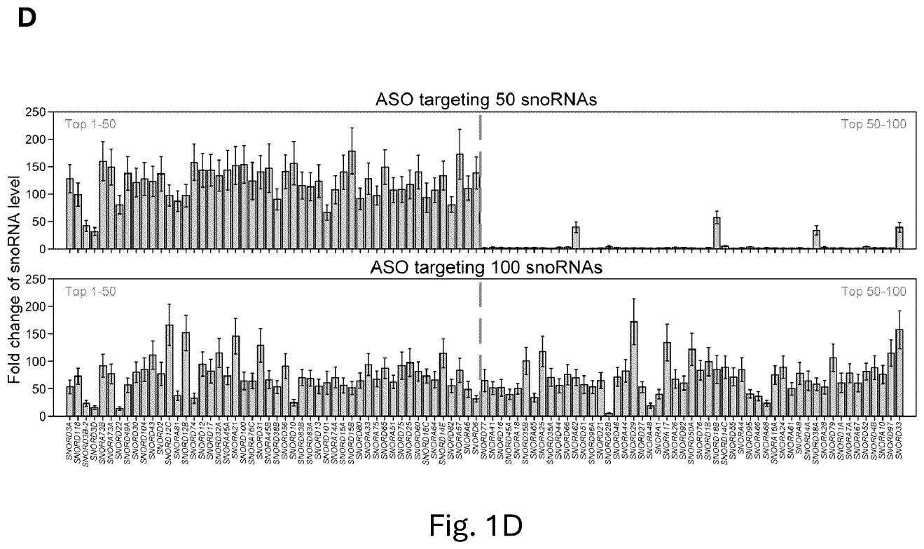

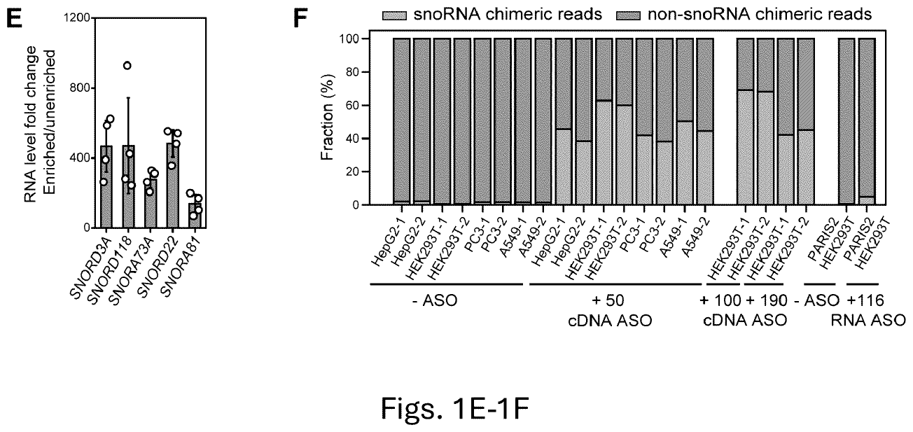

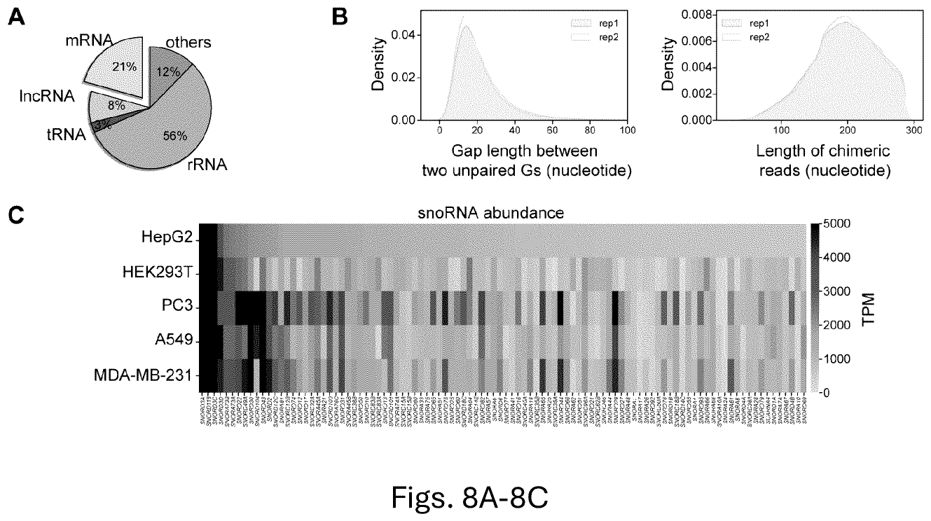

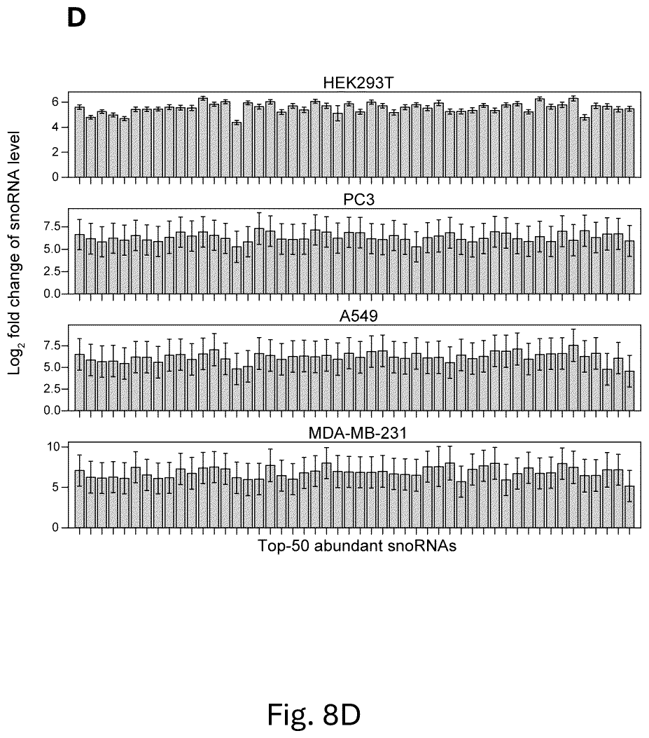

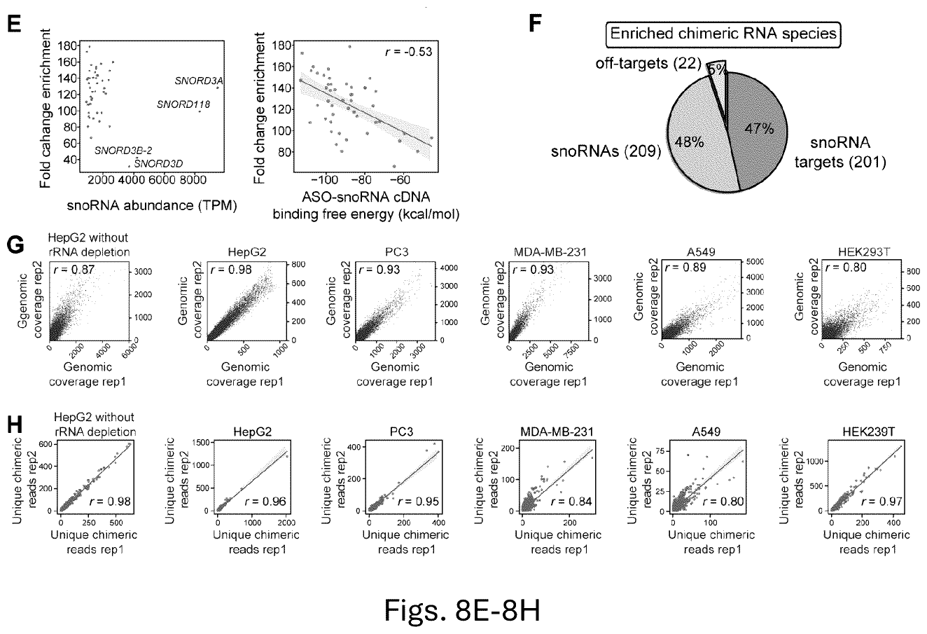

In aspects herein, the inventors present “snoKARR-seq,” a method that integrates RNA chemical labeling, crosslinking of snoRNAs with their binding RNAs, and chimeric cDNA enrichment post-reverse transcription (RT). Building on the principles of kethoxal-assisted RNA-RNA interaction sequencing (KARR-seq)³³, snoKARR-seq utilizes N₃-kethoxal to label guanosines in single-stranded RNA (ssRNA)³⁴ and dibenzocyclooctane (DBCO)-modified PAMAM dendrimers for RNA chemical crosslinking³⁵ to capture RNA-RNA interactions (FIG. 1C). N₃-kethoxal labels various RNA species (FIG. 8A) with an average distance of approximately 20 nucleotides (nt) between two labeled sites (FIG. 8B). Additionally, the inventors introduce a snoRNA-derived cDNA enrichment step in the workflow (FIG. 1C) to significantly enhance the detection specificity of chimeric reads containing snoRNA in the final sequencing library.

SnoKARR-seq detects snoRNA targets with over 100-fold higher signal-to-noise compared to conventional approaches. Using this method, the inventors identified over 1,000 previously unknown snoRNA-mRNA interactions in human cell lines and mouse brain tissue. Notably, most of these snoRNA targets do not overlap with the known mRNA modification sites^{19,36}, suggesting potential non-canonical snoRNA functions beyond RNA modification.

One unexpected interaction identified involves SNORA73 and mRNAs encoding secretory and membrane proteins. Protein secretion, a fundamental biological process, is primarily guided by N-terminal signal peptides that interact with the signal recognition particle (SRP), directing secretory proteins to the endoplasmic reticulum (ER) for co-translational translocation 37-39 While some evidence suggests signal-peptide-independent pathways⁴⁰⁻⁴⁶, the presence and roles of non-protein signals in these pathways remain unclear. SNORA73 forms stable duplexes with target mRNAs via a 10-nt motif outside the canonical guide regions; disruption of these interactions reduced protein secretion. Mechanistically, the inventors uncovered a base-pairing interaction between SNORA73 and 7SL RNA, the RNA component of SRP, revealing an mRNA-snoRNA-7SL RNA triad involved in protein translocation. Therefore, SNORA73 acts as a “molecular glue”, connecting the ribosome-nascent peptide-mRNA complex and the SRP through two distinct RNA-RNA interactions, thereby promoting protein translocation and secretion. The findings demonstrate that snoKARR-seq enables comprehensive mapping of cellular snoRNA interactome, which revealed an important role for SNORA73 in facilitating protein translocation.

### Results

The Development of snoKARR-Seq

Recently developed methods for mapping RNA-RNA interactions rely on protein-dependent (e.g. RIC-seq⁴⁷ and CLASH^{48,49}) or psoralen-mediated crosslinking (e.g. PARIS 50 and SPLASH⁵¹). While effective for studying interactions involving abundant RNAs, these methods are limited by low crosslinking efficiency and lack specific enrichment for snoRNAs, often missing snoRNA-mRNA interactions due to the relatively low abundance of snoRNAs and their target mRNAs.

Previously KARR-seq was developed, which utilizes chemical crosslinkers to effectively capture physically proximal RNAs independent of local RNA-protein interactions 33 (FIG. 1C). This approach enables de novo mapping of RNA-RNA interactions and proximity in both nuclear and cytoplasmic fractions. While KARR-seq can theoretically capture most intermolecular RNA-RNA interactions, it detects limited snoRNA-mRNA interactions due to low signal-to-noise ratios for certain low-abundance mRNAs and snoRNAs. To address this limitation, the inventors developed an improved version of KARR-seq specifically designed to enrich and detect snoRNA targets with increased sensitivity.

The main challenge lies in enriching snoRNAs within the ligated snoRNA-mRNA chimeric products in the KARR-seq library. While antisense oligonucleotides (ASOs) are traditionally used to capture specific RNAs based on sequence complementarity⁵², targeting snoRNAs is challenging due to their highly structured nature (FIG. 1A). Although snoRNAs contain ssRNA regions, these regions may be occupied by endogenous RNA targets, competing with ASOs. Additionally, designing ASOs targeting these regions necessitates prior determination of the snoRNA structure, posing a challenge in ASO design. Given these complexities, successful enrichment of snoRNAs complexed with RNA targets has been rarely achieved in the literature^{53,54}.

In the approach, the inventors enrich snoRNAs by designing ASOs to target chimeric cDNA sequences derived from snoRNAs after the RT step in the KARR-seq workflow (FIG. 1C). This design, based solely on DNA sequences, circumvents the challenges of snoRNA secondary structure. Briefly, live cells are labeled with N₃-kethoxal, which reacts with unpaired guanosines in cellular RNA close to saturation⁵⁵, followed by formaldehyde-assisted fixation. Proximal RNAs labeled by N₃-kethoxal are crosslinked by dendrimers bearing an activated alkyne group through “click” chemistry. Subsequently, proteins bound to RNAs are digested by proteinase K, and the RNAs are fragmented and enriched with streptavidin beads. Enriched RNAs undergo proximity ligation to generate chimeric RNAs, after which the kethoxal labeling is reversed³⁴. The resulting RNAs are reverse transcribed into cDNAs. Biotinylated ASOs targeting specific snoRNAs are then used to enrich the snoRNA-derived cDNAs, which are subsequently amplified for high-throughput sequencing.

Efficiency of ASO-Assisted snoRNA Target Enrichment

The inventors carried out this cDNA enrichment approach in five different cell lines (HEK293T, HepG2, PC3, A549, and MDA-MB-231) to enrich the 50 top-expressed snoRNAs identified from small RNA-seq data^{56,57} (FIG. 8C). Sequencing results confirmed successful enrichment of all 50 snoRNAs across five different cell lines (FIG. 1D, 8D), with enrichment efficiency (fold change in snoRNA abundance between enriched and unenriched libraries) ranging from 20- to 150-fold. qPCR analysis of five selected snoRNAs further validated the enrichment (FIG. 1E). Notably, the enrichment efficiency is moderately correlated with the base-pairing stability between snoRNA cDNA and ASO probe sequences (FIG. 8E).

The inventors next assessed the enrichment efficiency of snoRNAs ranked 50-100 in the small RNA-seq data. Most were not enriched when only ASOs targeting the 50 top-expressed snoRNAs were used, underscoring the high specificity of the ASO probes (FIG. 1D). A few snoRNAs outside of the top 50 (e.g. SNORA62, SNORD18B, SNORD38A, SNORD33) were also enriched, likely due to sequence similarities with the top 50 snoRNAs. Moreover, when using 100 ASOs targeting all top 100 snoRNAs, the inventors observed high enrichment efficiency for almost all snoRNAs (FIG. 1D), further confirming the effectiveness and specificity of the strategy.

The inventors next analyzed the enrichment of chimeric reads containing snoRNAs, which encode the snoRNA target information. Specifically, the inventors quantified the proportion of snoRNA-containing chimeric reads out of the total chimeric reads. While only ~1-2% of the chimeric reads contain snoRNA in the unenriched library, the inventors observed a ~20-40-fold increase in the proportion of chimeric reads containing snoRNA across different cell lines when using different amounts of ASOs to enrich snoRNA cDNA (FIG. 1F), far exceeding enrichment levels achieved with ASOs targeting RNA (FIG. 1F)⁵⁴. These results demonstrate the superior efficiency of the cDNA enrichment approach in capturing chimeric reads with target snoRNAs. Notably, only ~5% of the enriched RNAs at the whole transcriptome level are potential off-targets, likely due to partial sequence complementarity or non-specific ASO interactions (FIG. 8F).

SnoKARR-Seq Detects Known snoRNA-rRNA Interactions.

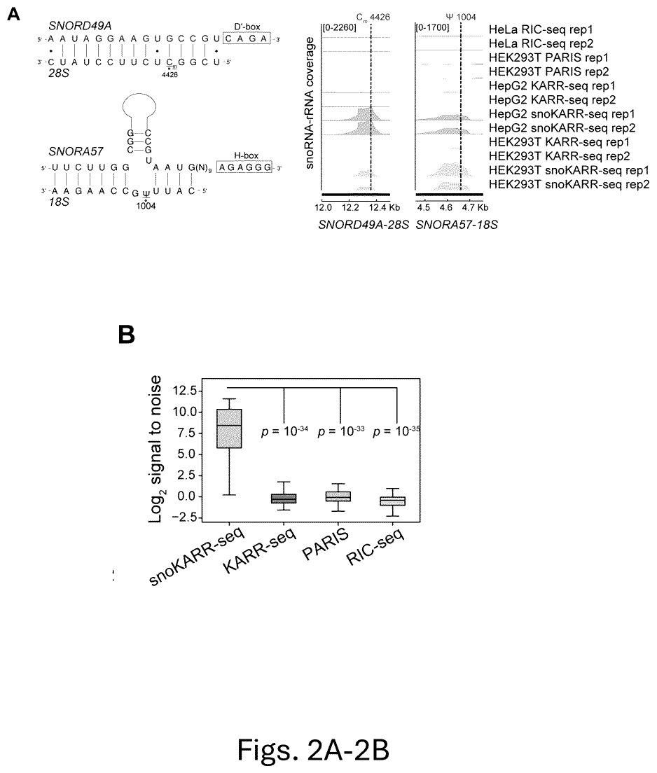

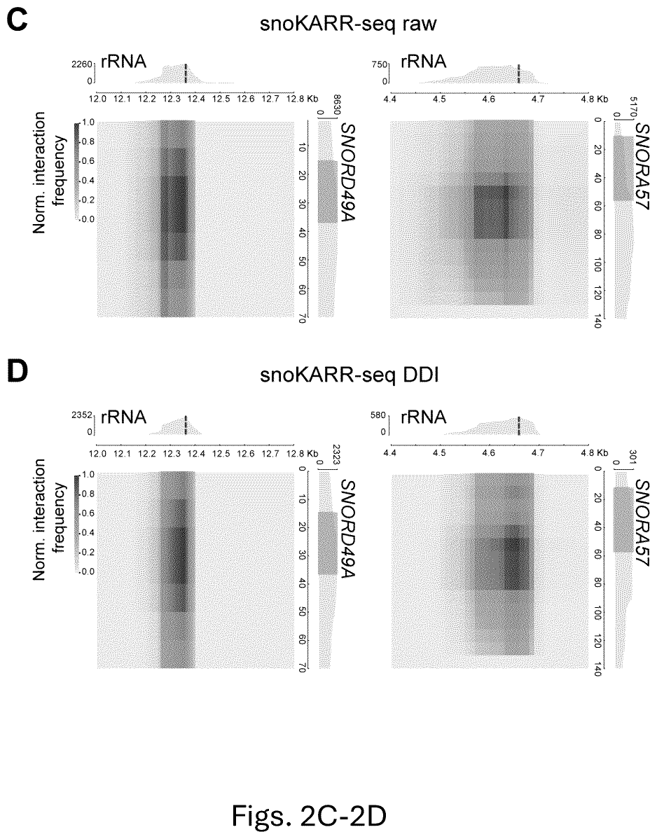

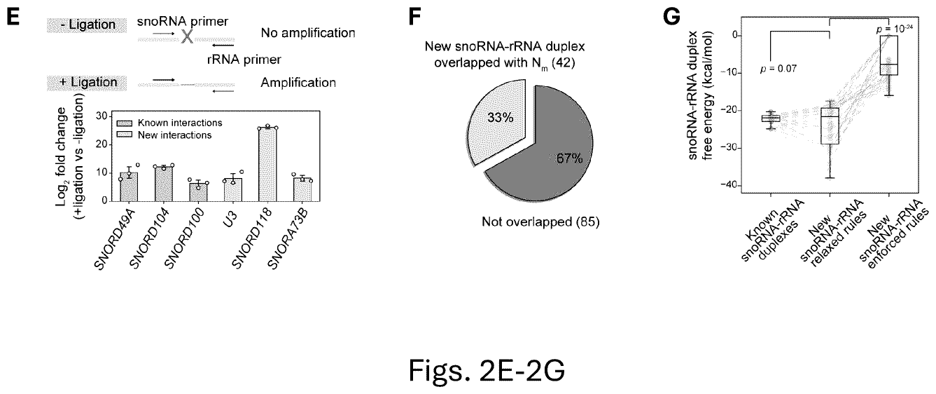

SnoRNAs are known for guiding Nₘ and Ψ installations on rRNA^{5,12-14}. The inventors used these well-characterized snoRNA-rRNA interactions as benchmarks to evaluate the performance of snoKARR-seq, which showed high reproducibility between replicates (FIG. 8G-H). The inventors focused on the top 50 expressed snoRNA (36 C/D-box and 14 H/ACA-box snoRNAs) in HepG2 cells. Of these, 41 (29 C/D-box and 12 H/ACA-box snoRNAs) have known rRNA targets, and all were successfully captured by snoKARR-seq (FIG. 2A, 9A). Notably, the modification sites align almost perfectly with the center of the peaks in the snoRNA-rRNA interaction profiles (FIG. 2A, 9A).

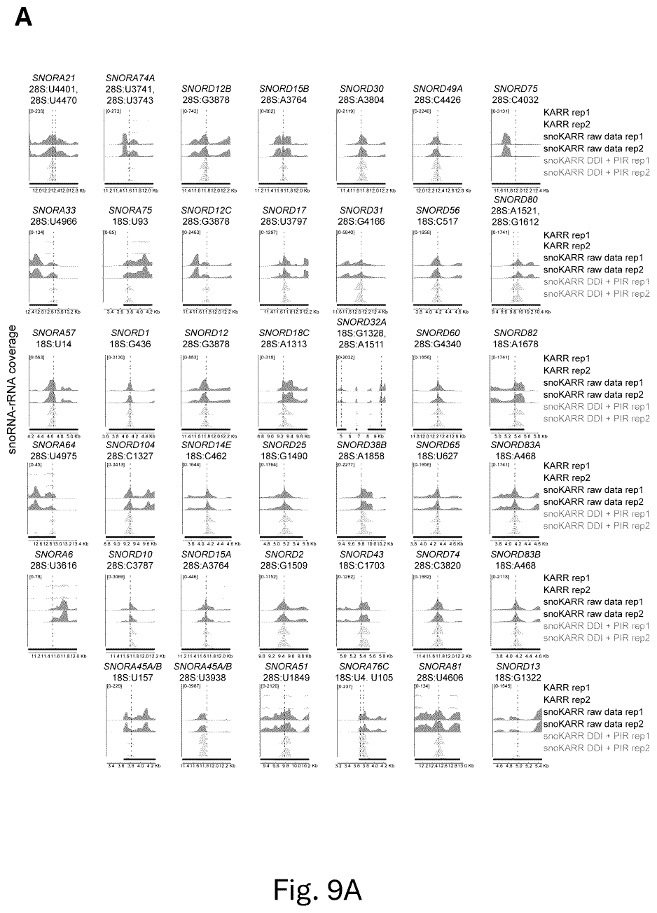

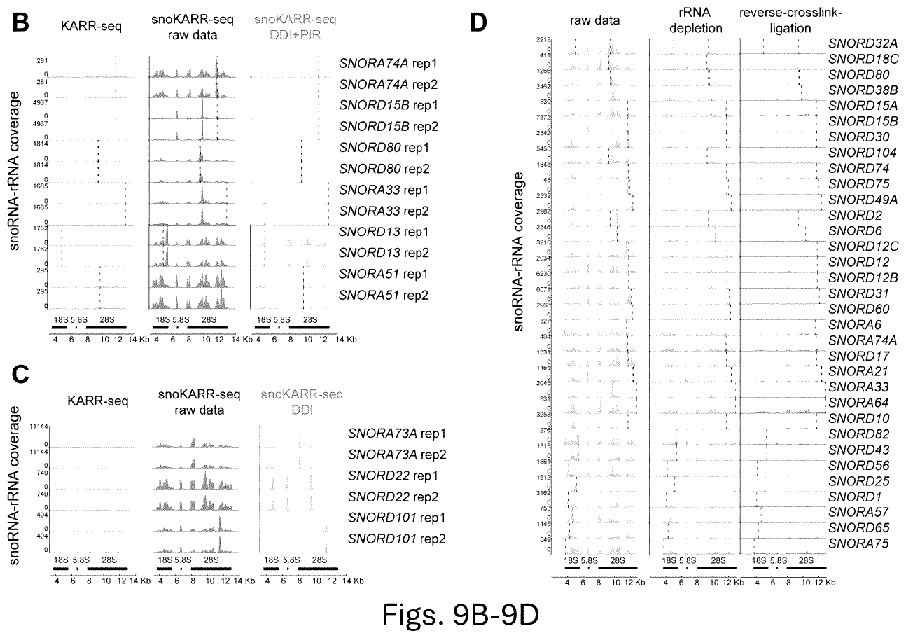

Comparative analysis with other methods^{47,50} and regular KARR-seq 33 demonstrated that snoKARR-seq achieves significantly higher signal-to-noise for known snoRNA-rRNA targets (FIG. 2A, B). The 2D interaction heatmap (FIG. 2C) displays local maxima at regions corresponding to the complementary sequences of snoRNA and rRNA modification sites, supporting direct interactions between these regions. Using double-strand group (DG) clustering 50, de novo duplex identification (DDI), and rRNA proximal interaction removal (PIR) (see STAR Methods), the inventors eliminated potential proximal interactions and recapitulated the precise snoRNA-rRNA duplex structures (FIGS. 2A, C-D, 9B-C). Some known snoRNA-rRNA interactions (e.g. SNORA33, SNORA75. FIG. 9A) exhibited relatively weak signals, likely due to modification sites residing near the rRNA ends, resulting in poor coverage from limited N₃-kethoxal labeling.

Beyond the previously characterized snoRNA-rRNA interactions responsible for Nₘ or Ψ modifications, the inventors identified numerous previously unknown snoRNA-rRNA interactions (FIG. 9B-C). To validate these interactions and rule out sequencing artifacts or spurious ligation, the inventors conducted snoKARR-seq with rRNA depletion or post-reverse-crosslinking ligation, where RNA labeling by kethoxal was reversed prior to proximity ligation. Both experiments resulted in reduced snoRNA-rRNA interaction signals (FIG. 9D). Additionally, RT-qPCR targeting snoRNA-rRNA interaction regions was performed on samples with and without proximity ligation (FIG. 2E). Successful amplification of chimeric cDNA confirmed both known and newly identified interactions (FIG. 2E). These newly identified snoRNA-rRNA interactions may not lead to rRNA modification as they do not appear to follow the established rules (the Nₘ modification site typically lies 5-nt upstream of the D box^{14,58}) (FIG. 2F-G). Instead, they may serve other functional roles that warrant future investigation.

SnoKARR Detects snoRNA-mRNA and snoRNA-ncRNA Interactions.

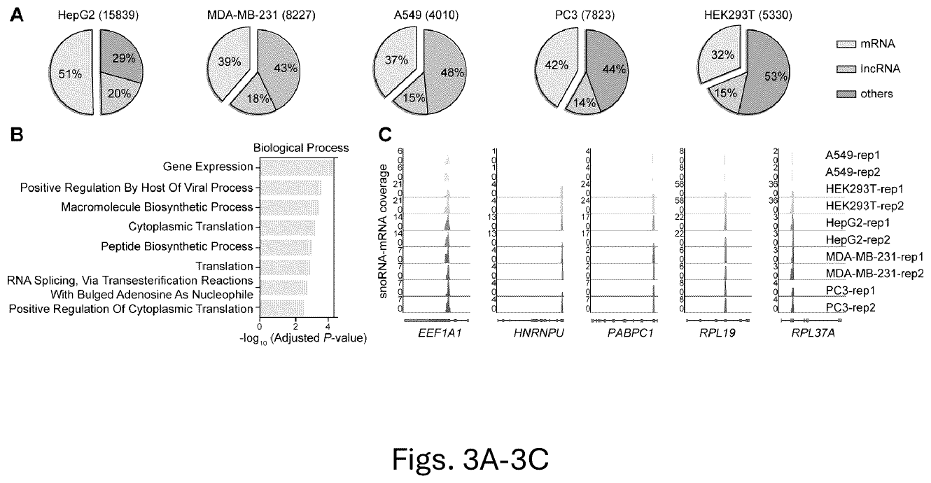

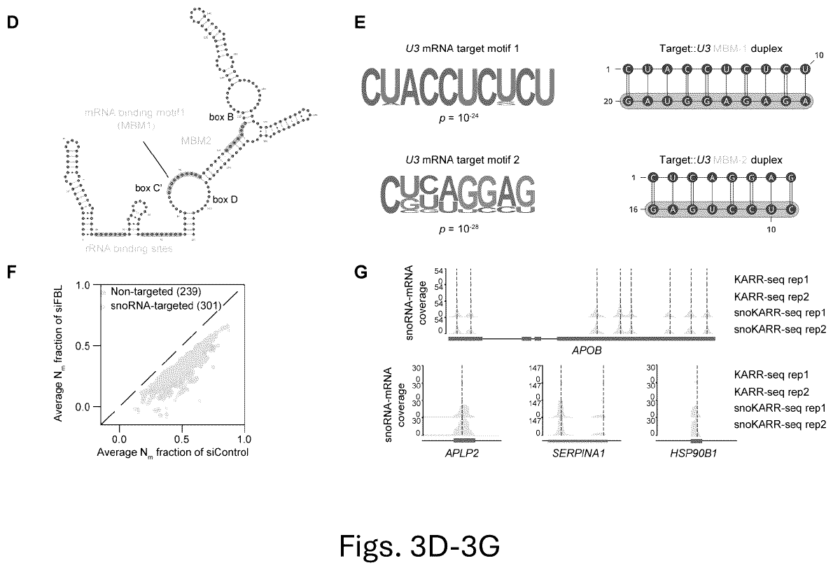

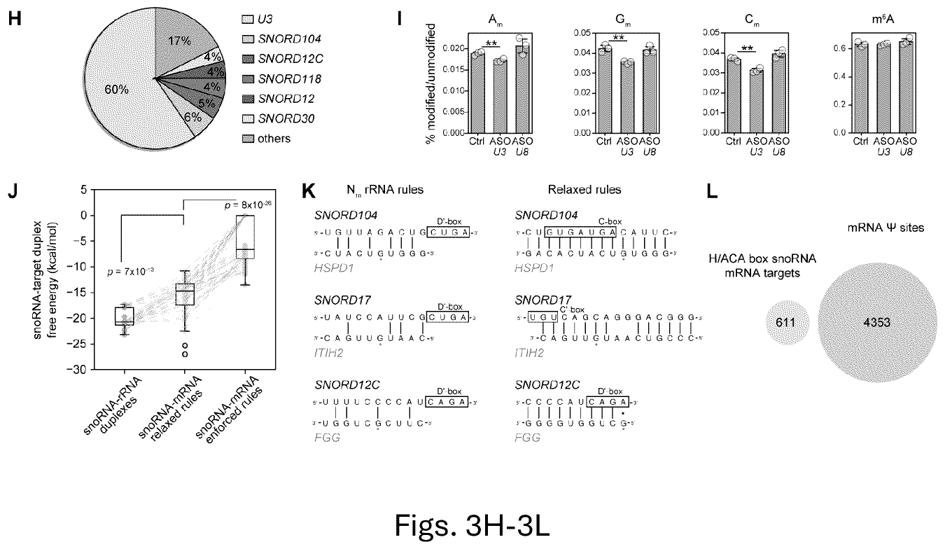

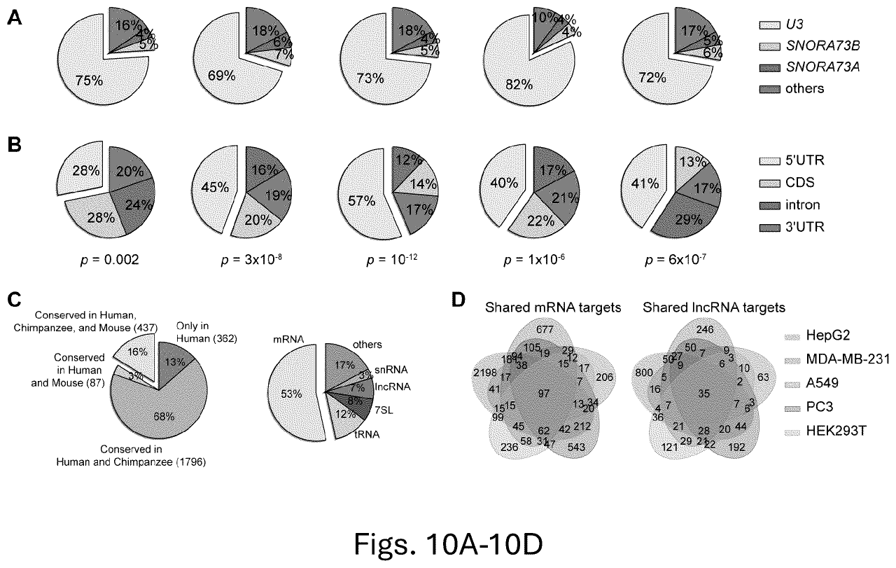

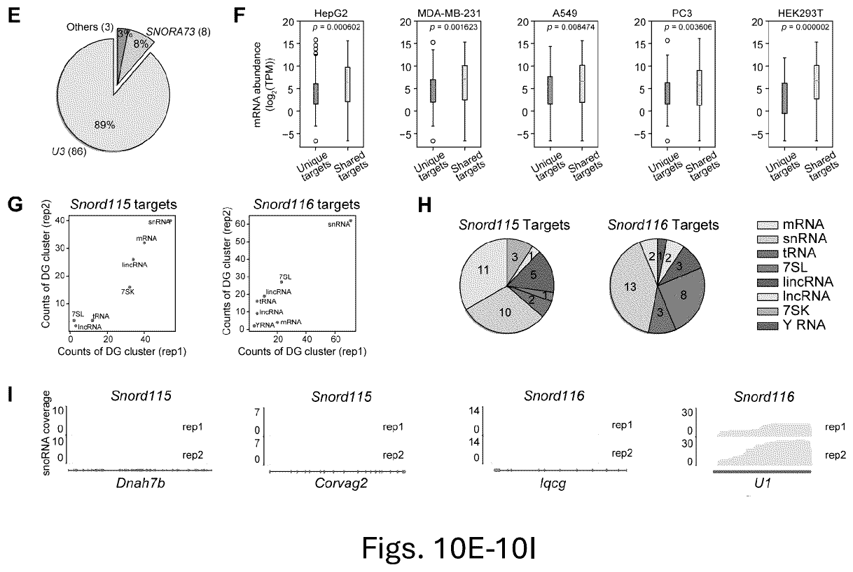

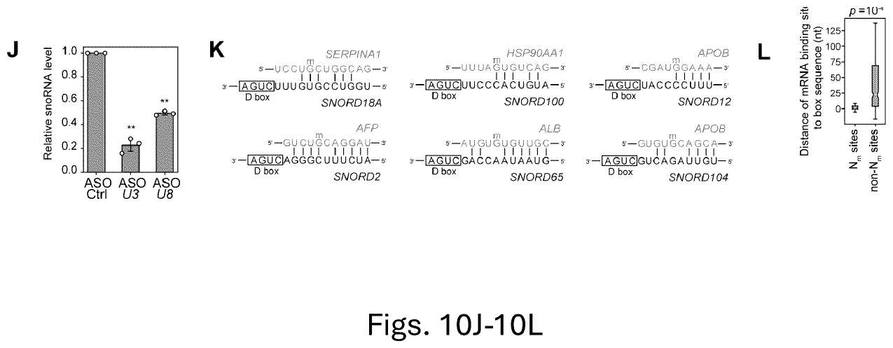

Using snoKARR-seq, the inventors identified thousands of snoRNA targets beyond rRNA across different cell lines (HepG2, MDA-MB-231, A549, PC3, HEK293T). Among these cell lines, mRNA (~30-50%) and lncRNA (~15-20%) were the most abundant targets (FIG. 3A). U3 (~70-80%) and SNORA73A/B (~10%) had particularly high numbers of targets (FIG. 10A), likely attributed to their high abundance in these cell lines (FIG. 8C). For enriched snoRNAs, mRNA targets were slightly enriched in the 5′ untranslated region (5′ UTR) (FIG. 10B) but also abundant in the protein-coding sequences (CDS), 3′ UTR, and introns. Approximately 16% of these snoRNA-mRNA/lncRNA interactions were predicted to be conserved in chimpanzees and mice (FIG. 10C). The inventors detected 97 snoRNA-mRNA and 35 snoRNA-lncRNA interactions consistently across all cell lines (FIG. 10D), representing only a small fraction of targets in each cell line. These results suggest that snoRNA targets may exhibit considerable cell type specificity.

Conserved snoRNA-mRNA interactions predominantly involve U3 (SNORD3A, SNORD3C, SNORD3B-2 etc.) and SNORA73A/B (FIG. 10E). Additionally, mRNA targets shared across all cell lines are significantly more abundant than those not shared, suggesting that abundant snoRNAs may regulate fundamental biological processes through these conserved interactions (FIG. 10F). Gene Ontology (GO) analysis of conserved U3 targets revealed enrichment in terms such as “Gene expression,” “Cytosolic Translation,” and “Biosynthetic processes.” (FIG. 3B). These terms are associated with mRNA targets involved in protein translation (FIG. 3C), indicating that U3 may regulate translation by interacting with these target mRNAs.

U3 is well-known for its essential role in pre-rRNA processing, where it base pairs with the external transcribed spacer of pre-rRNA via its 5′ motif (FIG. 3D)⁵⁹. Interestingly, sequence motif analysis⁶⁰ of the conserved U3 mRNA targets identified two potential mRNA binding motifs (MBM): 5′-CUACCUCUCU-3′ and 5′-CUCAGGAG-3′ (FIG. 3E), which differ from the pre-rRNA binding regions (FIG. 3D), indicating potential non-canonical functions for U3-mRNA interactions.

The inventors also used snoKARR-seq to identify disease-related snoRNAs in mouse brain samples. PWS is a neurogenetic disorder causally linked to defects in imprinted gene dosage at the human chromosomal domain of 15q11.2-13.3^{61,62}. In most PWS cases, clusters of SNORD115/116 genes are the principal genetic determinant, but their targets remain largely unknown. The inventors designed ASOs to enrich multiple copies of Snord115/116 in the mouse brain cortex and identified 43 potential targets (FIG. 10H-I) including mRNA, snRNA, and tRNAs (FIG. 10G-I). Further validation and functional studies of these targets could provide valuable insights into the mechanisms underlying these disease-associated snoRNAs.

snoKARR Detects snoRNA-mRNA Interactions that Lead to Nₘ Modification.

The C/D-box snoRNP has been reported to guide mRNA Nₘ modifications^{19,20}, making snoRNA-mRNA interactions ideal benchmarks to validate the capability of snoKARR-seq to identify mRNA targets. The inventors analyzed snoKARR-seq data from HepG2 cells, as a previous study mapped FBL-dependent Nₘ sites in HepG2 mRNA¹⁹. The inventors found that 56% (301 sites) of the FBL-sensitive Nₘ sites overlap with snoRNA targets identified in snoKARR-seq (FIG. 3F). Notably, the Nₘ sites on mRNA are positioned almost precisely at the center of the peaks in snoRNA-mRNA interaction profiles (FIG. 3G).

Next, the inventors aimed to assign individual mRNA Nₘ sites to corresponding guide snoRNAs. Interestingly, many mRNA Nₘ sites were targeted by multiple snoRNAs, suggesting potential cooperative action in guiding mRNA Nₘ modification, akin to some rRNA Nₘ sites 3. U3 exhibited the highest number of mRNA Nₘ target sites. While U3 is known to interact with pre-rRNA⁶³, the findings indicate its involvement in guiding mRNA Nₘ modification. ASO-mediated knockdown (KD) of U3 in HepG2 cells (FIG. 10J) resulted in a global reduction of mRNA Nₘ levels but not N⁶-methyladenosine (m⁶A) levels, as measured by liquid chromatography-tandem mass spectrometry (LC-MS/MS). In contrast, KD of SNORD118 (U8), which targets only a small subset of Nₘ-modified mRNA, did not alter global mRNA Nₘ levels (FIG. 3H-I). These results confirm that snoRNA, including U3, mediate mRNA Nₘ modification, consistent with previous reports^{19,20}.

The inventors then investigated whether the rules governing snoRNA-guided Nₘ modification in mRNA resemble those in rRNA, where the Nₘ site typically lies 5-nt upstream of the D box^{14,58}. While some snoRNA-mRNA interactions may adhere to this rule (FIG. 10K), many did not form stable duplex structures when constrained by the rRNA rule (FIG. 3J). Conversely, stable duplexes formed when disregarding the rRNA rule, with the Nₘ sites located near or within the C/D-box region (FIG. 3K). These observations indicate that snoRNA-mRNA interactions may follow more flexible binding rules compared to snoRNA-rRNA interactions.

To distinguish snoRNA-mRNA interactions that lead to Nₘ from those that do not, the inventors compared the distances between mRNA binding sites and the box sequences on C/D-box snoRNAs. SnoRNA binding sites on Nₘ-modified mRNAs are closer to the box sequences compared to those on non-modified mRNAs (FIG. 10L). This proximity may reflect the tendency of FBL to bind near the box regions^{64,65}. These results indicate that the binding position of snoRNAs may dictate whether an interaction results in Nₘ modification.

Unlike FBL and C/D-box snoRNA, which form the catalytic complex for Nₘ installation, Ψ in mRNAs can be modified by 13 putative PUS enzymes⁶⁶. DKC1 is one of these enzymes that rely on the H/ACA-box snoRNA to guide rRNA pseudouridylation, while other PUS enzymes act as stand-alone enzymes. Although the inventors detected previously reported snoRNA-rRNA interactions that facilitate rRNA pseudouridylation (FIG. 2A, 9A), the inventors found no overlap between mRNA targets of the 14 abundant H/ACA-box snoRNAs and mapped mRNA Ψ sites in HepG2 cells (FIG. 3L)⁶⁷. The inventors speculated that some of these H/ACA-box snoRNAs may target mRNA with functions beyond Ψ modification.

SNORA73 targets mRNA through a non-canonical motif

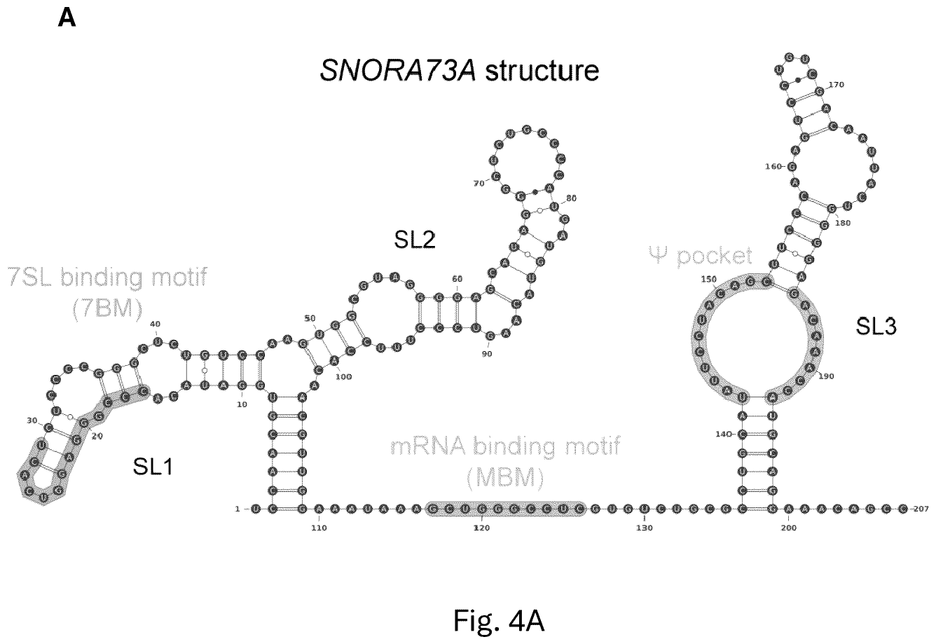

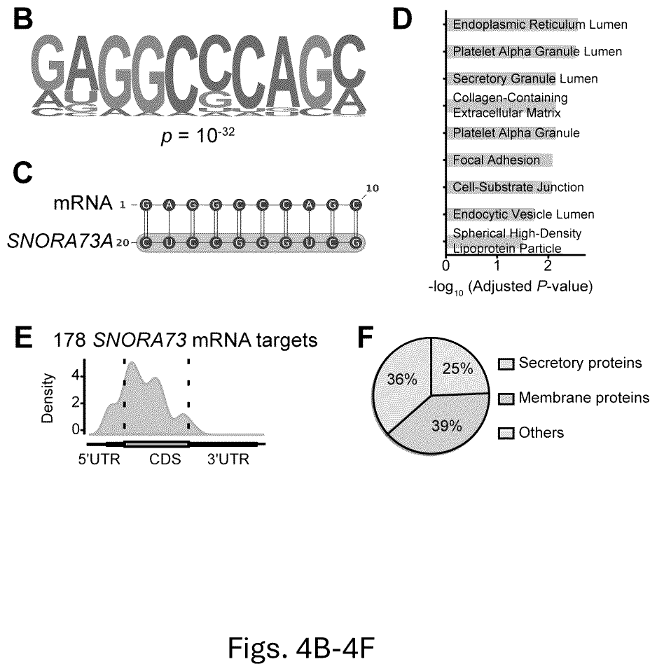

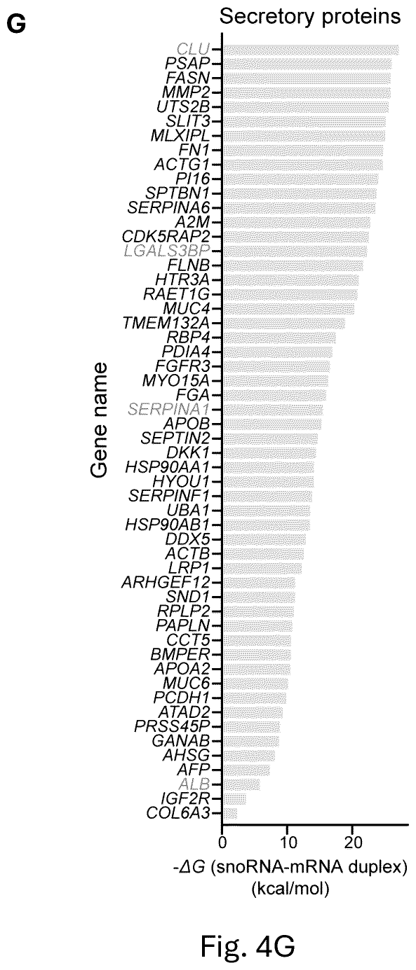

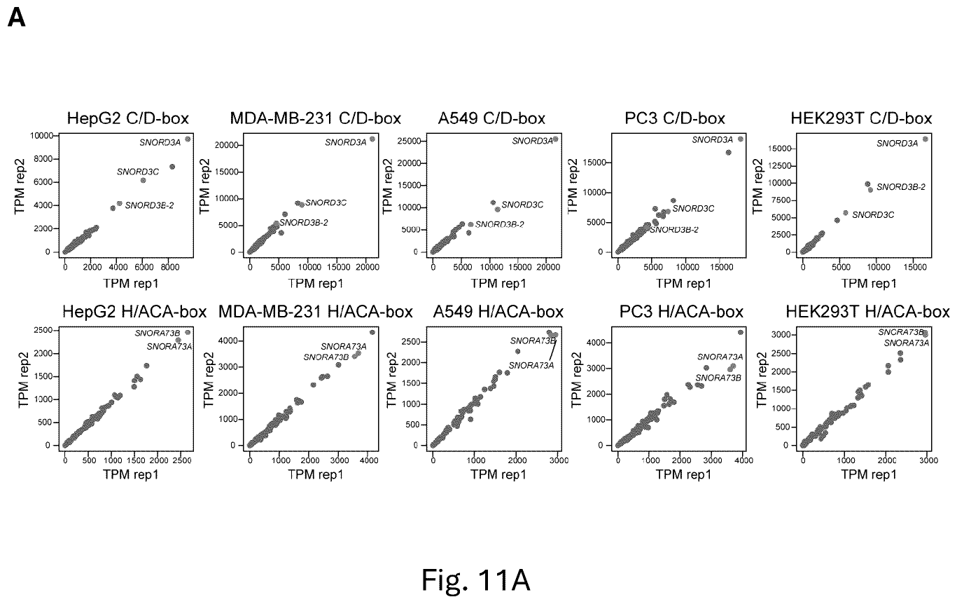

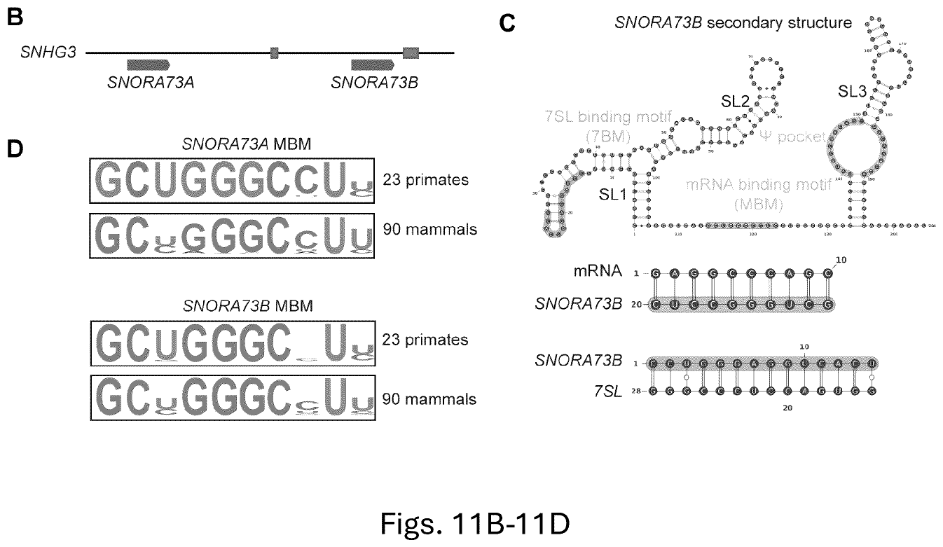

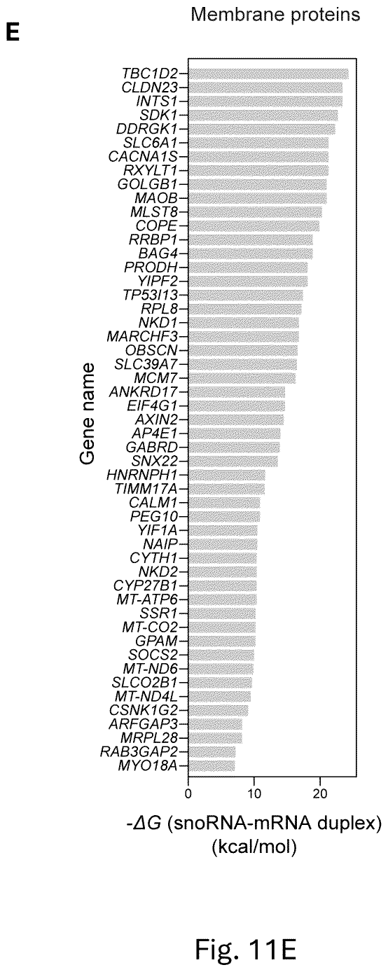

To explore potential non-canonical functions of abundant H/ACA-box snoRNAs in HepG2 cells, the inventors analyzed their sequences and mRNA interactions. The inventors focused on SNORA73, one of the most abundant H/ACA-box snoRNAs (FIG. 11A). The human genome encodes two copies, SNORA73A and SNORA73B, with 95% sequence similarity, both located in introns of the small nucleolar RNA host gene 3 (SNHG3) (FIG. 11B). While H/ACA-box snoRNAs typically adopt a hairpin-hinge-hairpin structure (FIG. 1A), SNORA73 exhibits an atypical configuration. It features only a canonical stem-loop at its 3′ end (SL3 in FIG. 4A and FIG. 11C) containing the canonical Ψ pocket, but the 5′ region comprises an unconventional three-way junction motif (SL1 and SL2). The two stem-loops are connected by a long, unstructured hinge region containing the H box (FIG. 4A, 11C).

Although SNORA73 has not been shown to mediate rRNA modification, yeast SNORA73 could interact with rRNA through the canonical Ψ pocket via base pairing^{68,69}. To investigate SNORA73-mRNA interactions, the inventors performed sequence motif analysis⁶⁰ on SNORA73 mRNA targets. Intriguingly, the inventors identified a 10-nt consensus sequence motif 5′-GAGGCCCAGC-3′ (FIG. 4B) that does not base pair with the canonical Ψ pocket (FIG. 4A, 11C). Instead, this motif forms a perfect 10-base pair (bp) Watson-Crick duplex (FIG. 4C, 11C) with a complementary sequence in the unstructured hinge region of SNORA73A B (5′-GCUGGGCCUC-3′, FIG. 4A, 11C), which the inventors termed the mRNA-binding motif (MBM). Notably, the MBM is conserved across mammals (FIG. 11D). Additionally, the inventors found no overlap between SNORA73 mRNA targets and previously reported mRNA Ψ sites (FIG. 3L), suggesting that SNORA73 binds to mRNA via the MBM, independent of the canonical Ψ pocket.

SNORA73 Targets mRNAs Encoding Secretory Proteins and Facilitates their Secretion.

To explore the biological functions of SNORA73-mRNA interactions, the inventors conducted GO analysis using data from HepG2, a liver cell line. SNORA73 mRNA targets showed a strong preference towards ER and granule lumens (FIG. 4D). These targets contain potential base-pairing sequences with the SNORA73 MBM, which are not limited to their coding regions (FIG. 4E). Approximately 60% of these mRNA targets encode either secretory proteins or membrane proteins (FIG. 4F), which necessitate co-translational ER import and processing for translocation to the plasma membrane. The free energy of SNORA73 MBM-target duplexes (ΔG_{duplex}) ranges from −3 to −30 kcal/mol (FIG. 4G, 11E). Given that the liver is the primary organ for synthesizing secreted proteins and is responsible for 85-90% of circulating proteins in the blood⁷⁰, the inventors postulate that SNORA73 may play a role in protein translocation and plasma membrane delivery by targeting mRNAs encoding secretory or membrane proteins.

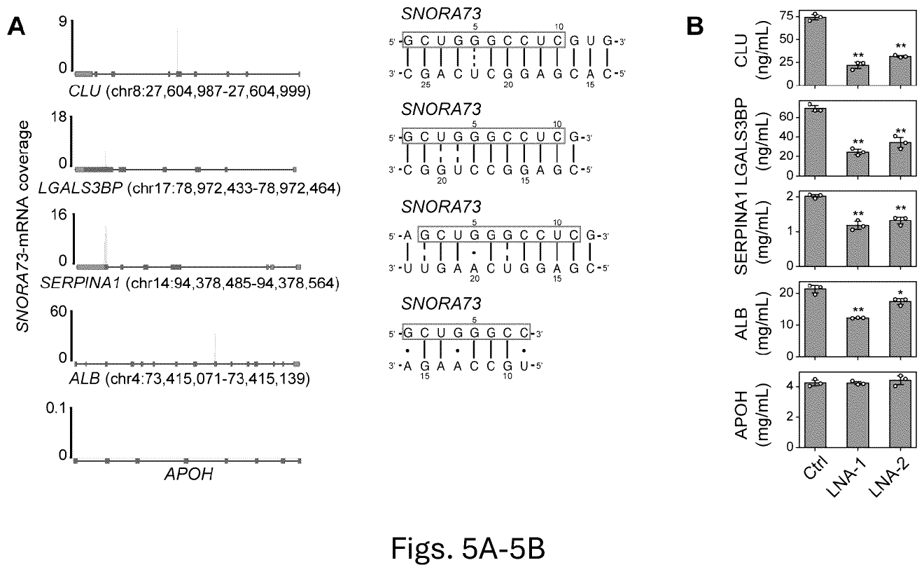

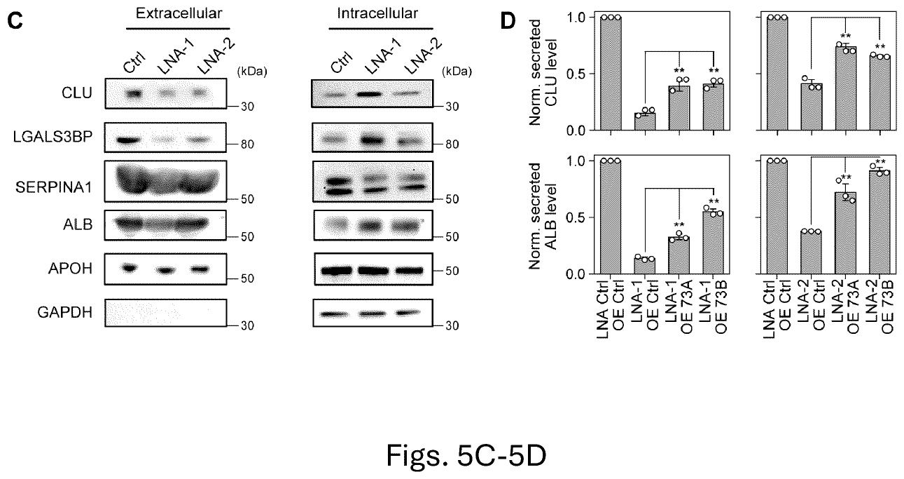

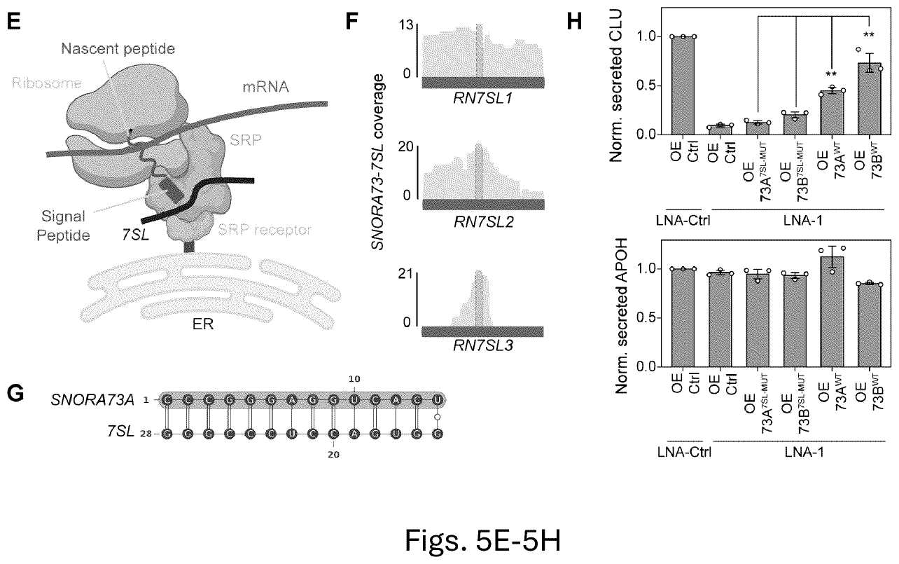

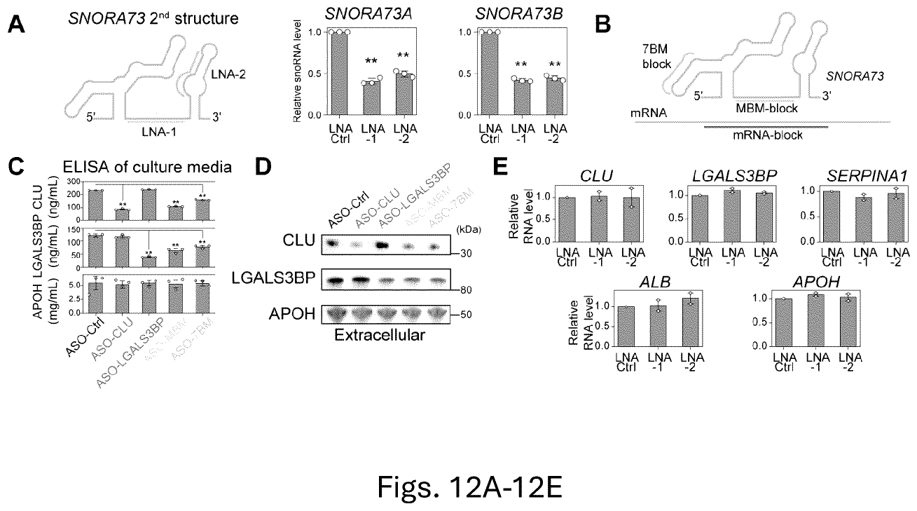

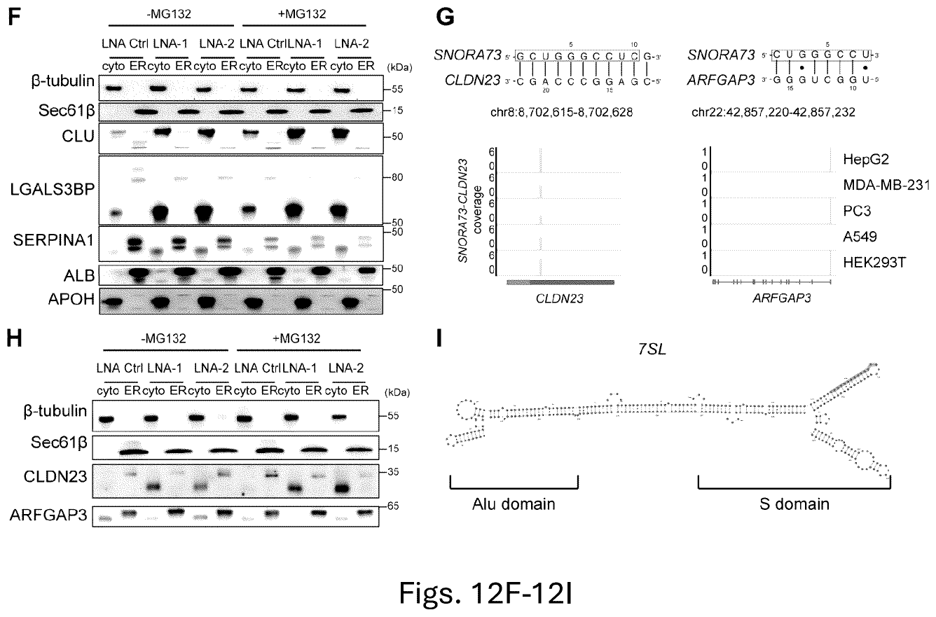

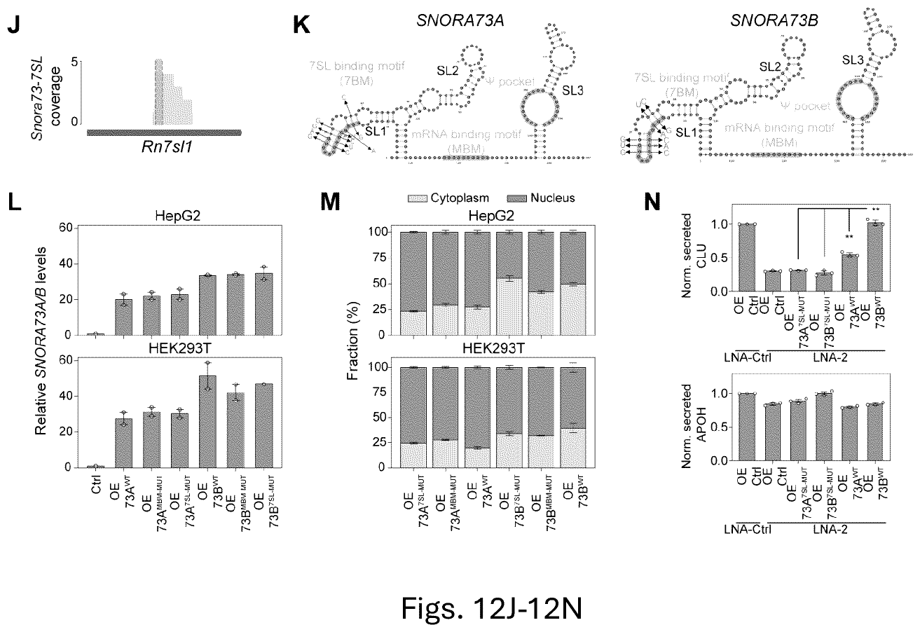

To test this hypothesis, the inventors assessed the impact of SNORA73 on the secretion of several proteins whose mRNAs are SNORA73 targets (FIG. 5A). The inventors selected clusterin (CLU), galectin 3 binding protein (LGALS3BP), alpha antitrypsin (SERPINA1), and albumin (ALB), representing a range of ΔG_{duplex} values (FIG. 4G). The inventors used two locked nucleic acid (LNAs) to KD SNORA73 (FIG. 12A) and evaluated the protein secretion levels in the cell culture media using enzyme-linked immunosorbent assay (ELISA) (FIG. 5B) and Western blot (FIG. 5C). KD of SNORA73 reduced the secretion of all four target proteins, while the non-target protein apolipoprotein H (APOH) remained largely unaffected (FIG. 5B-C). Additionally, 2′-MOE (methoxyethyl)-based steric blocking of SNORA73 MBM and snoRNA binding sites on mRNAs led to reduced protein secretion (FIG. 12B-D). Restoring SNORA73 in KD cells reversed the secretion defects, confirming the role of SNORA73 in regulating protein secretion (FIG. 5D).

The inventors found that the mRNA levels of these secretory proteins remained unchanged (FIG. 12E) upon SNORA73 KD, indicating that reduced protein secretion was unlikely due to the altered mRNA abundance. While the intracellular levels of CLU, LGALS3BP, and ALB increased, SERPINA1 levels decreased upon SNORA73 KD (FIG. 5C), suggesting that the reduction in secretion may not directly associate with changes in translation. This implies that SNORA73-mRNA interactions may facilitate nascent protein translocation rather than promoting translation. To further investigate the role of SNORA73 in intracellular protein distribution, the inventors analyzed cytosolic and ER fractions in control and SNORA73 KD cells. Cytosolic levels of CLU, LGALS3BP, SERPINA1, and ALB increased upon SNORA73 KD, both with and without proteasome inhibition by MG132 (FIG. 12F). In contrast, their ER levels slightly decreased upon SNORA73 KD (FIG. 12F). As expected, the cytosolic and ER APOH levels remained largely unchanged following SNORA73 KD (FIG. 12F). These results support the role of SNORA73 in promoting the translocation of target proteins from the cytosol into the ER.

The inventors also examined the impact of SNORA73 on membrane protein translocation. Upon SNORA73 KD, cytosolic levels of CLDN23 (a tight junction membrane protein, FIG. 12G) increased both with and without proteasome inhibition, while its ER levels decreased in the presence of MG132 (FIG. 12H). In contrast, SNORA73 KD did not increase cytosolic levels of ARFGAP3 (a Golgi-associated membrane protein, FIG. 12G), whose membrane insertion is independent of SRP⁷¹ (FIG. 12H). These findings suggest that SNORA73 plays a role in mediating the translocation of both secretory and membrane proteins.

SNORA73 Mediates Protein Translocation to ER Via mRNA-snoRNA-7SL Interactions.

Next, the inventors explored how snoRNA-mRNA interactions could facilitate protein secretion. In the canonical co-translational translocation model (FIG. 5E), the growing peptide chain interacts with the SRP ribonucleoprotein complex through signal peptides. The ribosome-nascent-peptide-SRP complex then docks onto the ER membrane through interactions between SRP and SRP receptor, further facilitated by ribosome binding to the translocation channel complex⁷². Subsequently, the nascent polypeptide elongates directly from the ribosome tunnel into the membrane channel, driven by GTP hydrolysis^{37,38}. Although signal-peptide-independent translocation pathways have been reported^{40-42,44}, the presence and roles of non-protein signals in these pathways remain unclear.

Interestingly, snoKARR-seq data revealed a 14-bp duplex between SNORA73 and the helix 6 in the S domain of 7SL RNA (FIG. 5F-G, 4A, 11C, 12I), the RNA component of the SRP. This interaction occurs through the sequence in SL1 of SNORA73, which the inventors termed the 7SL-binding motif (7BM), distinct from the canonical Ψ pocket and the MBM (FIG. 4A, 11C). This SNORA73-7SL interaction is conserved in mice (FIG. 12J). Given that SNORA73 interacts with both mRNA and 7SL RNA via the MBM and 7BM, respectively, the inventors hypothesize that these ternary RNA interactions involving mRNA-SNORA73-7SL may modulate protein translocation to the ER and subsequent secretion. In this model, SNORA73 acts as a “molecular glue”, linking the SRP complex with target mRNAs through dual RNA-RNA interactions (snoRNA-mRNA and snoRNA-7SL, FIG. 4C, 5G, 11C).

To test this hypothesis, the inventors mutated the 7BM on SNORA73 while preserving the secondary structure of SNORA73 (FIG. 12K) and introduced either the mutant (SNORA73^{SL-MUT}) or wild-type (WT) SNORA73A/B (SNORA73^{WT}) into SNORA73 KD cells. The inventors then assessed their ability to restore protein secretion following SNORA73 KD. The 7BM mutation did not affect the expression or localization of SNORA73 (FIG. 12L-M). KD of SNORA73 decreased CLU secretion to −10%-30% compared with control cells. While the reintroduction of SNORA73^{WT} partially restored protein secretion, SNORA73^{SL-MUT} led to significantly less restoration (FIG. 5H, 12N). Additionally, steric blocking of SNORA73 7BM led to decreased protein secretion (FIG. 12B-D), indicating the importance of the 7BM in SNORA73 for effective protein secretion.

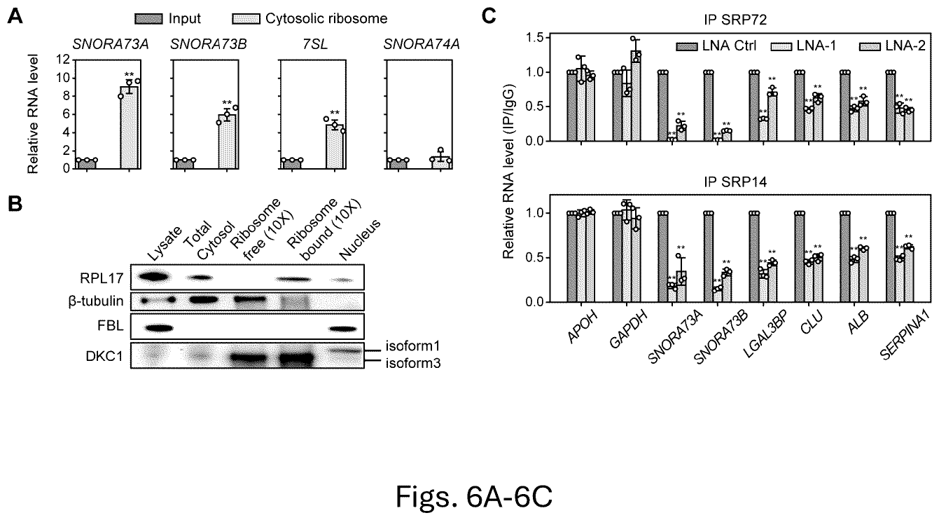

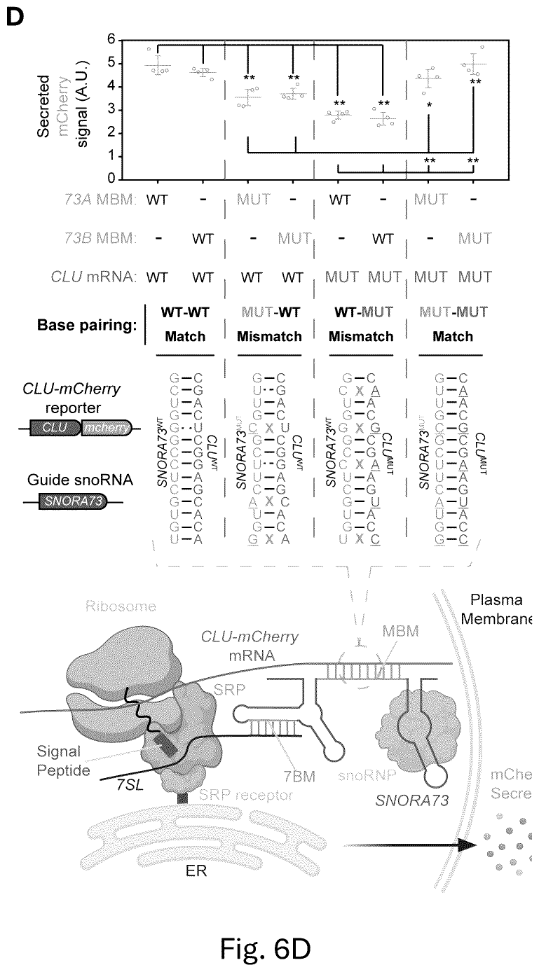

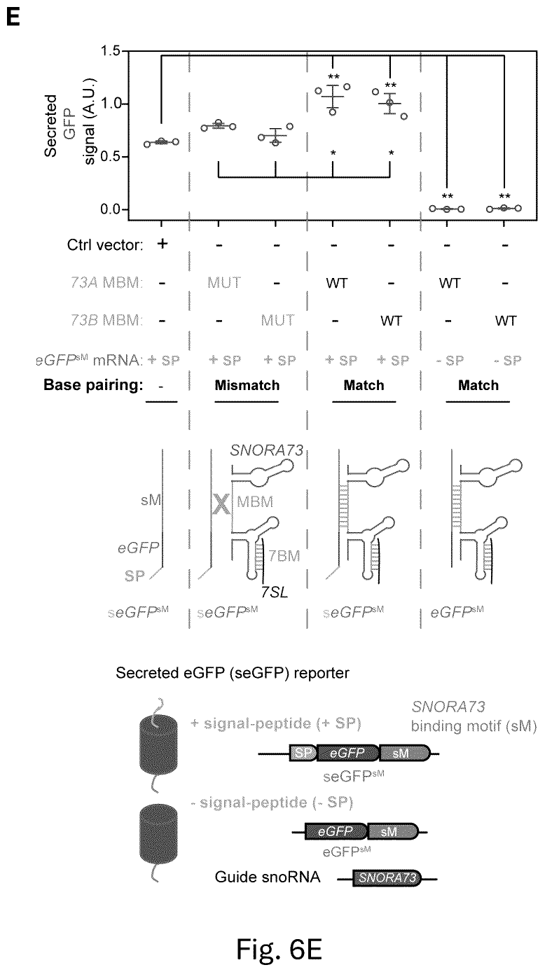

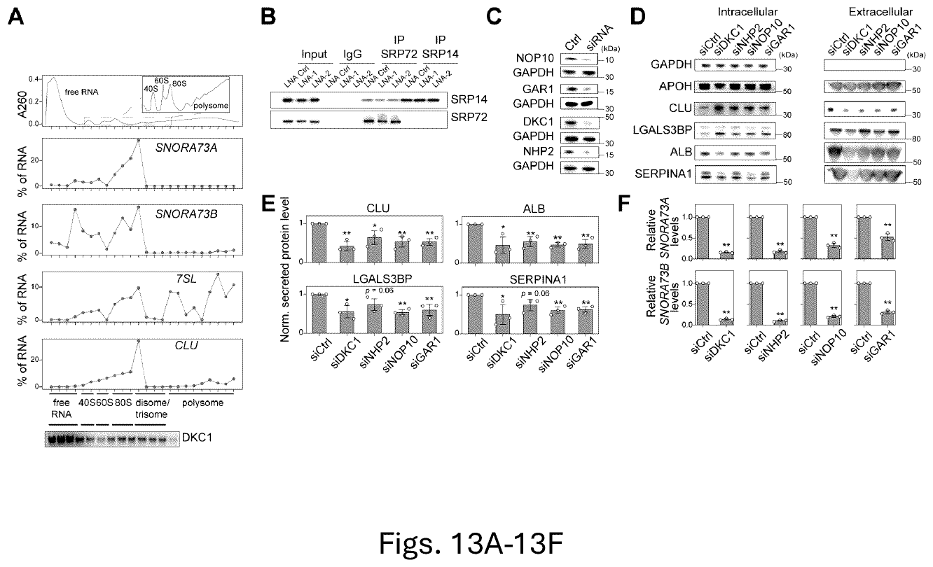

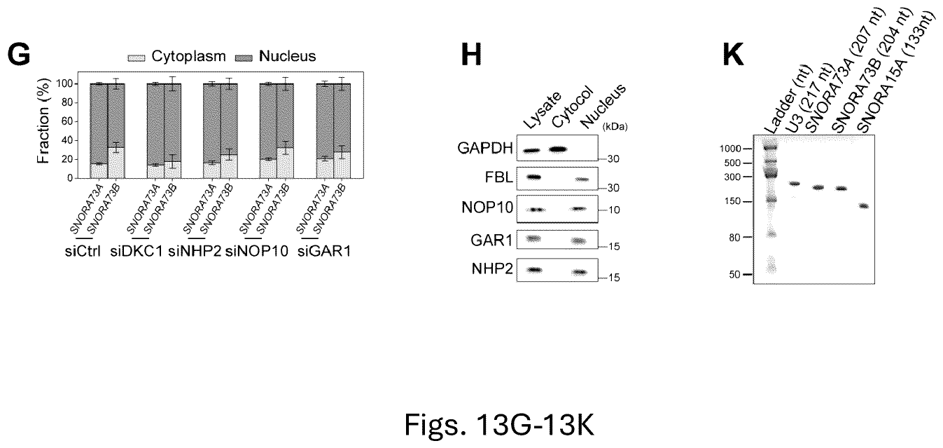

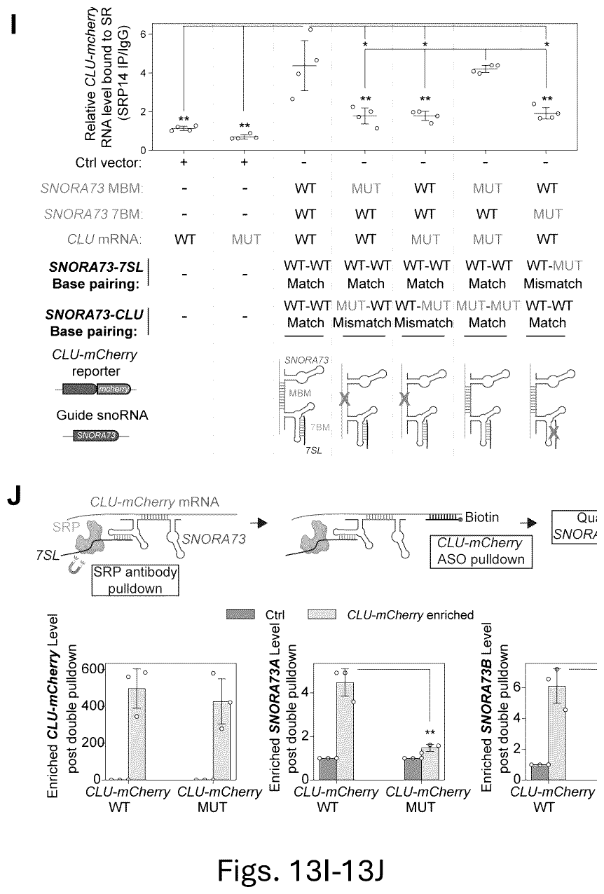

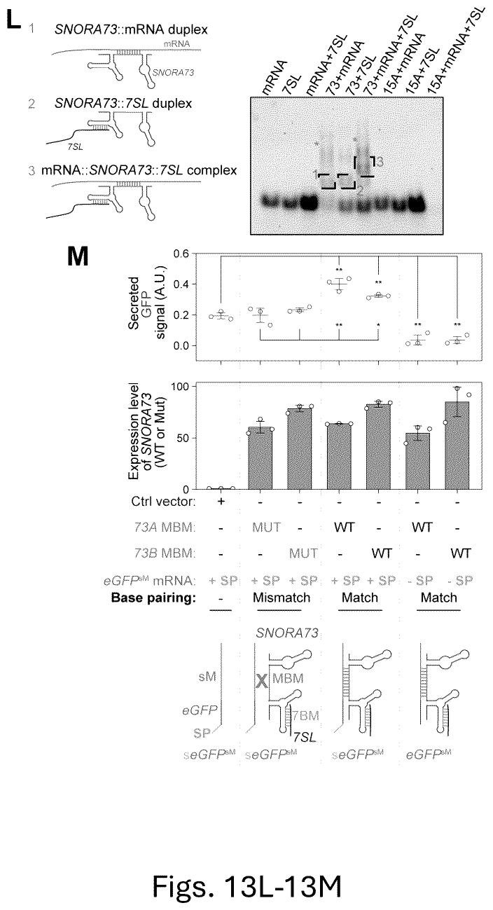

The anticipated mRNA-SNORA73-7SL interactions during co-translational protein translocation would predict an enrichment of SNORA73 and its canonical protein binding partners, such as DKC1, in cytosolic ribosome complexes. Indeed, the inventors observed enriched SNORA73, 7SL, and DKC1 but not the control non-7SL binding snoRNA (SNORA74A) in the cytosolic ribosome fraction, confirming the association of SNORA73 snoRNP with cytosolic ribosomes (FIG. 6A). Notably, DKC1 isoform-3, which lacks the C-terminal nuclear localization signals (NLS) 73, was enriched in the cytosolic ribosome fraction, whereas the full-length DKC1 (isoform-1) was restricted to the nucleus (FIG. 6B). Moreover, SNORA73, 7SL, and CLU mRNA colocalize in the monosome and low-polysome fractions (FIG. 13A). Since SRP engages with N-terminal signal peptides at the ribosome's exit tunnel at the monosome stage, causing translational arrest until it binds the SRP receptor on the ER membrane, this colocalization suggests that SNORA73 may dissociate from the ribosome early in translation.

If SNORA73-mRNA interactions affect protein translocation to the ER by promoting SRP binding, the inventors expect SNORA73 to enhance SRP recruitment to the target mRNA. The inventors performed SRP immunoprecipitation (IP) using antibodies against SRP14 and SRP72 (FIG. 13B), two of the SRP subunits, followed by RT-qPCR to quantify the SRP-bound mRNA levels in control and SNORA73 KD cells. SNORA73 KD resulted in reduced SRP binding to SNORA73-target mRNAs, while SRP binding to non-target mRNAs remained unchanged (FIG. 6C). These results demonstrate that SNORA73 promotes SRP recruitment to target mRNAs, likely through mRNA-snoRNA-7SL ternary interactions.

Next, the inventors assessed the impact of the canonical H/ACA-box snoRNA binding proteins (DKC1, NHP2, NOP10, and GAR1) on the SNORA73-mediated protein secretion. Depletion of these proteins reduced both SNORA73 levels and protein secretion but had minimal effect on SNORA73 localization (FIG. 13C-G). These nuclear proteins (FIG. 13H), including the nuclear isoform of DKC1 (FIG. 6B), may function as scaffolds, stabilizing H/ACA-box snoRNAs during their biogenesis in the nucleus. However, the cytosolic isoform of DKC1 is more likely directly involved in protein translocation through the pathway (FIG. 6B).

The mRNA-SNORA73-7SL Triad is Modular and can be Engineered for Protein Secretion.

If SNORA73 functions as a molecular glue to direct target mRNAs encoding secretory proteins to the SRP complex, these RNA-RNA interactions should be modular and programmable. To test this, the inventors designed a reporter assay (FIG. 6D). Specifically, CLU mRNA was fused with mCherry (CLU^{WT}-mCherry) to allow measurement of CLU secretion via mCherry fluorescence in the cell culture media. The inventors constructed a synonymous mutant of CLU mRNA (CLU^{MUT}-mCherry) with a disrupted snoRNA-binding sequence (FIG. 6D). The inventors also constructed SNORA73 mutants (SNORA73^{MUT}) with an altered MBM to base pair with CLU^{MUT}-mCherry (FIG. 6D). The inventors co-expressed either CLU^{WT}-mCherry or CLU^{MUT}-mCherry with the corresponding SNORA73 constructs and monitored mCherry fluorescence in the cell culture media to evaluate the impact of snoRNA-mRNA interactions on protein secretion. Disrupting base pairing by co-expressing SNORA73^{MUT} with CLU^{WT}-mCherry, or SNORA73^{WT} with CLU^{MUT}-mCherry (“Mismatch” in FIG. 6D), reduced protein secretion by ~15-45% compared to the co-expression of SNORA73^{WT} with CLU^{WT}-mCherry (FIG. 6D). Conversely, co-expression of SNORA73^{MUT} with CLU^{MUT}-mCherry, where the snoRNA-mRNA base pairing was restored (“Match” in FIG. 6D), did not alter protein secretion. These results demonstrate that snoRNA-mRNA base-pairing is critical for efficient protein secretion.

To further validate the formation of the mRNA-snoRNA-7SL triad, the inventors first quantified CLU transcript levels bound by SRP using RIP-qPCR and the reporter assay. Disruption of either the SNORA73-7SL or SNORA73-mRNA duplex reduced SRP-bound CLU transcripts, while restoring duplex complementarity reversed this effect (FIG. 13I). Next, the inventors performed a “double pulldown” experiment by overexpressing CLU-mCherry and pulling down SRP-bound RNA via IP, and subsequently capturing CLU mRNA using ASOs. The inventors observed a ~4.5-fold enrichment of SNORA73 in the group with consecutive SRP and CLU pulldown compared to a control group with consecutive SRP and GFP pulldown (FIG. 13J). Disrupting the SNORA73-mRNA duplex reduced SNORA73 enrichment in this assay (FIG. 13J). Lastly, the inventors in vitro transcribed SNORA73B (FIG. 13K) and annealed it with either a fluorescein-labeled mRNA fragment containing the SNORA73 binding sequence, a fluorescein-labeled 7SL RNA fragment containing the SNORA73 binding sequence, or both. SNORA73B, but not the control SNORA15A (FIG. 13L), formed dimers with either mRNA or 7SL RNA (FIG. 13L), as evidenced by upshifted RNA bands in the native RNA gel. The combination of SNORA73B, mRNA, and 7SL together resulted in a further upshifted band compared to the dimers, indicating the formation of the mRNA-SNORA 73-7SL heterotrimer (FIG. 13L). In contrast, the combination of mRNA and 7SL without SNORA73B did not lead to dimer formation, indicating the critical “gluing” role of SNORA73B.

Given the modularity of the snoRNA-mRNA interactions, snoRNA sequences can be engineered to guide the secretion of arbitrary proteins. The inventors tested this hypothesis using a secreted eGFP (seGFP) reporter (FIG. 6E) by adding a signal-peptide sequence to the 5′ end of eGFP mRNA to drive secretion 74. The inventors further introduced the SNORA73-binding mRNA motif (sM) to the 3′ end of the eGFP mRNA (seGFP^{M}). The inventors co-expressed the seGFP^{sM} with either SNORA73^{WT} or SNORA73^{MUT} and monitored the GFP fluorescence in the cell culture media to assess secretion levels. Co-expression of SNORA73^{WT} with seGFP^{sM} can enhance its secretion by ~30%-50% compared with co-expression of a control vector and seGFP^{sM} or co-expression of SNORA73^{MUT} and seGFP^{sM} in both WT and SNORA73 KD cells (FIG. 6E, 13M). Notably, removal of the signal-peptide (eGFP^{sM}) significantly reduced secretion, even with SNORA73^{WT} co-expression (FIG. 6E, 13M), indicating that mRNA-snoRNA-7SL interactions alone may not efficiently drive secretion. This RNA signal plays a key role in guiding the secretion of proteins with low signal-peptide efficiency⁷⁵.

### The Model of RNA Signal-Mediated Protein Secretion.

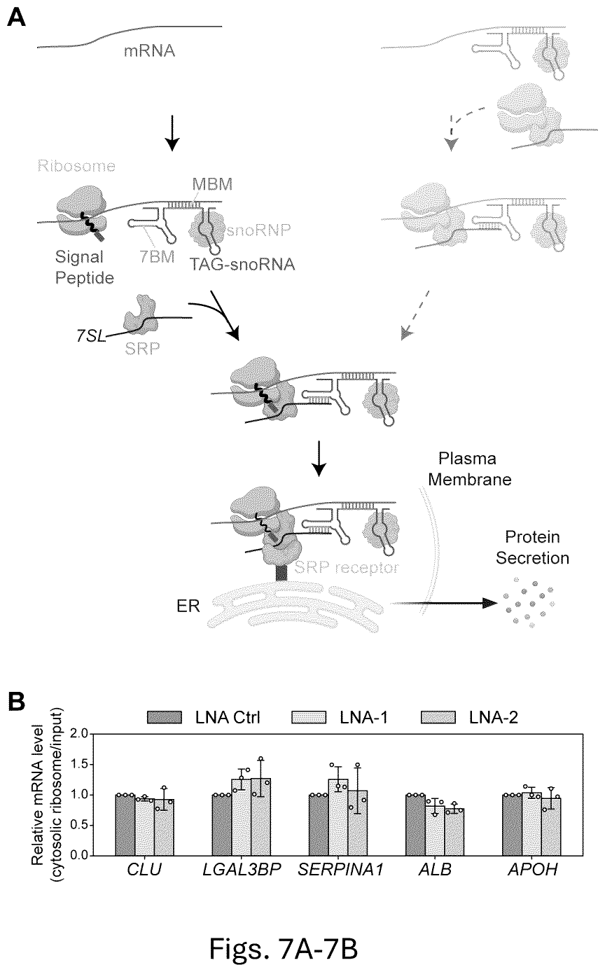

The presence and roles of non-protein signals in protein secretory pathways remain largely unknown. Here, the inventors propose an RNA signal-guided protein translocation mechanism. Specifically, snoRNAs bind to mRNAs encoding secretory/membrane proteins via the MBM, while the 7BM on snoRNA interacts with 7SL. The resulting mRNA-snoRNA-7SL ternary interactions facilitate SRP recruitment to the translation machinery, enhancing the translocation of nascent proteins to the ER (FIG. 7A).

The inventors also investigated whether the mRNA-snoRNA-7SL triad could influence ribosome recruitment to mRNAs, as SRP can bind to non-translating ribosomes lacking nascent peptides⁷⁶ (FIG. 7A, dashed arrows). To test this, the inventors isolated cytosolic ribosome fractions and compared the levels of SNORA73-targeted mRNAs in WT and SNORA73 KD cells. The levels of these mRNAs in cytosolic ribosomes remained largely unchanged upon SNORA73 KD (FIG. 7B), suggesting that SNORA73 does not affect ribosome binding to its target mRNA. These results indicate that the mRNA-snoRNA-7SL interactions specifically promote SRP recruitment after nascent peptide synthesis begins (FIG. 7A).

snoKARR Detects snoRNA-mRNA Interactions that Lead to Nm Modification.

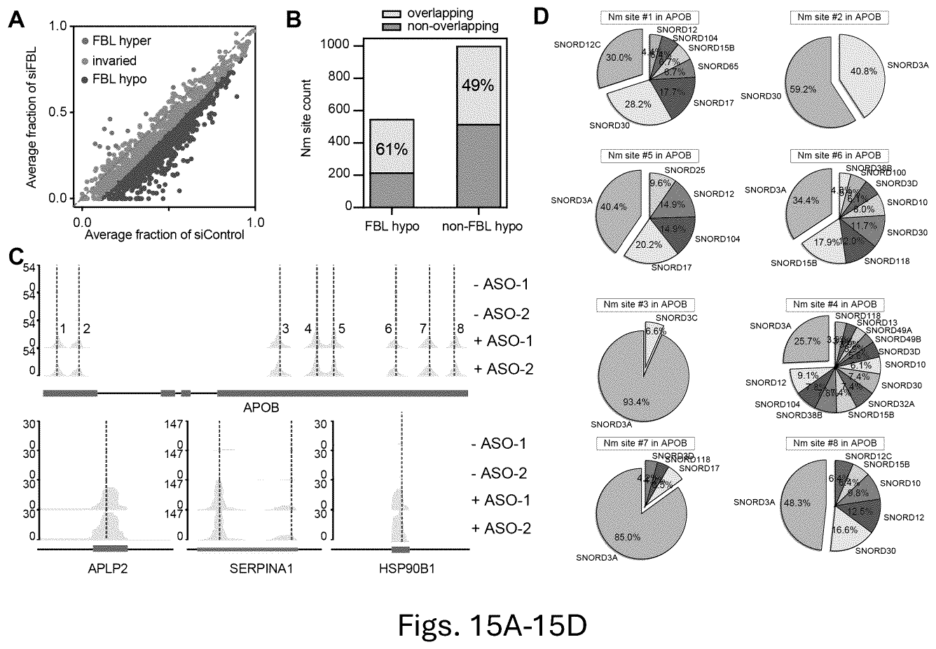

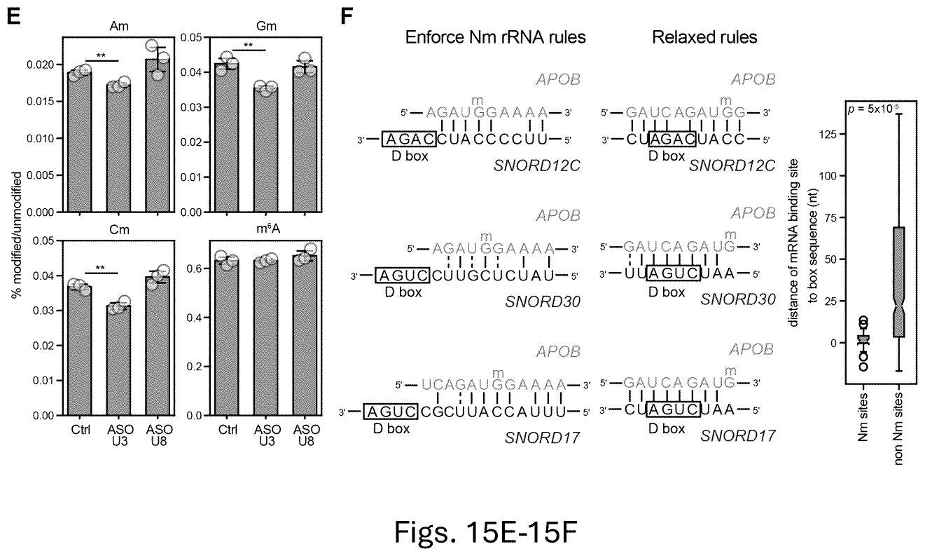

It has been reported that C/D box snoRNP could guide mRNA Nm modification. These snoRNA-mRNA interactions could serve as a benchmark to validate the capacity of snoKARR-seq to identify snoRNA mRNA targets. The inventors chose to analyze snoKARR-seq data generated from HepG2 cells because a previous study has mapped FBL-dependent Nm sites in HepG2 mRNA (FIG. 15A). By overlapping snoRNA mRNA targets and Nm sites, the inventors found that 61% (~300 sites) of the FBL hypomethylated Nm sites and 49% (~500 sites) of the FBL independent Nm sites overlap with snoRNA targets identified in snoKARR-seq (FIG. 15B). Strikingly, the Nm sites on mRNA are positioned at the center of the peaks in snoRNA-mRNA interaction profiles (FIG. 15C).

Next, the inventors sought to assign individual mRNA Nm sites to individual guide snoRNA. Interestingly, the inventors found that Nm sites on mRNA could be targeted by multiple snoRNAs, indicating that different snoRNAs may work cooperatively to guide mRNA Nm modification, which is similar to some of the rRNA Nm sites (FIG. 15D). Additionally, the inventors found that SNORD3A (U3) has the most amount of mRNA Nm site targets. U3 is known to interact with pre-rRNA to regulate pre-rRNA processing but has not been shown to guide rRNA Nₘ modification. The results suggest that U3 could possibly guide Nₘ modification in mRNA. The inventors used ASO to block SNORD3A and quantify the changes of Nm level using mass-spectrometry. The inventors indeed observed a global reduction of Nm level but not m⁶A level only when blocking U3 but not U8 (FIG. 15E). These results suggest that snoKARR-seq can detect snoRNA-mRNA interactions and indicate that snoRNA can indeed guide mRNA Nₘ modification, which is consistent with previous studies(Chen et al., 2023; Elliott et al., 2019).

Finally, the inventors assessed whether the rules of snoRNA-guided Nₘ modification in mRNA is similar to those in rRNA, where the Nₘ modification site is ~4-5 bp away from the D/D′ box. Interestingly, the inventors found that snoRNA-mRNA cannot form reasonably stable duplex structure if enforcing the same rule (FIG. 15F). On the other hand, stable duplex (>7 bp) can be formed if neglecting the rule. The Nₘ sites in these duplexes are close to or involved in D/D′ box region. The binding sites of Nm-harboring mRNAs are more closed to the box sequences comparing to snoRNA-mRNA interactions that do not result in Nm modification (FIG. 15F). This could potentially be explained by the fact that FBL binding sites are closer to the box regions. These observations indicate that the binding rules of snoRNA-mRNA interactions may be more promiscuous than snoRNA-rRNA interactions. The inventors speculate that the difference may stem from different protein binding partners of snoRNA and FBL in mRNA and rRNA, which needs further investigation in the future.

Unlike FBL and C/D box snoRNA, which form the most important catalytic complex that install Nm on mRNA, Ψ in mRNAs can be modified by 13 putative PUS enzymes(Hamma and Ferre-D′ Amare, 2006). DKC1 is one of the PUS enzymes that rely on H/ACA box snoRNA to guide rRNA pseudouridination while other PUS enzymes function as stand-alone enzymes. The inventors did not observe significant overlap between the targets of the 14 H/ACA box snoRNA enriched in snoKARR-seq and Ψ sites mapped using BID-seq(Dai et al., 2023). This is likely because DKC1/snoRNA may not be the main pseudouridine synthase in mRNA as DKC1 KD only needs to reduction of ~30 mRNA Ψ level(Dai et al., 2023), and the inventors only enriched a small number of H/ACA box snoRNAs.

### DISCUSSION

Despite accumulating evidence for snoRNA functions beyond RNA modifications, systematically identifying their cellular targets has been challenging. snoKARR-seq enables the comprehensive and high-throughput identification of snoRNA targets. Using ASO-based cDNA enrichment, the inventors identified over 1,000 snoRNA-mRNA and snoRNA-incRNA interactions across various cell lines and mouse tissues. This versatile approach extends beyond snoRNA-mRNA interactions, as the cDNA enrichment strategy can be adapted to target any RNA species in proximity ligation-based sequencing approaches^{47,50,77-80}.

### SNORA73 as a Molecular Ternary Glue to Facilitate Protein Secretion

SnoKARR-seq revealed that many C/D-box snoRNA targets were found to overlap with mRNA Nₘ sites, suggesting that snoRNA could guide mRNA 2′-O-methylation, consistent with prior studies^{19,20}. However, unlike the strict binding rules observed for rRNA Nₘ sites, the interactions between C/D-box snoRNAs and Nₘ-containing mRNAs appear more flexible, warranting future studies to uncover the mechanisms and implications of mRNA Nₘ modification. In contrast, no overlap was observed between the 14 highly abundant H/ACA-box snoRNA targets and mapped mRNA Ψ sites, suggesting functions beyond RNA modification.

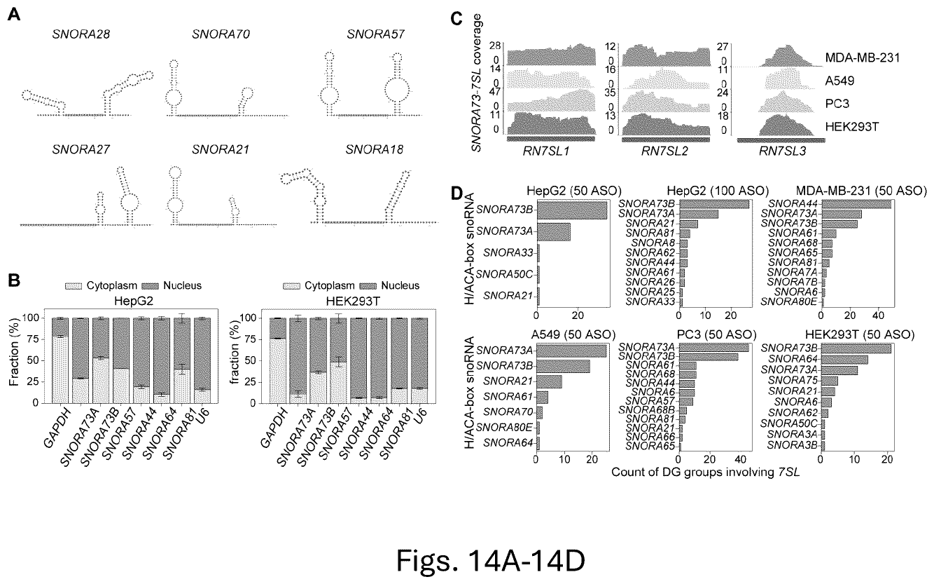

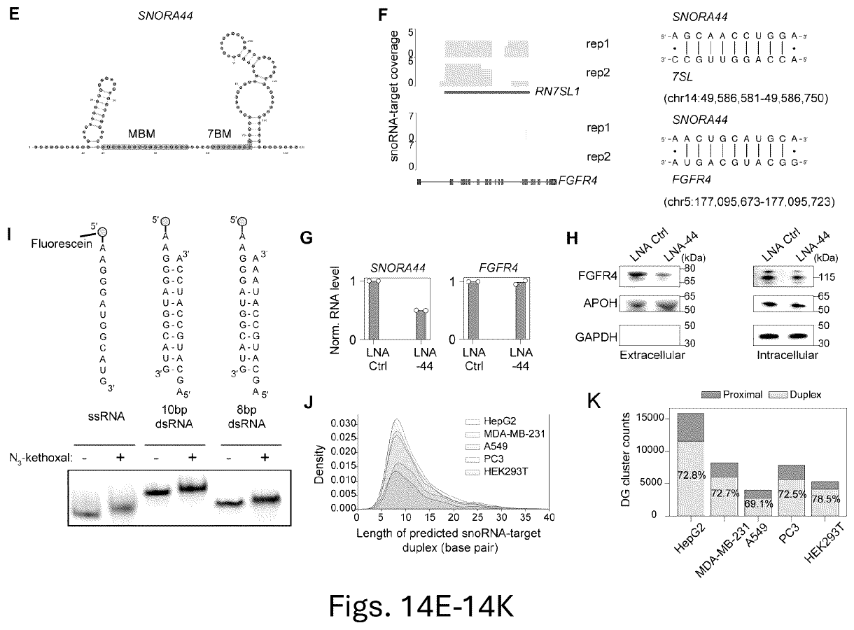

The inventors investigated SNORA73, which features a distinct structure compared to other canonical H/ACA-box snoRNAs. SNORA73 binds mRNA targets through a non-canonical motif (MBM) located in its flexible hinge region. Additionally, the inventors identified several other snoRNAs with similarly unstructured long-hinge motifs (FIG. 14A), accessible for potential intermolecular RNA-RNA interactions. Some of these snoRNAs can localize to the cytoplasm and may participate in mRNA targeting in a manner similar to SNORA73 (FIG. 14B).

SNORA73 targets mRNAs encoding secretory and membrane proteins, and its depletion impairs the secretion of target proteins. This unexpected function of snoRNA in promoting protein secretion highlights the versatility and complexity of snoRNA-mediated regulation. Mechanistically, SNORA73 acts as molecular ternary glue, which the inventors termed ternary glue snoRNA (TAG-snoRNA), facilitating the association of mRNAs encoding secreted proteins with SRP, thereby promoting protein translocation and secretion. The 14-bp SNORA73-7SL duplex was detected in all five cell lines tested (FIG. 14C), suggesting that SNORA73 may play a general role in guiding protein translocation. Recent studies have shown that SNORA73 regulates hepatic metabolism and lipotoxicity in vivo⁸¹. The inventors speculate that the SNORA73-mediated mechanism of secretory and membrane protein translocation identified in HepG2 cells may be linked to its physiological roles in the liver. Moreover, snoRNA-mediated regulation of secretory and membrane proteins could have broader physiological implications in other tissues or cellular contexts, warranting future exploration.

The inventors identified additional snoRNA-7SL interactions (FIG. 14D), suggesting the presence of other TAG-snoRNAs involved in regulating protein translocation. For example, snoKARR-seq revealed interactions between SNORA44 and FGFR4 mRNA (encoding a secretory protein) as well as between SNORA44 and 7SL (FIG. 14E-F). KD of SNORA44 reduced FGFR4 secretion (FIG. 14G-H), representing another example of a TAG-snoRNA.

These TAG-snoRNAs may facilitate protein translocation in different cell types with different expression patterns. Their transcription could also be regulated in response to cellular signaling or environmental cues to add an additional layer of regulation on secretory and membrane proteins.

Engineering mRNA-snoRNA-7SL Interactions to Modulate Protein Secretion

The identification of the mRNA binding motif of SNORA73 allows TAG-snoRNA engineering to promote protein secretion via base-pairing rules. The principles of snoRNA-mediated rRNA modification have been applied to engineer snoRNAs for targeted mRNA modification in therapeutic applications⁸²⁻⁸⁴. Similarly, the base-pairing rules governing TAG-snoRNA interactions with secretory protein mRNAs could be leveraged to enhance the secretion of secretory proteins. With growing interest in optimizing the secretory pathway to increase biotherapeutic protein production⁸⁵⁻⁸⁷, mechanism-based snoRNA engineering offers a systematic and potentially more effective approach than the predominantly empirical tests currently used⁸⁸⁻⁹⁰.

In summary, the inventors report here snoKARR-seq for highly sensitive detection of snoRNA targets. The results suggest prevalent snoRNA-RNA interactions beyond rRNA and the presence of non-canonical roles of snoRNAs beyond RNA modification. The inventors discovered a non-canonical function of SNORA73 as a TAG-snoRNA in promoting protein translocation and secretion, representing a previously unknown RNA function. The application of snoKARR-seq offers opportunities for further snoRNA research, with the potential to uncover additional non-canonical functions of snoRNAs.

### Example 2: Materials and Methods for Certain Aspects Disclosed Herein Experimental Model and Study Participant Details

Cell culture HEK293T, HepG2, PC3, A549 and MDA-MB-231

HEK293T (ATCC, CRL11268), HepG2 (ATCC, HB8065), and MDA-MB-231 (ATCC, HTB-26) cells were cultured in DMEM (Gibco 11995), while PC3 (ATCC, CRL-1435) and A549 (ATCC, CCL-185) cells were cultured in DMEM/F-12 (Gibco 11320033). Both media were supplemented with 10% (v/v) fetal bovine serum (Gibco, 26140079), and 1% penicillin-streptomycin (Gibco, 15140122). All cells were maintained at 37 °C with 5% CO₂. To ensure the absence of mycoplasma contamination, all cell lines used in this study were examined using the LookOut Mycoplasma PCR Kit (Sigma-Aldrich, MP0035).

### Synthesizing DBCO and Biotin-Modified Dendrimers

1.53 pmol of PAMAM dendrimer G1 (Sigma-Aldrich, 412384), 3.06 μmol DBCO-NHS (Sigma-Aldrich, 761524), 1.53 μmol biotin-NHS (Sigma-Aldrich, 203112), and 5 μL triethylamine (Sigma-Aldrich, 471283) were dissolved in 2 mL methanol. The resulting solution was stirred overnight at room temperature. Following overnight stirring, 100 μL of acetic anhydride (Sigma, 320102) and 100 μL of triethylamine (Sigma-Aldrich, 471283) were added to modify any unreacted amine branches. The reaction mixture was stirred for an additional 24 hours at room temperature. To neutralize the reaction, 2 mL of water was added to the mixture. The dendrimer solution was then purified and concentrated using Microsep Advance Centrifugal Devices with Omega Membrane 1 K (Pall Corporation, MCP001C41) through series centrifugation at 5,000 g at 4 °C.

The characterization and quantification of modified dendrimers were conducted by measuring the characteristic UV absorbance of the DBCO moiety at 295 nm. To establish a calibration curve, a series of DBCO-NHS solutions with known concentrations were prepared as standard solutions. The UV absorbance at 295 nm of each standard solution was measured using a nanodrop spectrophotometer. The concentration of the dendrimer was determined by extrapolating its absorbance values onto the calibration curve.

The Design of ASO Targeting snoRNA cDNA

The top 50, 100, or 190 highly expressed snoRNAs, identified through small RNA-seq data from five different cell lines (FIG. 8A), were selected for ASO enrichment. snoRNA sequences obtained from snoDB³ were converted into cDNA. Primer3⁹⁵ was then used to design ASO probes against the snoRNA cDNA sequences. In cases where optimal ASO sequences could not be designed using Primer3⁹⁵, sequences that base pair with the middle 40 bases of the snoRNA cDNA sequences were employed as ASO sequences. 5′ biotinylated ASOs were purchased from Integrated DNA Technologies (IDT).

### SnoKARR-seq

Cells (at ~80% confluency) were crosslinked in 1% formaldehyde in culture medium for 10 min, followed by quenching with 25 mM glycine for 5 minutes. 6 million cells were resuspended in 500 μL of permeabilization buffer (10 mM Tris-HCl pH 8.0, 10 mM NaCl, 0.2% IGEPAL CA-630, 5 mM EDTA) supplemented with 5 mM N₃-kethoxal (made in-house³⁴), proteinase inhibitor (Roche, 11836170001), and SUPERNase inhibitor (Thermo Fisher, AM2696). Cells were rotated at room temperature for 30 minutes and subsequently collected by centrifugation at 2,500 g for 5 minutes. The cell pellet was washed by resuspending into 500 μL permeabilization buffer and collected by centrifugation. Next, the cells were resuspended in 500 μL of permeabilization buffer supplemented with 10 μM G1-DBCO-biotin dendrimer and incubated in a thermomixer at 1,000 rpm, 37 °C for 1 hour. Following the reaction, cells were collected and washed once as described above. Cell pellets were resuspended in 410 μL of 25 mM K₃BO₃, 50 μL of 10% SDS (Thermo Fisher, 15553027), 30 μL proteinase K (Thermo Fisher, 25530049), and 10 μL SUPERNase inhibitor (Thermo Fisher, AM2696). The mixture was incubated at 55 °C for 2 hours and subjected to phenol-chloroform (Thermo Fisher, AM9722) extraction and ethanol precipitation.

The precipitated RNA was dissolved in 104 μL of 25 mM K₃BO₃, 12 μL of 10× DNase I buffer (Thermo Fisher, AM8170G), 2 μL DNase I (Thermo Fisher, 18047019), and 2 μL SUPERNase inhibitor. The mixture was gently shaken at 37 °C for 30 min. Subsequently, 130 μL of 2×proteinase K buffer (100 mM Tris-HCl pH 7.5, 200 mM NaCl, 2 mM EDTA, 1% SDS) and 10 μL of proteinase K (Thermo, 25530049) were added. The resulting mixture was shaken at 1,000 rpm at 55 °C for another 30 minutes, followed by phenol-chloroform extraction and ethanol precipitation. The precipitated RNA was then dissolved in a mixture composed of 61 μL of 25 mM K₃BO₃, 7 μL of 10× RNA fragmentation buffer (Thermo Fisher, AM8740), and 2 μL of SUPERNase inhibitor. This mixture was heated at 70 °C for 15 minutes, and the reaction was quenched by adding 8 μL of fragmentation stop buffer (Thermo Fisher, AM8740).

For each sample, 30 μL of Dynabeads MyOne Streptavidin C1 (Thermo Fisher, 65001) was prepared by washing once with 100 μL of 1× binding/wash buffer (5 mM Tris-HCl pH 7.4, 0.5 mM EDTA, 1 M NaCl, 0.05% Tween-20), once with 100 μL of buffer A (100 mM NaOH, 50 mM NaCl), and once with 100 μL of buffer B (100 mM NaCl). Next, the beads were blocked by incubating with 100 μL of binding/wash buffer containing 1 μg/μL BSA (NEB, B9000S) and 1 μg/μL salmon sperm DNA (Thermo Fisher, 15632011) while rotating at room temperature for 30 minutes. The beads were then washed once with 100 μL of 1× binding and wash buffer, followed by resuspension in 80 μL of 2× binding/wash buffer (10 mM Tris-HCl pH 7.4, 1 mM EDTA, 2 M NaCl, 0.1% Tween-20) and mixed with the fragmented RNA. The beads-RNA mixture was incubated at room temperature for 20 minutes with gentle rotation, followed by washing twice with 100 μL of 1× binding/wash buffer and once with 100 μL of 1× PNK buffer (diluted from 10× PNK buffer from NEB, M0201L).

The beads were resuspended in 41 μL of 25 mM K₃BO₃. Add 5 μL of 10× T4 PNK buffer, 3 μL of T4 PNK (NEB, M0201L), and 1 μL of SUPERNase inhibitor. The beads were shaken at 1000 rpm at 37 °C for 30 minutes. Then an additional 1 μL of 10× T4 PNK buffer, 3 μL of T4 PNK (NEB, M0201L), and 6 μL of 10 mM ATP (NEB, P0756S) were added to the reaction mixture. The tube was shaken at 37 °C for another 30 minutes. After the reaction, the beads were washed twice with 100 μL of 1× binding/wash buffer and once with 100 μL of 1× ligation buffer (diluted from 10× T4 RNA ligase buffer from NEB, M0437M). For the ligation reaction, the beads were resuspended in 660 μL of 25 mM K₃BO₃, 100 μL of 10× T4 RNA ligase buffer, 2 μL of 10 mM ATP, 200 μL of 50% PEG 8000, 20 μL of T4 RNA ligase I (NEB, M0437M), and 10 μL of SUPERNase inhibitor. The reaction mixture was shaken at 1000 rpm at 16 °C for 16 hours.

After ligation, the beads were collected and washed three times with 1× binding/wash buffer. RNA was eluted by heating the beads in 50 μL of H₂O at 95 °C for 10 minutes. The RNA was purified using the RNA Clean & Concentrator kit (Zymo, R1014) following the manufacturer's protocol and eluted with 30 μL of H₂O. The RNA concentration was measured, and 10-50 ng of RNA was used for reverse transcription (RT) using the RT reagents in the SMARTer Stranded Total RNA-seq kit v2-pico input mammalian (Takara, 634413). After RT, 2 μL of RNase H (NEB M0297S) was added to each sample, which was then incubated for 20 minutes at 37 °C. The reaction was quenched by heating inactivation for 5 minutes at 70 °C. Subsequently, cDNA was purified using the DNA Clean & Concentrator-5 kit. 10% of the cDNA was saved as an input sample, while the remaining cDNA was used for snoRNA enrichment.

30 μL of cDNA was combined with 5 μL biotinylated ASO mix (1 μM, purchased from IDT) and 15 μL hybridization buffer (250 mM HEPES, pH 7.0, 500 mM KCl). The hybridization reaction was run in a thermocycler using the following conditions: 95 °C for 7 minutes, then 95 °C for 10 seconds (ramp rate 0.1 °C/s, increment −0.1 °C/cycle, 750 cycles), followed by cooling to 4 °C indefinitely. 30 μL of Dynabeads MyOne Streptavidin C1 (Thermo Fisher, 65001) were prepared by washing once with 100 μL of 1× binding and wash buffer (5 mM Tris-HCl pH 7.4, 0.5 mM EDTA, 1 M NaCl), once with 100 μL of buffer A (100 mM NaOH, 50 mM NaCl), once with 100 μL of buffer B (100 mM NaCl), and once with 1× binding and wash buffer again. The beads were then resuspended in 50 μL of 2× binding and wash buffer (10 mM Tris-HCl pH 7.4, 1 mM EDTA, 2 M NaCl). Next, the beads were mixed with the hybridization reaction mixtures and incubated at 4 °C for 30 minutes. The beads were then washed four times with 100 μL of 1× binding and wash buffer, followed by one quick wash with 100 μL of H₂O. The cDNA was eluted by heating the beads in 20 μL of H₂O at 75 °C for 5 minutes. Both the input and eluted cDNA samples were subject to library construction following the SMARTer Stranded Total RNA-seq kit v2 (Takara, 634413) protocol. During the library construction, the ZapR v2 treatment for ribosomal cDNA cleavage was skipped when preparing samples for detecting snoRNA-rRNA interactions and was included (rRNA depleted in FIG. 9D) when preparing samples for detecting snoRNA-mRNA interactions. The libraries were sequenced on the Illumina NovaSeq4000 platform in PE150 mode, with approximately 80 million reads per sample.

For snoKARR-seq performed using mouse brain tissue, the tissue was homogenized to obtain a cell suspension using a Dounce homogenizer in ice-cold PBS. The cell suspension was then centrifuged at 100 g for 15 seconds to sediment and separate potential large tissue pieces at the bottom of the tube. The supernatant was transferred to a clean microcentrifuge tube. Subsequently, the supernatant was centrifuged at 800 g for 5 minutes. The supernatant was removed, and the cell pellet at the bottom of the tube was used for the typical snoKARR-seq procedures as described above.

### SnoKARR-Seq Data Processing, Analysis, and Visualization

Adaptors from raw reads were trimmed, and the overlapping read pairs (2×150 bp) were merged into single-end reads and de-duplicated using fastp⁹⁶. The resulting single-end FASTQ files were then aligned to a customized reference genome containing human rDNA sequence (GenBank. KY962518.1) and snoRNA sequences (snoRNA annotation was acquired from snoDB³) using STAR⁹⁸ to map snoRNA-rRNA interactions. The following STAR parameters were used to recover gapped and chimeric reads based on a prior study¹⁰⁶: --runMode alignReads --outFilterMultimapNmax 20--chimMultimapNmax 10000--alignIntronMin 1--scoreGap 0--scoreGapNoncan 0--scoreGapGCAG 0--scoreGapATAC 0--scoreGenomicLengthLog2scale -1--chimFilter None --chimOutType WithinBAM HardClip --chimSegmentMin 5--chimJunctionOverhangMin 5--chimScoreJunctionNonGTAG 0--chimScoreDropMax 80--chimNonchimScoreDropMin 20--peOverlapNbasesMin 12--peOverlapMMp 0.05--limitOutSJcollapsed 10000000). The known snoRNA-rRNA interactions that lead to RNA modifications were obtained from snoRNA-LBME-db¹, snOPY², snoDB³, and snoRNA Atlas⁴. The box sequences of snoRNAs were obtained from snoRNA Atlas⁴.

To map interactions between snoRNA and non-rRNA, FASTQ files were aligned to the human rDNA sequence (GenBank. KY962518.1). The unmapped reads were then used to map to the hg38 human genome using STAR. Chimeric reads were extracted from the resulting BAM file and reads containing snoRNAs were filtered out based on annotations from snoDB 3. The coordinate pairs (snoRNA and the target) were then subjected to duplex group (DG) clustering using a spectral clustering algorithm¹⁰⁶ For data from cell lines, clusters containing at least two chimeric reads from both replicates were considered as reproducible interactions. For snord115/116 targets in mouse brain samples, clusters containing at least one chimeric read from both replicates were considered as reproducible interactions, considering the low abundance of snord115/116 in mouse brain samples. The overlapping regions of the cluster from two replicates were used as the final coordinates of the snoRNA and the corresponding target. The snoRNA targets were annotated using annotatepeaks.pl in the HOMER suite⁶⁰. Published RIC-seq⁴⁷ and PARIS^{50,54} data were processed using the same pipeline.

The quantification of snoRNA reads in both the KARR-seq (unenriched) and snoKARR-seq (enriched) was performed using featureCounts¹⁰⁷ and DESeq2¹⁰⁸. The structure and free energy of the duplex formed between snoRNA and their targets identified were predicted using RNAplex (with option “-c 40, -1 40”)¹⁰⁴ and visualized using VARNA¹⁰⁹ To visualize the binding regions of snoRNA, the bed file containing snoRNA binding regions was converted to a BigWig file using bedGraphToBigWig¹¹⁰. Genome tracks displaying snoRNA target coverage were plotted using CoolBox¹¹¹. To visualize 2D snoRNA-rRNA interactions (FIG. 2C-D), the coverage of snoRNA/rRNA to 10 nt-sized bins in snoRNA and rRNA was calculated and plotted in a heatmap. The meta distribution of binding sites of SNORA73 on mRNA (FIG. 4E) was generated by the R package Guitar¹¹². The sequence motif analysis of SNORA73 mRNA targets was performed using findMotifs.pl in the HOMER suite⁶⁰, with random background sequences generated by the bedtools shuffle in the BEDTools suite¹⁰⁰. GO analysis was performed using the GSEApy module¹¹³ The secretome genes were obtained from SPRomeDB¹¹⁴, the human protein atlas¹¹⁵ and the genes in the GO term extracellular space (0005615). The membranome genes were obtained from the Membranome¹¹⁶ and the genes in the Go term membrane 0016020. To assess the reproducibility of snoKARR-seq, multiBamSummary from deepTools¹⁰⁵ was used to calculate the genomic coverage correlation, and unique chimeric read counts from each DG group in two replicates were used to evaluate the correlation of chimeric reads. Unique chimeric read counts for each DG group were obtained by intersecting the snoRNA and target coordinates in each DG group with raw chimeric read coordinates. For homology analysis, conserved snoRNA sequences were obtained from Rfam¹¹⁷, and mRNA sequences were obtained through UCSC LiftOver¹⁰³ To calculate the signal-to-noise ratio for known snoRNA-rRNA interactions, the known modification sites +/−100 nt regions were defined as signals, while the upstream and downstream 300~500 nt regions were defined as noise.

### Differentiate Between Proximal Tertiary Interactions and Direct Base-Pairing Interactions.

snoKARR-seq captures RNAs in proximity, the varying signals observed in the unprocessed, raw snoKARR-seq profiles of snoRNA-rRNA interactions (e.g. FIG. 9A-D) likely result from proximity effects due to the complex 3D architecture of rRNA and snoRNAs. For instance, while a snoRNA may base pair with a specific region of rRNA, other parts of the snoRNA may be in close proximity to different rRNA regions in the 3D structure, generating signals in the snoKARR-seq profiles.

The inventors employed two strategies to differentiate between proximal and base-pairing snoRNA-rRNA interactions:

De Novo Duplex Identification (DDI): The inventors performed duplex structure prediction for each snoRNA-rRNA DG group to identify de novo duplexes. Interactions with free energy lower than −17 kcal/mol (the highest free energy of known snoRNA-rRNA duplexes) were categorized as base-pairing interactions, while the others were classified as proximal interactions.

Proximal interaction Removal (PIR): To further eliminate potential proximal interactions, the inventors conducted structural modeling of snoRNA-rRNA interactions. The inventors calculated the interaction radius of snoRNAs (the longest distance between any two residues within snoRNA structure models generated by AlphaFold 3¹¹⁸). The inventors then extracted rRNA residue coordinates from a human 80S ribosome cryoEM structure (PDBID: 4UGO¹¹⁹) and assumed that any rRNA residues within this radius (on average ~106 Å, which is larger than the size of the dendrimer 22 Å) of the known rRNA binding site of a given snoRNA are in proximity to the known interaction. These interactions were classified as proximal interactions.

For snoRNAs with known rRNA binding targets (41 out of 50 enriched), the inventors used a combination of DDI and PIR methods (FIG. 9A-B). For snoRNAs without known rRNA targets (9 out of 50 enriched), the inventors applied the DDI method alone (FIG. 9C).

When analyzing snoRNA targets other than rRNA, the inventors used the DDI strategy to distinguish between proximal and base-pairing interactions (at least 8 bps). The inventors set the threshold as 8 bps because N₃-kethoxal labeling does not disrupt duplex structures with as few as 8 bps under the treatment conditions used in snoKARR-seq (FIG. 14I). To ensure that the inventors capture only stable duplex structures, the inventors used RNAplex¹⁰⁴ to predict RNA duplex structures, setting the maximum duplex length to 40 bps and penalizing consecutive mismatches (parameters 1=40 and c=40 in RNAplex¹⁰⁴) The resulting RNA duplex length is on average ~12 bps (FIG. 14J).

To differentiate between direct base pairing and indirect proximity effect or tertiary interactions for snoRNA-non rRNA interactions, the inventors set a threshold of 8 bps, categorizing predicted structures with fewer bps as potential proximal/tertiary interactions rather than direct base-pairing. For non-rRNA targets of snoRNAs, ~69-79% are categorized as potential duplex interactions across different cell lines (FIG. 14K).

### Small RNA-seq

Small RNA fractions were isolated using mirVana™ miRNA Isolation Kit (Invitrogen, AM1561) according to the manufacturer's protocol. The resulting small RNA fraction was subsequently used for sequencing library construction using the NEB Next Small RNA Library Prep Set for Illumina (NEB E7330S) with the Nₘ-Mut-seq protocol adapted from a prior study¹⁹. The libraries were sequenced on the Illumina NovaSeq4000 platform in SE100 mode, with approximately 30 million reads per sample.

Adaptors and low-quality reads were removed from raw reads using cutadapt 97 Duplications were removed using Clumpify (BBMap tool v.38.73). 5-mer random barcodes at read ends were trimmed, and low-quality or short reads (less than 20 nt) were removed using cutadapt 97. The remaining reads were aligned to the hg38 genome using HISAT2¹⁰¹ and the expression of snoRNAs was quantified using StringTie¹⁰².

### Isolation of Cytosolic Ribosome, Cytosolic, and Nuclear Fractions.

HepG2 cells were lysed in 500 μL of lysis buffer containing 200 mM HEPES pH 7.6, 1M KCl, 10 mM MgCl2, 250 mM sucrose, and 10% Triton-X100, supplemented with proteinase inhibitor and SUPERNase inhibitor. Following a 20-minute incubation on ice with periodic perturbation, the lysate was centrifuged at 18,000 g for 15 minutes at 4 °C, resulting in the separation of nuclei in the pellet and the cytosolic components in the supernatant. Subsequently, 200 μL of the supernatant was carefully transferred to 300 μL of sucrose cushion (200 mM HEPES pH 7.6, 1M KCl, 10 mM MgCl₂, 500 mM sucrose, 10% Triton-X100) in a 0.5 mL, open-top thickwall polycarbonate tube (Beckman Coulter, 343776). The sample was then subjected to ultracentrifugation for 1 hour at 355,000 g at 4 °C. Following ultracentrifugation, the supernatant contained the ribosome-free cytosolic fraction, while the pellet contained the cytosolic ribosomes. The pellet was either suspended in lysis buffer for western blot or subjected to RNA extraction using TRIzol (Invitrogen, 15596026). To directly isolate cytosolic and nuclear RNA, the Cytoplasmic and Nuclear RNA Purification Kit from (Norgen Biotek, 21000) was used.

### Microsome Isolation

HepG2 cells (~80% confluency) from a 10 cm plate were treated with either DMSO or MG-132 (final concentration 10 μM for 4 hours), harvested, and resuspended in 1 mL of homogenization buffer (10 mM HEPES-KOH pH 7.5, 10 mM KOAc, 1 mM MgCl₂) supplemented with proteinase inhibitor and SUPERNase inhibitor. The mixture was then incubated on ice for 20 minutes with periodic perturbation, followed by centrifugation at 18,000 g for 15 minutes at 4 °C. Subsequently, 40 strokes of the tissue grinder (DWK Life Sciences, 985-44233-E1) were used to homogenize the cells. Sucrose was added to the lysate to a final concentration of 250 mM, followed by centrifugation for 15 minutes at 750 g, 4 °C. The supernatant was saved, and the centrifugation was repeated to completely remove the nuclei. The supernatant was again saved, and centrifugation was performed for 15 minutes at 19,357 g, 4 °C. The resulting supernatant constituted the cytosolic fraction, while the pellet contained the microsomes, including the ER.

Preparation of Secreted Protein Samples from Cell Culture Media

Cell culture media was collected and centrifuged at 1500 rpm for 10 minutes at 4 °C to remove debris. Subsequently, four times the sample volume of cold (−20 °C) acetone was added to the media, and the mixture was incubated at −20 °C for at least two hours to precipitate proteins. Following incubation, the mixture was centrifuged for 10 minutes at 15,000 g to pellet the precipitated proteins, and the supernatant was discarded. The protein pellet was then air-dried and resuspended in CelLytic™ M lysis buffer (Sigma-Aldrich, C2978) for western blot.

### Western Blot

Proteins extracted from various cellular fractions, including whole cell lysates, cell culture media, cytosolic fractions, ribosome-free cytosolic fractions, cytosolic ribosome fractions, nuclei, and ER fractions, were separated by SDS-PAGE and transferred onto nitrocellulose membranes (Bio-Rad). Following the transfer, the membranes were blocked with 5% milk in PBST (0.1% Tween-20) and then incubated overnight with primary antibodies diluted in 1% milk in PBST at 4 °C. The next day, the membranes were washed five times with PBST and then incubated with secondary antibodies for 1 hour at room temperature. After incubation with secondary antibodies, the membranes were washed three times with PBST before being subjected to enhanced chemiluminescence (ECL) incubation and developed for imaging.

ASO LNA siRNA Knockdown and 2′-MOE Steric Block

The ASO, LNA, and 2′-MOE-modified sequences used to knock down/block snoRNAs or mRNA were purchased from IDT. Transfection was performed using Lipofectamine™ RNAiMAX (Invitrogen 13778150) following the reverse transfection protocol. The final concentration of both ASO and LNA was adjusted to 80 nM. The final concentration for 2′-MOE-modified sequences is 150 nM. The MBM and 7BM steric blocks are a 1:1 mixture of sequences targeting MBM/7BM in SNORA73A and SNORA73B. Following transfection, cells were cultured for 2-3 days before harvest for downstream analyses. siRNAs transfection was performed using Lipofectamine™ RNAiMAX (Invitrogen 13778150) following the reverse transfection protocol. 2 days post-transfection, the media was removed, and a second round of forward transfection was performed to reach optimal KD efficiency. After a second round of incubation of 1-2 days, the cell culture media and cell lysate were collected. The cell culture media was collected for ELISA analysis following the manufacturer's instructions for the respective ELISA kits. The secreted proteins recovered from the media and intracellular proteins and RNAs recovered from total cell lysate and different cellular fractions were subjected to western blot analysis or RT-qPCR analysis respectively.

### RT-qPCR

Total RNA or RNA from different cellular fractions was extracted using TRIzol reagent (Invitrogen, 15596026) according to the manufacturer's instructions. Subsequently, the extracted RNA was reverse transcribed using PrimeScript RT Master Mix (Takara, RR0361) with both oligo dT primer and random 6-mers primer to ensure efficient reverse transcription of both polyadenylated and non-polyadenylated RNA species. The resulting cDNA was then subjected to real-time PCR analysis using the LightCycler 96 system (Roche) and FastStart Essential DNA Green Master (Roche, 06402712001) with gene-specific primers. Relative changes in gene expression were calculated using the ΔΔCt method.

To validate snoRNA-rRNA interactions (FIG. 2E), crosslinked RNA samples from KARR-seq procedures were split into two halves. One half was subjected to overnight ligation and the other half was incubated overnight at the same condition but without the addition of the ligase. The RNA samples from both samples were then recovered using the RNA Clean & Concentrator kit (Zymo, R1014). A synthetic spike-in DNA sequence (the Cypridina Luciferase DNA sequence from the EpiMark® N6-Methyladenosine Enrichment Kit, NEB, E1610S) was added and used as the reference for RNA quantification. Primer pairs designed to target snoRNA and the corresponding rRNA targets were used to detect the formation of chimeric reads encoding such snoRNA-rRNA interactions All primer sequences were purchased from IDT.

snoRNA Expression

snoRNAs were expressed under the hU6 promoter as designed based on a prior study⁸². The snoRNA DNA sequences were purchased as gBlocks from IDT and subsequently cloned into the guide snoRNA plasmid using NEBuilder® HiFi DNA Assembly Master Mix (NEB, E2621S) according to the manufacturer's instructions. All clones were verified by Sanger sequencing conducted through the University of Chicago Comprehensive Cancer Center DNA Sequencing and Genotyping Facility. Transfection was carried out using Lipofectamine™ 3000 Transfection Reagent (Invitrogen, L3000015) according to the manufacturer's instructions. The successful expression of snoRNA was assessed by RT-qPCR. For the rescue experiments (FIG. 5D, 5H, 12N), HepG2 cells were treated with LNA for 48 hours followed by transfection of different snoRNA constructs in fresh media. Cells or culture media were collected after 48 hours of transfection.

mCherry and eGFP Reporter Assay

The CLU-mCherry, secreted eGFP (seGFP), and secreted mCherry (smCherry) constructs were purchased from VectorBuilder using the mammalian gene expression vector with a CMV promoter. In the CLU-mCherry construct, the mCherry sequence was fused to the C-terminal of the CLU mRNA sequence. Additionally, the N-terminal signal sequence of a GPI-anchored T-cadherin was fused to the N terminus of eGFP or mCherry 74.

HepG2 cells were cultured in phenol-free media (Gibco, 21063029) to avoid autofluorescence interference from phenol red. Cells were grown overnight in a 24-well plate until reaching approximately 75-90% confluence. Transfections were performed with CLU-mCherry or seGFP along with snoRNA constructs, with a total DNA amount of 500 ng and a molar ratio of CLU-mCherry/seGFP:snoRNA of 1:3, using Lipofectamine™ 3000 Transfection Reagent (Invitrogen, L3000015) in phenol-free Opti-MEM™ (Gibco, 11058021). As a transfection control, when transfecting cells with CLU-mCherry and snoRNAs, an equivalent amount of seGFP as CLU-mCherry was co-transfected. Similarly, when transfecting cells with seGFP and snoRNA, smCherry was co-transfected. 48 hours post-transfection, cell culture medium was collected, centrifuged at 15,000 rpm for 1 min, and 100 L was added to a black 96-well plate in triplicate. The eGFP signal was measured using an excitation and emission wavelength/bandwidth of 485/20 nm and 545/20 nm, respectively, while the mCherry signal was measured using an excitation and emission wavelength/bandwidth of 587/20 nm and 645/20 nm, respectively, using a Synergy Neo2 multi-mode reader (BioTek). The GFP/mCherry signal was normalized to their corresponding mCherry/GFP transfection control signal.

### SRP RIP-qPCR

HepG2 cell pellets from a 10-cm plate (~80% confluency) were lysed with 3 volumes of lysis buffer (150 mM KCl, 10 mM HEPES pH 7.6, 2 mM EDTA, 0.5% NP-40, 0.5 mM DTT) supplemented with proteinase inhibitor and SUPERNase inhibitor on ice for 20 minutes. The lysates were then centrifuged at 16,000 g for 15 minutes, with 10% of the supernatant saved as input and mixed with 1 mL TRIzol for RNA extraction. To the remaining lysate, either 5 g of rabbit IgG (Abcam ab172730) or SRP14/72 antibodies were added and incubated by rotating overnight at 4 °C. Following incubation, 40 μL of Pierce™ Protein A/G Magnetic Beads (Thermo Fisher, 88802) was washed three times with lysis buffer and added to the lysate, followed by a 3-hour incubation by rotation at 4 °C. The beads were then washed five times with 500 μL wash buffer (200 mM NaCl, 2 mM EDTA, 0.05% NP-40, 50 mM Tris-HCl pH 7.5, 0.5 mM DTT, supplemented with protease inhibitor and RNase inhibitor) and resuspended in 90 μL wash buffer. 10 μL were saved for western blot analysis, while the remaining 80 μL was combined with 100 μL 2× protease K buffer (100 mM Tris-HCl pH 7.6, 150 mM NaCl, 12.5 mM EDTA, 2% w/v SDS) and 20 μL protease K (Thermo Scientific, E00491). The mixture was gently shaken at 55 °C for 40 minutes. After separation using a magnetic rack, RNA from the eluent was extracted using TRIzol reagent followed by RT-qPCR analysis. The Ct value of each RIP RNA fraction was normalized to the Input RNA fraction Ct value. This normalized RIP fraction Ct value was further normalized against the normalized background (IgG) fraction Ct to generate the ΔΔCt value. In the double-pulldown experiment (FIG. 13J), a second round of ASO pulldown was performed. After the proteinase K treatment, RNA was purified using phenol-chloroform extraction and ethanol precipitation. ASOs designed to enrich CLU mRNA sequences, or a control ASO targeting GFP sequences, were incubated with RNA samples overnight at 37 °C in the hybridization buffer (250 mM HEPES, pH 7.0, 500 mM KCl). ASO-enriched RNAs were pulled down using Dynabeads MyOne Streptavidin C1 beads, following the manufacturer's protocol.

### Quantification of Nm by LC-MS/MS

Total RNA extracted from control and U3/U8 KD HepG2 cells underwent stringent purification steps to enrich for mRNA and remove ribosomal RNA. This was achieved through two rounds of mRNA purification using the Dynabeads™ mRNA DIRECT™ Purification Kit (Invitrogen, 61012) followed by two rounds of ribosomal RNA depletion using the RiboMinus™ Eukaryote System v2 (Invitrogen, A15026). The resulting RNA, approximately 200 ng, was then subjected to enzymatic digestion by nuclease P1 (1 U Sigma-Aldrich, N8630) in a buffer containing 25 mM NaCl and 2.5 mM ZnCl₂ for 1 hour at 42 °C. Subsequently, 1 unit of FastAP (Thermo Scientific, EF0654) was added in 10× FastAP buffer, and the mixture was incubated at 37 °C for 4 hours. The digested sample was filtered using a 0.22 μm pore size membrane (Millipore, GSWPO4700), and 5 μL of the resulting solution was injected into a C18 reverse-phase column coupled to an Agilent 6460 LC-MS/MS spectrometer operating in positive electrospray ionization mode. Quantification of 2′-O-methylation-containing nucleosides was performed based on their retention time and the nucleoside-to-base ion mass transitions: 268-136 (A), 282-136 (Am), 244-112 (C), 258-112 (Cm), 284-152 (G), and 298-152 (Gm). Nucleoside levels were quantified by comparison with a standard curve generated from pure nucleoside standards analyzed under identical conditions.

### Polysome Profiling

HepG2 cells were treated with 100 μg/ml cycloheximide (Abcam, ab120093) for 8 minutes, followed by harvesting and resuspension in 500 μl lysis buffer consisting of 5 mM Tris-HCl pH 7.5, 15 mM KCl, 5 mM MgCl2, 0.5% Triton X-100, 0.5% sodium deoxycholate, 1 mM DTT, 100 g/mL cycloheximide, and 10 U/mL Turbo DNase, supplemented with proteinase inhibitor, and SUPERNase inhibitor. The cleared cell lysate was obtained by centrifugation at 15,000 g for 10 minutes at 4 °C. Subsequently, the lysate was subjected to density gradient ultracentrifugation at 28,000 rpm at 4 °C for 3 hours on 10-50% linear sucrose gradients in buffer containing 20 mM Tris-HCl pH 7.5, 150 mM NaCl, 5 mM MgCl2, 1 mM DTT, and 100 μg/ml cycloheximide. The gradients were fractionated into 0.6 mL fractions, which were then analyzed by both RT-qPCR and western blot.

### In Vitro Transcription and RNA-RNA Interaction Analysis

SNORA73B and SNORA15A were in vitro transcribed from gBlocks (ordered from IDT) using T7 RNA polymerase (NEB E2040) following the manufacturer's protocol. The respective amount of nuclease-free water was added to dilute the RNA to approximately 37.5 fmol/μL. Fluorescein-labeled CLUmRNA and 7SL RNA (ordered from IDT) were diluted with nuclease-free water to approximately 75 fmol/μL.

The hybridization reaction consisted of 6 μL of hybridization buffer (250 mM HEPES, pH 7.0, 500 mM KCl), 4 μL of each RNA, and nuclease-free water to a final reaction volume of 18 μL. For control conditions (CLU, 7SL, CLU+7SL), 300 fmol of each fluorescein-labeled RNA was added to the hybridization reaction. For snoRNAs, 150 fmol (4 μL) of each snoRNA and 300 fmol (4 μL) of each fluorescein-labeled RNA were added to the hybridization reaction. The final molar ratio between SNORA and total fluorescein-labeled RNA was 1:2 (for snoRNA-CLU or snoRNA-7SL) or 1:4 (for snoRNA-CLU-7SL). The hybridization reaction was conducted in a thermocycler using the ASO cDNA enrichment PCR program: 95 °C for 7 min, 95 °C for 10 s (ramp rate 0.1 °C/s, increment −0.1 °C/cycle, 750 cycles), 4 °C indefinitely. RNA loading dye (2×) (NEB B0363S) was added to each reaction mixture which was then loaded onto a 4-20% Novex TBE gel (ThermoFisher EC62255). The gel was run at 100 V for 1 hour at 4 °C and then imaged using the fluorescein channel (532/28 filter, blue epi-illumination) on a BioRad ChemiDoc MP imager (UChicago Biophysics Core).

### Quantification and Statistical Analysis

P values were determined by a paired two-tailed t-test indicated in the figure legends. *p<0.05, **p<0.01. Error bars represent mean±s.d. For box plots, the center line represents the median, the box limits show the upper and lower quartiles and whiskers represent 1.5× the interquartile range.

Examples also include those described in Liu et al. Cell. 2025 Jan. 23; 188(2):465-483.e22, which is hereby incorporated by reference in its entirety.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference in their entirety.
1. Lestrade, L., and Weber, M. J. (2006). snoRNA-LBME-db, a comprehensive database of human H/ACA and C/D box snoRNAs. Nucleic Acids Res 34, D158-162. 10.1093/nar/gkj002.
2. Yoshihama, M., Nakao, A., and Kenmochi, N. (2013). snOPY: a small nucleolar RNA orthological gene database. BMC Res Notes 6, 426. 10.1186/1756-0500-6-426.
3. Bouchard-Bourelle, P., Desjardins-Henri, C., Mathurin-St-Pierre, D., Deschamps-Francoeur, G., Fafard-Couture, E., Garant, J. M., Elela, S. A., and Scott, M. S. (2020). snoDB: an interactive database of human snoRNA sequences, abundance and interactions. Nucleic Acids Res 48, D220-D225. 10.1093/nar/gkz884.
4. Jorjani, H., Kehr, S., Jedlinski, D. J., Gumienny, R., Hertel, J., Stadler, P. F., Zavolan, M., and Gruber, A. R. (2016). An updated human snoRNAome. Nucleic Acids Res 44, 5068-5082. 10.1093/nar/gkw386.
5. Maxwell, E. S., and Fournier, M. J. (1995). The small nucleolar RNAs. Annu Rev Biochem 64, 897-934. 10.1146/annurev.bi.64.070195.004341.
6. Balakin, A. G., Smith, L., and Fournier, M. J. (1996). The RNA world of the nucleolus: two major families of small RNAs defined by different box elements with related functions. Cell 86, 823-834. 10.1016/s0092-8674(00)80156-7.
7. Tollervey, D., and Kiss, T. (1997). Function and synthesis of small nucleolar RNAs. Curr Opin Cell Biol 9, 337-342. 10.1016/s0955-0674(97)80005-1.
8. Darzacq, X., Jady, B. E., Verheggen, C., Kiss, A. M., Bertrand, E., and Kiss, T. (2002). Cajal body-specific small nuclear RNAs: a novel class of 2′-O-methylation and pseudouridylation guide RNAs. EMBO J 21, 2746-2756. 10.1093/emboj/21.11.2746.
9. Kiss, T., and Filipowicz, W. (1995). Exonucleolytic processing of small nucleolar RNAs from pre-mRNA introns. Genes Dev 9, 1411-1424. 10.1101/gad.9.11.1411.
10. Kufel, J., and Grzechnik, P. (2019). Small Nucleolar RNAs Tell a Different Tale. Trends Genet 35, 104-117. 10.1016/j.tig.2018.11.005.
11. Xing, Y. H., and Chen, L. L. (2018). Processing and roles of snoRNA-ended long noncoding RNAs. Crit Rev Biochem Mol Biol 53, 596-606. 10.1080/10409238.2018.1508411.
12. Kiss, T. (2002). Small nucleolar RNAs: an abundant group of noncoding RNAs with diverse cellular functions. Cell 109, 145-148. 10.1016/s0092-8674(02)00718-3.
13. Reichow, S. L., Hamma, T., Ferre-D′ Amare, A. R., and Varani, G. (2007). The structure and function of small nucleolar ribonucleoproteins. Nucleic Acids Res 35, 1452-1464. 10.1093/nar/gkl 1172.
14. Kiss-Laszlo, Z., Henry, Y., Bachellerie, J. P., Caizergues-Ferrer, M., and Kiss, T. (1996). Site-specific ribose methylation of preribosomal RNA: a novel function for small nucleolar RNAs. Cell 85, 1077-1088. 10.1016/s0092-8674(00)81308-2.
15. Karijolich, J., and Yu, Y. T. (2010). Spliceosomal snRNA modifications and their function. RNA Biol 7, 192-204. 10.4161/rna.7.2.11207.
16. Erales, J., Marchand, V., Panthu, B., Gillot, S., Belin, S., Ghayad, S. E., Garcia, M., Laforets, F., Marcel, V., Baudin-Baillieu, A., et al. (2017). Evidence for rRNA 2′-O-methylation plasticity: Control of intrinsic translational capabilities of human ribosomes. Proc Natl Acad Sci USA 114, 12934-12939. 10.1073/pnas.1707674114.
17. Charette, M., and Gray, M. W. (2000). Pseudouridine in RNA: what, where, how, and why. IUBMB Life 49, 341-351. 10.1080/152165400410182.
18. Ge, J., and Yu, Y. T. (2013). RNA pseudouridylation: new insights into an old modification. Trends Biochem Sci 38, 210-218. 10.1016/j.tibs.2013.01.002.
19. Chen, L., Zhang, L. S., Ye, C., Zhou, H., Liu, B., Gao, B., Deng, Z., Zhao, C., He, C., and Dickinson, B. C. (2023). Nₘ-Mut-seq: a base-resolution quantitative method for mapping transcriptome-wide 2′-O-methylation. Cell Res. 10.1038/s41422-023-00836-w.
20. Elliott, B. A., Ho, H. T., Ranganathan, S. V., Vangaveti, S., Ilkayeva, O., Abou Assi, H., Choi, A. K., Agris, P. F., and Holley, C. L. (2019). Modification of messenger RNA by 2′-O-methylation regulates gene expression in vivo. Nat Commun 10, 3401. 10.1038/s41467-019-11375-7.
21. Yin, Q. F., Yang, L., Zhang, Y., Xiang, J. F., Wu, Y. W., Carmichael, G. G., and Chen, L. L. (2012). Long noncoding RNAs with snoRNA ends. Mol Cell 48, 219-230. 10.1016/j.molcel.2012.07.033.
22. Vitali, P., and Kiss, T. (2019). Cooperative 2′-O-methylation of the wobble cytidine of human elongator tRNA(Met)(CAT) by a nucleolar and a Cajal body-specific box C/D RNP. Genes Dev 33, 741-746. 10.1101/gad.326363.119.
23. Zhang, M., Li, K., Bai, J., Damme, R. V., Zhang, W., Alba, M., Stiles, B. L., Chen, J.-F., and Lu, Z. (2023). snoRNA-guided tRNA 2′-O-methylation controls codon-biased gene expression and cellular states. bioRxiv, 2023.2006.2022.546166. 10.1101/2023.06.22.546166.
24. Cheng, Y., Wang, S., Zhang, H., Lee, J. S., Ni, C., Guo, J., Chen, E., Wang, S., Acharya, A., Chang, T. C., et al. (2024). A non-canonical role for a small nucleolar RNA in ribosome biogenesis and senescence. Cell 187, 4770-4789 e4723. 10.1016/j.cell.2024.06.019.
25. Zhang, Y., Zhao, Z., Huang, L. A., Liu, Y., Yao, J., Sun, C., Li, Y., Zhang, Z., Ye, Y., Yuan, F., et al. (2023). Molecular mechanisms of snoRNA-IL-15 crosstalk in adipocyte lipolysis and NK cell rejuvenation. Cell Metab 35, 1457-1473 e1413. 10.1016/j.cmet.2023.05.009.
26. Falaleeva, M., Pages, A., Matuszek, Z., Hidmi, S., Agranat-Tamir, L., Korotkov, K., Nevo, Y., Eyras, E., Sperling, R., and Stamm, S. (2016). Dual function of C/D box small nucleolar RNAs in rRNA modification and alternative pre-mRNA splicing. Proc Natl Acad Sci USA 113, E1625-1634. 10.1073/pnas.1519292113.
27. Huang, C., Shi, J., Guo, Y., Huang, W., Huang, S., Ming, S., Wu, X., Zhang, R., Ding, J., Zhao, W., et al. (2017). A snoRNA modulates mRNA 3′ end processing and regulates the expression of a subset of mRNAs. Nucleic Acids Res 45, 8647-8660. 10.1093/nar/gkx651.
28. Vitali, P., Basyuk, E., Le Meur, E., Bertrand, E., Muscatelli, F., Cavaille, J., and Huttenhofer, A. (2005). ADAR2-mediated editing of RNA substrates in the nucleolus is inhibited by C/D small nucleolar RNAs. J Cell Biol 169, 745-753. 10.1083/jcb.200411129.
29. Gong, J., Li, Y., Liu, C. J., Xiang, Y., Li, C., Ye, Y., Zhang, Z., Hawke, D. H., Park, P. K., Diao, L., et al. (2017). A Pan-cancer Analysis of the Expression and Clinical Relevance of Small Nucleolar RNAs in Human Cancer. Cell Rep 21, 1968-1981. 10.1016/j.celrep.2017.10.070.
30. Sahoo, T., del Gaudio, D., German, J. R., Shinawi, M., Peters, S. U., Person, R. E., Garnica, A., Cheung, S. W., and Beaudet, A. L. (2008). Prader-Willi phenotype caused by paternal deficiency for the HBII-85 C/D box small nucleolar RNA cluster. Nat Genet 40, 719-721. 10.1038/ng.158.
31. Hebras, J., Marty, V., Personnaz, J., Mercier, P., Krogh, N., Nielsen, H., Aguirrebengoa, M., Seitz, H., Pradere, J. P., Guiard, B. P., and Cavaille, J. (2020). Reassessment of the involvement of Snord1 15 in the serotonin 2c receptor pathway in a genetically relevant mouse model. Elife 9. 10.7554/eLife.60862.
32. Jenkinson, E. M., Rodero, M. P., Kasher, P. R., Uggenti, C., Oojageer, A., Goosey, L. C., Rose, Y., Kershaw, C. J., Urquhart, J. E., Williams, S. G., et al. (2016). Mutations in SNORD118 cause the cerebral microangiopathy leukoencephalopathy with calcifications and cysts. Nat Genet 48, 1185-1192. 10.1038/ng.3661.
33. Wu, T., Cheng, A. Y., Zhang, Y., Xu, J., Wu, J., Wen, L., Li, X., Liu, B., Dou, X., Wang, P., et al. (2024). KARR-seq reveals cellular higher-order RNA structures and RNA-RNA interactions. Nat Biotechnol. 10.1038/s41587-023-02109-8.
34. Weng, X., Gong, J., Chen, Y., Wu, T., Wang, F., Yang, S., Yuan, Y., Luo, G., Chen, K., Hu, L., et al. (2020). Keth-seq for transcriptome-wide RNA structure mapping. Nat Chem Biol 16, 489-492. 10.1038/s41589-019-0459-3.
35. You, Q., Cheng, A. Y., Gu, X., Harada, B. T., Yu, M., Wu, T., Ren, B., Ouyang, Z., and He, C. (2021). Direct DNA crosslinking with CAP-C uncovers transcription-dependent chromatin organization at high resolution. Nat Biotechnol 39, 225-235. 10.1038/s41587-020-0643-8.
36. Dai, Q., Zhang, L. S., Sun, H. L., Pajdzik, K., Yang, L., Ye, C., Ju, C. W., Liu, S., Wang, Y., Zheng, Z., et al. (2023). Quantitative sequencing using BID-seq uncovers abundant pseudouridines in mammalian mRNA at base resolution. Nat Biotechnol 41, 344-354. 10.1038/s41587-022-01505-w.
37. Rapoport, T. A. (2007). Protein translocation across the eukaryotic endoplasmic reticulum and bacterial plasma membranes. Nature 450, 663-669. 10.1038/nature06384.
38. Zhang, X., and Shan, S. O. (2014). Fidelity of cotranslational protein targeting by the signal recognition particle. Annu Rev Biophys 43, 381-408. 10.1146/annurev-biophys-051013-022653.
39. Keenan, R. J., Freymann, D. M., Stroud, R. M., and Walter, P. (2001). The signal recognition particle. Annu Rev Biochem 70, 755-775. 10.1146/annurev.biochem.70.1.755.
40. Ast, T., Cohen, G., and Schuldiner, M. (2013). A network of cytosolic factors targets SRP-independent proteins to the endoplasmic reticulum. Cell 152, 1134-1145. 10.1016/j. cell.2013.02.003.
41. Aviram, N., Ast, T., Costa, E. A., Arakel, E. C., Chuartzman, S. G., Jan, C. H., Hassdenteufel, S., Dudek, J., Jung, M., Schorr, S., et al. (2016). The SND proteins constitute an alternative targeting route to the endoplasmic reticulum. Nature 540, 134-138. 10.1038/nature20169.
42. Satpute-Krishnan, P., Ajinkya, M., Bhat, S., Itakura, E., Hegde, R. S., and Lippincott-Schwartz, J. (2014). ER stress-induced clearance of misfolded GPI-anchored proteins via the secretory pathway. Cell 158, 522-533. 10.1016/j.cell.2014.06.026.
43. Jagannathan, S., Hsu, J. C., Reid, D. W., Chen, Q., Thompson, W. J., Moseley, A. M., and Nicchitta, C. V. (2014). Multifunctional roles for the protein translocation machinery in RNA anchoring to the endoplasmic reticulum. J Biol Chem 289, 25907-25924. 10.1074/jbc.M114.580688.
44. Wu, S., Hernandez Villegas, N. C., Sirkis, D. W., Thomas-Wright, I., Wade-Martins, R., and Schekman, R. (2023). Unconventional secretion of alpha-synuclein mediated by palmitoylated DNAJC5 oligomers. Elife 12. 10.7554/eLife.85837.
45. Sarmah, P., Shang, W., Origi, A., Licheva, M., Kraft, C., Ulbrich, M., Lichtenberg, E., Wilde, A., and Koch, H. G. (2023). mRNA targeting eliminates the need for the signal recognition particle during membrane protein insertion in bacteria. Cell Rep 42, 112140. 10.1016/j.celrep.2023.112140.
46. Costa, E. A., Subramanian, K., Nunnari, J., and Weissman, J. S. (2018). Defining the physiological role of SRP in protein-targeting efficiency and specificity. Science 359, 689-692. 10.1 126/science.aar3607.
47. Cai, Z., Cao, C., Ji, L., Ye, R., Wang, D., Xia, C., Wang, S., Du, Z., Hu, N., Yu, X., et al. (2020). RIC-seq for global in situ profiling of RNA-RNA spatial interactions. Nature 582, 432-437. 10.1038/s41586-020-2249-1.
48. Helwak, A., and Tollervey, D. (2014). Mapping the miRNA interactome by cross-linking ligation and sequencing of hybrids (CLASH). Nat Protoc 9, 711-728. 10.1038/nprot.2014.043.
49. Dudnakova, T., Dunn-Davies, H., Peters, R., and Tollervey, D. (2018). Mapping targets for small nucleolar RNAs in yeast. Wellcome Open Res 3, 120. 10.12688/wellcomeopenres.14735.2.
50. Lu, Z., Zhang, Q. C., Lee, B., Flynn, R. A., Smith, M. A., Robinson, J. T., Davidovich, C., Gooding, A. R., Goodrich, K. J., Mattick, J. S., et al. (2016). RNA Duplex Map in Living Cells Reveals Higher-Order Transcriptome Structure. Cell 165, 1267-1279. 10.1016/j.cell.2016.04.028.
51. Aw, J. G., Shen, Y., Wilm, A., Sun, M., Lim, X. N., Boon, K. L., Tapsin, S., Chan, Y. S., Tan, C. P., Sim, A. Y., et al. (2016). In Vivo Mapping of Eukaryotic RNA Interactomes Reveals Principles of Higher-Order Organization and Regulation. Mol Cell 62, 603-617. 10.1016/j.molcel.2016.04.028.
52. Engreitz, J. M., Sirokman, K., McDonel, P., Shishkin, A. A., Surka, C., Russell, P., Grossman, S. R., Chow, A. Y., Guttman, M., and Lander, E. S. (2014). RNA-RNA interactions enable specific targeting of noncoding RNAs to nascent Pre-mRNAs and chromatin sites. Cell 159, 188-199. 10.1016/j.cell.2014.08.018.
53. Soeno, Y., Taya, Y., Stasyk, T., Huber, L. A., Aoba, T., and Huttenhofer, A. (2010). Identification of novel ribonucleo-protein complexes from the brain-specific snoRNA MBII-52. RNA 16, 1293-1300. 10.1261/rna.2109710.
54. Zhang, M., Li, K., Bai, J., Van Damme, R., Zhang, W., Alba, M., Stiles, B. L., Chen, J. F., and Lu, Z. (2023). A snoRNA-tRNA modification network governs codon-biased cellular states. Proc Natl Acad Sci USA 120, e2312126120. 10.1073/pnas.2312126120.
55. Wu, T., Lyu, R., You, Q., and He, C. (2020). Kethoxal-assisted single-stranded DNA sequencing captures global transcription dynamics and enhancer activity in situ. Nat Methods 17, 515-523. 10.1038/s41592-020-0797-9.
56. Zhang, L. S., Xiong, Q. P., Pena Perez, S., Liu, C., Wei, J., Le, C., Zhang, L., Harada, B. T., Dai, Q., Feng, X., et al. (2021). ALKBH7-mediated demethylation regulates mitochondrial polycistronic RNA processing. Nat Cell Biol 23, 684-691. 10.1038/s41556-021-00709-7.
57. Zhang, L. S., Liu, C., Ma, H., Dai, Q., Sun, H. L., Luo, G., Zhang, Z., Zhang, L., Hu, L., Dong, X., and He, C. (2019). Transcriptome-wide Mapping of Internal N(7)-Methylguanosine Methylome in Mammalian mRNA. Mol Cell 74, 1304-1316 e1308. 10.1016/j.molcel.2019.03.036.
58. Cavaille, J., Nicoloso, M., and Bachellerie, J. P. (1996). Targeted ribose methylation of RNA in vivo directed by tailored antisense RNA guides. Nature 383, 732-735. 10.1038/383732a0.
59. Venema, J., and Tollervey, D. (1999). Ribosome synthesis in Saccharomyces cerevisiae. Annu Rev Genet 33, 261-311. 10.1146/annurev.genet.33.1.261.
60. Heinz, S., Benner, C., Spann, N., Bertolino, E., Lin, Y. C., Laslo, P., Cheng, J. X., Murre, C., Singh, H., and Glass, C. K. (2010). Simple combinations of lineage-determining transcription factors prime cis-regulatory elements required for macrophage and B cell identities. Mol Cell 38, 576-589. 10.1016/j.molcel.2010.05.004.
61. Mendiola, A. J. P., and LaSalle, J. M. (2021). Epigenetics in Prader-Willi Syndrome. Front Genet 12, 624581. 10.3389/fgene.2021.624581.
62. Cavaille, J. (2017). Box C/D small nucleolar RNA genes and the Prader-Willi syndrome: a complex interplay. Wiley Interdiscip Rev RNA 8. 10.1002/wrna.1417.
63. Henras, A. K., Plisson-Chastang, C., O'Donohue, M. F., Chakraborty, A., and Gleizes, P. E. (2015). An overview of pre-ribosomal RNA processing in eukaryotes. Wiley Interdiscip Rev RNA 6, 225-242. 10.1002/wrna.1269.
64. Yang, Z., Lin, J., and Ye, K. (2016). Box C/D guide RNAs recognize a maximum of 10 nt of substrates. Proc Natl Acad Sci USA 113, 10878-10883. 10.1073/pnas.1604872113.
65. Lin, J., Lai, S., Jia, R., Xu, A., Zhang, L., Lu, J., and Ye, K. (2011). Structural basis for site-specific ribose methylation by box C/D RNA protein complexes. Nature 469, 559-563. 10.1038/nature09688.
66. Rintala-Dempsey, A. C., and Kothe, U. (2017). Eukaryotic stand-alone pseudouridine synthases—RNA modifying enzymes and emerging regulators of gene expression?RNA Biol 14, 1185-1196. 10.1080/15476286.2016.1276150.
67. Martinez, N. M., Su, A., Burns, M. C., Nussbacher, J. K., Schaening, C., Sathe, S., Yeo, G. W., and Gilbert, W. V. (2022). Pseudouridine synthases modify human pre-mRNA co-transcriptionally and affect pre-mRNA processing. Mol Cell 82, 645-659 e649. 10.1016/j.molcel.2021.12.023.
68. Atzorn, V., Fragapane, P., and Kiss, T. (2004). U17/snR30 is a ubiquitous snoRNA with two conserved sequence motifs essential for 18S rRNA production. Mol Cell Biol 24, 1769-1778. 10.1128/MCB.24.4.1769-1778.2004.
69. Fayet-Lebaron, E., Atzorn, V., Henry, Y., and Kiss, T. (2009). 18S rRNA processing requires base pairings of snR30 H/ACA snoRNA to eukaryote-specific 18S sequences. EMBO J 28, 1260-1270. 10.1038/emboj.2009.79.
70. Trefts, E., Gannon, M., and Wasserman, D. H. (2017). The liver. Curr Biol 27, R1147-R1151. 10.1016/j.cub.2017.09.019.
71. Hegde, R. S., and Keenan, R. J. (2011). Tail-anchored membrane protein insertion into the endoplasmic reticulum. Nat Rev Mol Cell Biol 12, 787-798. 10.1038/nrm3226.
72. Walter, P., and Blobel, G. (1981). Translocation of proteins across the endoplasmic reticulum III. Signal recognition protein (SRP) causes signal sequence-dependent and site-specific arrest of chain elongation that is released by microsomal membranes. J Cell Biol 91, 557-561. 10.1083/jcb.91.2.557.
73. Angrisani, A., Turano, M., Paparo, L., Di Mauro, C., and Furia, M. (2011). A new human dyskerin isoform with cytoplasmic localization. Biochim Biophys Acta 1810, 1361-1368. 10.1016/j.bbagen.2011.07.012.
74. Duellman, T., Burnett, J., and Yang, J. (2015). Quantitation of secreted proteins using mCherry fusion constructs and a fluorescent microplate reader. Anal Biochem 473, 34-40. 10.1016/j.ab.2014.12.010.
75. Grasso, S., Dabene, V., Hendriks, M., Zwartjens, P., Pellaux, R., Held, M., Panke, S., van Dijl, J. M., Meyer, A., and van Rij, T. (2023). Signal Peptide Efficiency: From High-Throughput Data to Prediction and Explanation. ACS Synth Biol 12, 390-404. 10.1021/acssynbio.2c00328.
76. Flanagan, J. J., Chen, J. C., Miao, Y., Shao, Y., Lin, J., Bock, P. E., and Johnson, A. E. (2003). Signal recognition particle binds to ribosome-bound signal sequences with fluorescence-detected subnanomolar affinity that does not diminish as the nascent chain lengthens. J Biol Chem 278, 18628-18637. 10.1074/jbc.M300173200.
77. Helwak, A., Kudla, G., Dudnakova, T., and Tollervey, D. (2013). Mapping the human miRNA interactome by CLASH reveals frequent noncanonical binding. Cell 153, 654-665. 10.1016/j.cell.2013.03.043.
78. Ziv, O., Gabryelska, M. M., Lun, A. T. L., Gebert, L. F. R., Sheu-Gruttadauria, J., Meredith, L. W., Liu, Z. Y., Kwok, C. K., Qin, C. F., MacRae, I. J., et al. (2018). COMRADES determines in vivo RNA structures and interactions. Nat Methods 15, 785-788. 10.1038/s41592-018-0121-0.
79. Nguyen, T. C., Cao, X., Yu, P., Xiao, S., Lu, J., Biase, F. H., Sridhar, B., Huang, N., Zhang, K., and Zhong, S. (2016). Mapping RNA-RNA interactome and RNA structure in vivo by MARIO. Nat Commun 7, 12023. 10.1038/ncommsl2023.
80. Sharma, E., Sterne-Weiler, T., O'Hanlon, D., and Blencowe, B. J. (2016). Global Mapping of Human RNA-RNA Interactions. Mol Cell 62, 618-626. 10.1016/j.molcel.2016.04.030.
81. Sletten, A. C., Davidson, J. W., Yagabasan, B., Moores, S., Schwaiger-Haber, M., Fujiwara, H., Gale, S., Jiang, X., Sidhu, R., Gelman, S. J., et al. (2021). Loss of SNORA73 reprograms cellular metabolism and protects against steatohepatitis. Nat Commun 12, 5214. 10.1038/s41467-021-25457-y.
82. Song, J., Dong, L., Sun, H., Luo, N., Huang, Q., Li, K., Shen, X., Jiang, Z., Lv, Z., Peng, L., et al. (2023). CRISPR-free, programmable RNA pseudouridylation to suppress premature termination codons. Mol Cell 83, 139-155 e139. 10.1016/j.molcel.2022.11.011.
83. Adachi, H., Pan, Y., He, X., Chen, J. L., Klein, B., Platenburg, G., Morais, P., Boutz, P., and Yu, Y. T. (2023). Targeted pseudouridylation: An approach for suppressing nonsense mutations in disease genes. Mol Cell 83, 637-651 e639. 10.1016/j.molcel.2023.01.009.
84. Huang, C., and Yu, Y. T. (2010). Targeted 2′-0 methylation at a nucleotide within the pseudoknot of telomerase RNA reduces telomerase activity in vivo. Mol Cell Biol 30, 4368-4378. 10.1128/MCB.00384-10.
85. Hansen, H. G., Pristovsek, N., Kildegaard, H. F., and Lee, G. M. (2017). Improving the secretory capacity of Chinese hamster ovary cells by ectopic expression of effector genes: Lessons learned and future directions. Biotechnol Adv 35, 64-76. 10.1016/j.biotechadv.2016.11.008.
86. Kuo, C. C., Chiang, A. W., Shamie, I., Samoudi, M., Gutierrez, J. M., and Lewis, N. E. (2018). The emerging role of systems biology for engineering protein production in CHO cells. Curr Opin Biotechnol 51, 64-69. 10.1016/j.copbio.2017.11.015.
87. Kol, S., Ley, D., Wulff, T., Decker, M., Arnsdorf, J., Schoffelen, S., Hansen, A. H., Jensen, T. L., Gutierrez, J. M., Chiang, A. W. T., et al. (2020). Multiplex secretome engineering enhances recombinant protein production and purity. Nat Commun 11, 1908. 10.1038/s41467-020-15866-w.
88. Borth, N., Mattanovich, D., Kunert, R., and Katinger, H. (2005). Effect of increased expression of protein disulfide isomerase and heavy chain binding protein on antibody secretion in a recombinant CHO cell line. Biotechnol Prog 21, 106-111. 10.1021/bp0498241.
89. Ku, S. C., Ng, D. T., Yap, M. G., and Chao, S. H. (2008). Effects of overexpression of X-box binding protein 1 on recombinant protein production in Chinese hamster ovary and NSO myeloma cells. Biotechnol Bioeng 99, 155-164. 10.1002/bit.21562.
90. Saghaleyni, R., Malm, M., Moruzzi, N., Zrimec, J., Razavi, R., Wistbacka, N., Thorell, H., Pintar, A., Hober, A., Edfors, F., et al. (2022). Enhanced metabolism and negative regulation of ER stress support higher erythropoietin production in HEK293 cells. Cell Rep 39, 110936. 10.1016/j.celrep.2022.110936.
91. Ye, R., Zhao, H., Wang, X., and Xue, Y. (2024). Technological advancements in deciphering RNA-RNA interactions. Mol Cell. 10.1016/j.molcel.2024.06.036.
92. Ganser, L. R., Kelly, M. L., Herschlag, D., and Al-Hashimi, H. M. (2019). The roles of structural dynamics in the cellular functions of RNAs. Nat Rev Mol Cell Biol 20, 474-489. 10.1038/s41580-019-0136-0.
93. Sweeney, B. A., Hoksza, D., Nawrocki, E. P., Ribas, C. E., Madeira, F., Cannone, J. J., Gutell, R., Maddala, A., Meade, C. D., Williams, L. D., et al. (2021). R2DT is a framework for predicting and visualising RNA secondary structure using templates. Nat Commun 12, 3494. 10.1038/s41467-021-23555-5.
94. Rao, L., Xu, Y., Reineke, L. C., Bhattacharya, A., Tyryshkin, A., Shin, J. N., and Eissa, N. T. (2020). Post-Transcriptional Regulation of Alpha One Antitrypsin by a Proteasome Inhibitor. Int J Mol Sci 21. 10.3390/ijms2l 124318.
95. Untergasser, A., Cutcutache, I., Koressaar, T., Ye, J., Faircloth, B. C., Remm, M., and Rozen, S. G. (2012). Primer3--new capabilities and interfaces. Nucleic Acids Res 40, e115. 10.1093/nar/gks596.
96. Chen, S., Zhou, Y., Chen, Y., and Gu, J. (2018). fastp: an ultra-fast all-in-one FASTQ preprocessor. Bioinformatics 34, i884-i890. 10.1093/bioinformatics/bty560.
97. Martin, M. (2011). Cutadapt removes adapter sequences from high-throughput sequencing reads. EMBnet. journal 17, 10-12.
98. Dobin, A., Davis, C. A., Schlesinger, F., Drenkow, J., Zaleski, C., Jha, S., Batut, P., Chaisson, M., and Gingeras, T. R. (2013). STAR: ultrafast universal RNA-seq aligner. Bioinformatics 29, 15-21. 10.1093/bioinformatics/bts635.
99. Danecek, P., Bonfield, J. K., Liddle, J., Marshall, J., Ohan, V., Pollard, M. O., Whitwham, A., Keane, T., McCarthy, S. A., Davies, R. M., and Li, H. (2021). Twelve years of SAMtools and BCFtools. Gigascience 10. 10.1093/gigascience/giab008.
100. Quinlan, A. R., and Hall, I. M. (2010). BEDTools: a flexible suite of utilities for comparing genomic features. Bioinformatics 26, 841-842. 10.1093/bioinformatics/btq033.
101. Kim, D., Paggi, J. M., Park, C., Bennett, C., and Salzberg, S. L. (2019). Graph-based genome alignment and genotyping with HISAT2 and HISAT-genotype. Nat Biotechnol 37, 907-915. 10.1038/s41587-019-0201-4.
102. Pertea, M., Pertea, G. M., Antonescu, C. M., Chang, T. C., Mendell, J. T., and Salzberg, S. L. (2015). StringTie enables improved reconstruction of a transcriptome from RNA-seq reads. Nat Biotechnol 33, 290-295. 10.1038/nbt.3122.
103. Nassar, L. R., Barber, G. P., Benet-Pages, A., Casper, J., Clawson, H., Diekhans, M., Fischer, C., Gonzalez, J. N., Hinrichs, A. S., Lee, B. T., et al. (2023). The UCSC Genome Browser database: 2023 update. Nucleic Acids Res 51, D1188-D1195. 10.1093/nar/gkac1072.
104. Lorenz, R., Bernhart, S. H., Honer Zu Siederdissen, C., Tafer, H., Flamm, C., Stadler, P. F., and Hofacker, I. L. (2011). ViennaRNA Package 2.0. Algorithms Mol Biol 6, 26. 10.1186/1748-7188-6-26.
105. Ramirez, F., Dundar, F., Diehl, S., Gruning, B. A., and Manke, T. (2014). deepTools: a flexible platform for exploring deep-sequencing data. Nucleic Acids Res 42, W187-191. 10.1093/nar/gku365.
106. Zhang, M., Hwang, I T., Li, K., Bai, J., Chen, J. F., Weissman, T., Zou, J. Y., and Lu, Z. (2022). Classification and clustering of RNA crosslink-ligation data reveal complex structures and homodimers. Genome Res 32, 968-985. 10.1101/gr.275979.121.
107. Liao, Y., Smyth, G. K., and Shi, W. (2014). featureCounts: an efficient general purpose program for assigning sequence reads to genomic features. Bioinformatics 30, 923-930. 10.1093/bioinformatics/btt656.
108. Love, M. I., Huber, W., and Anders, S. (2014). Moderated estimation of fold change and dispersion for RNA-seq data with DESeq2. Genome Biol 15, 550. 10.1186/s13059-014-0550-8.
109. Darty, K., Denise, A., and Ponty, Y. (2009). VARNA: Interactive drawing and editing of the RNA secondary structure. Bioinformatics 25, 1974-1975. 10.1093/bioinformatics/btp250.
110. Kent, W. J., Zweig, A. S., Barber, G., Hinrichs, A. S., and Karolchik, D. (2010). BigWig and BigBed: enabling browsing of large distributed datasets. Bioinformatics 26, 2204-2207. 10.1093/bioinformatics/btq351.
111. Xu, W., Zhong, Q., Lin, D., Zuo, Y., Dai, J., Li, G., and Cao, G. (2021). CoolBox: a flexible toolkit for visual analysis of genomics data. BMC Bioinformatics 22, 489. 10.1186/s12859-021-04408-w.
112. Cui, X., Wei, Z., Zhang, L., Liu, H., Sun, L., Zhang, S. W., Huang, Y., and Meng, J. (2016). Guitar: An R/Bioconductor Package for Gene Annotation Guided Transcriptomic Analysis of RNA-Related Genomic Features. Biomed Res Int 2016, 8367534. 10.1155/2016/8367534.
113. Fang, Z., Liu, X., and Peltz, G. (2023). GSEApy: a comprehensive package for performing gene set enrichment analysis in Python. Bioinformatics 39. 10.1093/bioinformatics/btac757.
114. Chen, G., Chen, J., Liu, H., Chen, S., Zhang, Y., Li, P., Thierry-Mieg, D., Thierry-Mieg, J., Mattes, W., Ning, B., and Shi, T. (2019). Comprehensive Identification and Characterization of Human Secretome Based on Integrative Proteomic and Transcriptomic Data. Front Cell Dev Biol 7, 299. 10.3389/fcell.2019.00299.
115. Uhlen, M., Fagerberg, L., Hallstrom, B. M., Lindskog, C., Oksvold, P., Mardinoglu, A., Sivertsson, A., Kampf, C., Sjostedt, E., Asplund, A., et al. (2015). Proteomics. Tissue-based map of the human proteome. Science 347, 1260419. 10.1126/science.1260419.
116. Lomize, A. L., Lomize, M. A., Krolicki, S. R., and Pogozheva, I. D. (2017). Membranome: a database for proteome-wide analysis of single-pass membrane proteins. Nucleic Acids Res 45, D250-D255. 10.1093/nar/gkw712.
117. Kalvari, I., Nawrocki, E. P., Ontiveros-Palacios, N., Argasinska, J., Lamkiewicz, K., Marz, M., Griffiths-Jones, S., Toffano-Nioche, C., Gautheret, D., Weinberg, Z., et al. (2021). Rfam 14: expanded coverage of metagenomic, viral and microRNA families. Nucleic Acids Res 49, D192-D200. 10.1093/nar/gkaa1047.
118. Abramson, J., Adler, J., Dunger, J., Evans, R., Green, T., Pritzel, A., Ronneberger, O., Willmore, L., Ballard, A. J., Bambrick, J., et al. (2024). Accurate structure prediction of biomolecular interactions with AlphaFold 3. Nature 630, 493-500. 10.1038/s41586-024-07487-w.
119. Khatter, H., Myasnikov, A. G., Natchiar, S. K., and Klaholz, B. P. (2015). Structure of the human 80S ribosome. Nature 520, 640-645. 10.1038/naturel4427.

What is claimed is:

1. A recombinant nucleic acid encoding a protein of interest, the nucleic acid comprising a recombinant SNORA73A binding motif.

2. The recombinant nucleic acid of claim 1, wherein the SNORA73A binding motif comprises a nucleic acid sequence comprising GAGGCCCA.

3. The recombinant nucleic acid of claim 1 or 2, wherein a wildtype nucleic acid encoding the protein of interest does not comprise the SNORA73A binding motif.

4. The recombinant nucleic acid of any one of claims 1 to 3, wherein the protein of interest comprises a therapeutic protein.

5. The recombinant nucleic acid of any one of claims 1 to 4, wherein the SNORA73A binding motif is at the 3′ terminus of the recombinant nucleic acid.

6. A recombinant small nucleolar RNA (snoRNA) comprising a recombinant binding sequence complementary to a sequence on an mRNA encoding a protein of interest.

7. The recombinant snoRNA of claim 6, wherein the sequence on the mRNA is a sequence in the 3′ UTR of the mRNA.

8. A method of increasing protein secretion of a protein of interest and/or increasing membrane protein expression of a protein of interest, the method comprising contacting an mRNA encoding the protein of interest with a snoRNA comprising a recombinant binding sequence complementary to a sequence on the mRNA.

9. A method of increasing cellular protein secretion of a protein of interest and/or membrane protein expression of a protein of interest, the method comprising administering a recombinant nucleic acid encoding the protein of interest, wherein the recombinant nucleic acid comprises a recombinant SNORA73A binding motif.

10. The method of claim 9, wherein the SNORA73A binding motif comprises a nucleic acid sequence comprising GAGGCCCA.

11. The method of claim 9 or 10, wherein a wildtype nucleic acid encoding the protein of interest does not comprise the SNORA73A binding motif.

12. The method of any one of claims 9 to 11, wherein the SNORA73A binding motif is inserted at the 3′-terminus of the recombinant nucleic acid.

13. A method of decreasing cellular protein secretion of a protein of interest and/or membrane protein expression of a protein of interest, the method comprising removing a SNORA73A binding motif in a nucleic acid encoding the protein of interest.

14. The method of claim 13, wherein the SNORA73A binding motif comprises a nucleic acid sequence comprising GAGGCCCA.

15. The method of any one of claims 9 to 14, further comprising administering the nucleic acid encoding the protein of interest to a cell.

16. A method of decreasing cellular protein secretion in a population of cells, the method comprising administering a SNORA73 inhibitor to the population of cells.

17. A method of treating a disease in a patient, the method comprising administering to the patient the recombinant nucleic acid of any one of claims 1 to 7.

18. The method of claim 17, wherein the disease is characterized by a deficiency in the protein of interest.

19. The method of claim 17 or 18, wherein the disease is characterized by a secretion deficiency in the protein of interest.

20. A method of treating a disease in a patient, the method comprising administering a cell comprising the recombinant nucleic acid of any one of claims 1 to 7.


SEQUENCE LISTING (7 sequences)

SEQ ID NO: 1-2 - skipped (2 entries)

SEQ ID NO: 3 - DNA, 10 bp, synthetic construct
  source 1..10: mol_type = other DNA
gaggcccagc

SEQ ID NO: 4-5 - skipped (2 entries)

SEQ ID NO: 6 - RNA, 10 nt, synthetic construct
  source 1..10: mol_type = other RNA
gctgggcctc

SEQ ID NO: 7 - DNA, 46 bp, synthetic construct
  source 1..46: mol_type = other DNA
tcccagagtcctgtcgacaattactggggagacaaaccatgcagga